(19) United States
(12) Patent Application Publication
(10) Pub. No.: US 2005/0070558 A1
(43) Pub. Date: Mar. 31, 2005

(54) 6-Phenyldihydropyrrolopyrimidinedione derivatives

(76) Inventors: Bernat Vidal Juan, St. Cebria de Vallalta (ES); Cristina Esteve Trias, St. Pere de Riudebitlles (ES); Victor Segarra Matamoros, Barcelona (ES); Enrique Ravina Rubira, Teo (ES); Franco Fernandez Gonzalez, Santiago de Compostela (ES); Maria Isabel Loza Garcia, Santiago de Compostela (ES); Ferran Sanz Carreras, Badalona (ES)

Correspondence Address: MORRISON & FOERSTER LLP, 755 PAGE MILL RD, PALO ALTO, CA, 94304-1018, US

(21) Appl. No.: 10/481,728

(22) PCT Filed: Jun. 18, 2002
(86) PCT No.: PCT/EP02/06727

(30) Foreign Application Priority Data
Jun. 22, 2001 (ES) 200101452

(51) Int. Cl. A61K 31/519; C07D 487/02

(52) U.S. Cl. 514/265.1; 544/280

(57) ABSTRACT

6-phenylpyrrolopyrimidinedione derivatives of the formula (I), and pharmaceutically acceptable salts thereof, wherein R¹, R², R³, R⁴ and R⁵ are organic residues, L₁ is a spacer group and R⁶ is C(O) NR¹⁰R¹¹, —S(O)₂NR¹⁰R¹¹, ? [symbol] —ON═CR¹²R¹³, or a heterocyclyl, aryl? [symbol] or heteroaryl group, where R¹⁰, R¹¹, R¹² and R¹³ are organic residues, have therapeutic potential as A2 adenosine receptor inhibitors.

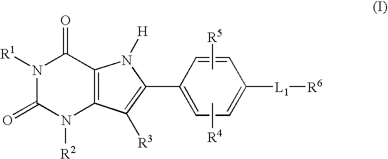

The present invention relates to antagonists of A2 adenosine receptors and in particular to antagonists of the A2b adenosine receptor subtype. Such antagonists are useful in preventing mast cell degranulation and are therefore useful in the treatment, prevention or suppression of disease states induced by activation of the A2b receptor and mast cell activation. These disease states include but are not limited to asthma, myocardial reperfusion injury, allergic reactions including but not limited to rhinitis, poison ivy induced responses, urticaria, scleroderm arthritis, other autoimmune diseases and inflammatory bowel diseases.

Adenosine regulates several physiological functions through specific cell membrane receptors. Four distinct adenosine receptors have been identified and classified as A1, A2a, A2b and A3, which are members of the G-protein coupled receptor family. The A2b adenosine receptor subtype (see review Feoktistov, I., Biaggioni, I. Pharmacol. Rev. 1997, 49, 381402) has been identified in a variety of human and murine tissues and appears to be involved in the control of vascular tone, regulation of vascular smooth muscle growth, regulation of the hepatic glucose production, modulation of intestinal tone as well as intestinal secretion and can also modulate mast cell degranulation mediating the response of human mast cells to adenosine. Adenosine A2a receptors modulate the release of GABA in the striatum which possibly regulates the activity of medium spiny neurons. Thus, A2a receptor antagonists may be a useful treatment for Parkinson's disease not only as monotherspy but also in combination with L-DOPA and dopamine agonist drugs.

It has now, surprisingly, been found that certain 6-(substituted)phenyl-1,5-dihydropyrrolo[3,2-d]pyrimidine-2,4-dione derivatives are potent and selective inhibitors of A2 adenosine receptors and in particular the A2b receptor subtype, and have efficacy in treating or preventing asthma, bronchoconstriction, allergic potentiation, inflammation or reperfusion injury, myocardial ischemia, inflammation, diarrheal diseases, brain arteriole diameter constriction, Parkinson's disease, insulin or non insulin dependent diabetes mellitus, and/or release of allergic mediators.

EP 0 480 659 relates to compounds of general formula 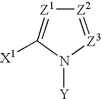
wherein each of Z¹, Z² and Z³, independently represents: a nitrogen atom, a group represented by general formula: ═C(X²)— or a group represented by general formula: ═C(X³)—. When Z² and Z³ represent a group of general formula: ═C(X²)— or a group of general formula: ═C(X³)—, X² and X³ may be combined together to form a group represented by general formula: 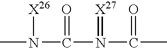
   and Y does not represent hydrogen; which possess angiotensin-IE receptor antagonizing activity for the prevention or treatment of hyperuricemia.

The present invention provides a 6-phenylpyrrolopyrimidinedione derivative of the formula (I), or a pharmaceutically acceptable salt thereof, 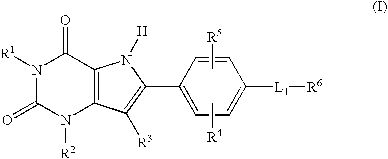
wherein:
R¹ and R² are the same or different and each represents hydrogen, a group of formula —(CH₂)ₙ—R⁷, or an alkyl group which is unsubstituted or substituted by one or more, for example 1 or 2, substituents selected from hydroxy, alkoxy, alkylthio, amino, mono- or di-alkylamino, hydroxycarbonyl, alkoxycarbonyl, acylamino, carbamoyl, alkylcarbamoyl, dihydroxyphosphoryloxy and dialkoxyphosphoryloxy groups,
wherein n is an integer of from 0 to 4 and R⁷ represents a cycloalkyl group, a phenyl group or a cyclic group which is a 3- to 7-membered, aromatic or non-aromatic ring, which contains from 1 to 4 heteroatoms selected from N, O and S and which is optionally fused to an aromatic or heteroaromatic ring, the phenyl group being unsubstituted or substituted by one or more, for example 1, 2, 3 or 4, substituents selected from halogen, alkyl, aryl, heteroaryl, heterocyclyl, hydroxy; alkylenedioxy, alkoxy, alkylthio, amino, mono- or di-alkylamino, nitro, cyano, hydroxycarbonyl, alkoxycarbonyl, acylamino, carbamoyl, alkylcarbamoyl, dihydrophosphoryloxy, dialkoxyphosphoryloxy and haloalkyl groups and the cyclic group being unsubstituted or substituted by one or more, for example 1, 2, 3 or 4, substituents selected from halogen, hydroxy, alkoxy, phenyl, alkoxycarbonyl, amino, mono-alkylamino, di-alkylamino, hydroxycarbonyl, and alkyl groups, the alkyl substituents being unsubstituted or substituted by one or more, for example 1 or 2, further substituents selected from halogen, hydroxy, alkoxy, alkylthio, acylamino, carbamoyl alkylcarbamoyl, dihydroxyphosphoryloxy, dialkoxyphosphoryloxy, hydroxyalkoxy, phenyl, alkoxycarbonyl amino, mono- and di-alkylamino and hydroxycarbonyl groups;
R³ represents hydrogen, halogen, or a nitro, alkoxycarbonyl or alkyl group, the alkyl group being unsubstituted or substituted by one or more, for example 1 or 2, substituents selected from hydroxy, halogen, alkoxy, alkylthio, amino, mono- or di-alkylamino, hydroxycarbonyl, alkoxycarbonyl, acylamino, carbamoyl and alkylcarbamoyl groups;
R⁴ and R⁵ are the same or different and each represents hydrogen, halogen, alkyl, hydroxy, alkoxy, alkylthio, dialkylaminoalkoxy, amino, mono- or dialkylamino, nitro, cyano or haloalkyl, or R⁴ and R⁵, together with the atoms to which they are attached, form a 5 to 7 membered ring containing from 0 to 4 heteroatoms selected from N, O and S;
L₁ is a direct bond or is —O—, —S—, —N(Z)-, —S(CR⁸R⁹)ₘ—, —O(CH₂)ₘ—, —O(CR⁸R⁹)ₘ—, —CH═CH—, —(CH₂)ₘ—, —(CR⁸R⁹)ₘ, —(CH₂)ₘO—, —(CR⁸R⁹)ₘO—, —(CR⁸R⁹)ₘN(Z)-, —O(CH₂)ₘO—, —O(CR⁸R⁹)ₘO—, or —N(Z)(CR⁸R⁹)ₘ— wherein m is an integer of from 1 to 6, preferably an integer of from 1 to 4, and either Z, R⁸ and R⁹ are the same or different and each represent a group selected from hydrogen, C₁-C₆ alkyl, cycloalkyl, cycloalkyl-C₁-C₆ alkyl, heterocyclyl, heterocyclyl-C₁-C₆ alkyl, aryl, aryl-C₁-C₆ alkyl, heteroaryl, heteroaryl-C₁-C₆ alkyl, hydroxy, C₁-C₆ alkoxy, halogen, cyano, C₁-C₆ alkoxycarbonyl, carbamoyl and haloalkyl, the alkyl, cycloalkyl, heterocyclyl, aryl and heteroaryl moieties being unsubstituted or substituted with one to four substituents independently selected from R¹, or Z is as defined above and R⁸ and R⁹, together with the atom to which they are attached, form a 4 to 8 membered ring; and
R⁶ represents —C(O)NR¹⁰R¹¹, —S(O)₂NR¹⁰R¹¹, —ON═CR¹²R¹³, or a heterocyclyl, aryl or heteroaryl group, the heterocyclyl, aryl and heteroaryl groups being unsubstituted or substituted with substituents R¹⁴ to R¹⁷, wherein:

R¹⁰ and R¹¹ are either
(a) the same or different, each independently representing hydrogen, an alkyl group, a cycloalkyl group or a phenyl group, wherein (i) the alkyl group is unsubstituted or substituted by one or more, for example 1 or 2, substituents selected from hydroxy, halogen, alkoxy, alkylthio, amino and mono- and di-alkylamino groups, (ii) the cycloalkyl group is optionally fused to an aromatic ring and (iii) the cycloalkyl group and the phenyl group are unsubstituted or substituted by one or more, for example 1, 2, 3 or 4, substituents selected from (1) groups of formula —(CH₂)ₙR⁷, —O—(CH₂)ₙR⁷, —S—(CH₂)ₙR⁷, —COR and —CONHR, wherein R is alkyl or —(CH₂)ₙR⁷ and n and R⁷ are as defined above, (2) groups of formula —(CH₂)ₙ—S(O)₂NR′R″ wherein n is as defined above and R′ and R″ are the same or different and are each selected from hydrogen and alkyl or form, together with the nitrogen atom to which they are attached, a 4 to 7-membered heterocyclic ring containing 1, 2 or 3 heteroatoms selected from N, O, and S, (3) groups of formula —(CH₂)ₙ—CO₂R′″ wherein n is as defined above and R′″ is hydrogen or alkyl, (4) groups of formula —N⁺R″″, wherein each R″″ is the same or different and is an alkyl group, and (5) halogen atoms and alkyl, hydroxy, alkylenedioxy, alkoxy, alkylthio, amino, mono- and di-alkylamino, nitro, cyano, hydroxycarbonyl, alkoxycarbonyl, acylamino, carbamoyl, dihydroxyphosphoryloxy, dialkoxyphosphoryloxy or haloalkyl groups, the alkyl substituents being unsubstituted or substituted by one or more, for example 1 or 2, further substituents selected from cyano, nitro, amino, hydroxy and halogen,
(b) together with the atom to which they are attached, a 3- to 7-membered ring comprising up to 4 heteroatoms selected from N, O and S, which ring is (i) optionally fused to an aromatic ring or to a heteroaromatic ring which is in turn optionally fused to an aromatic ring and is (ii) unsubstituted or substituted by one or more, for example 1, 2, 3 or 4, substituents independently selected from halogen atoms, groups of formula —X—R⁷ and —CO₂—X—R⁷ wherein X is a direct bond, a C₁-C₄ alkylene group or a carbonyl group, for example a direct bond or a C₁-C₄ alkylene group, and R⁷ is as defined above, and hydroxy, cyano, nitro, oxoalkyl, carbamoyl, hydroxycarbonyl, alkoxycarbonyl, amino, mono- and di-alkylamino; divalent alkylene and alkyl groups, the alkyl substituents being unsubstituted or substituted by one or more, for example 1 or 2, further substituents selected from hydroxy, alkoxy, hydroxyalkoxy, amino and mono- and di-alkylamino groups, and the moiety X being unsubstituted or substituted by one or two further substituents selected from phenyl, alkyl, hydroxy and thio groups and groups of formula —CO₂R′ and —CONR′R″ wherein R′ and R″ are the same or different and are hydrogen or alkyl or
(c) defined so that R¹⁰ represents hydrogen or an alkyl group and R¹¹ represents a group of formula —X—R′ wherein X and R⁷ are as defined above;
R¹² and R¹³ are defined as R¹⁰ and R¹¹ above, except that either or both of R¹² and R¹³ can be an amino, alkylamino or dialkylamino group; and
R¹⁴ to R¹⁷ are the same or different and each independently represents hydrogen, a halogen atom, a group of formula —(CH₂)ₙ—R⁷, wherein n and R⁷ are as defined above or an alkyl group, for example hydrogen, a group of formula —(CH₂)ₙ—R⁷ or an alkyl group, the alkyl group being unsubstituted or substituted by one or more, for example 1 or 2, substituents selected from hydroxy, alkoxy, alkylthio, amino, mono- or di-alkylamino, hydroxycarbonyl, alkoxycarbonyl, acylamino, carbamoyl, alkylcarbamoyl, dihydroxyphosphoryloxy, dialkoxyphosphoryloxy and haloalkyl groups, or R¹⁴ and R¹⁵ are as defined above and R¹⁶ and R¹⁷, together with the atoms to which they are attached, form a 4 to 8 membered aromatic or non-aromatic ring which contains from 0 to 4 heteroatoms selected from N, O and S, and which is unsubstituted or substituted by one or more, for example 1, 2, 3 or 4, substituents selected from halogen atoms and alkyl, hydroxy, phenyl, alkoxycarbonyl, amino, mono-alkylamino, di-alkylamino and hydroxycarbonyl groups, the alkyl substituents being unsubstituted or substituted by one or more, for example 1 or 2, further substituents selected from halogen atoms and hydroxy, alkoxy, alkylthio, acylamino, carbamoyl, alkylcarbamoyl, dihydroxyphosphoryloxy, dialkoxyphosphoryloxy, hydroxyalkoxy, phenyl, alkoxycarbonyl, amino, mono- or di-alkylamino and hydroxycarbonyl groups.

As used herein, an alkyl group or moiety is typically a linear or branched alkyl group or moiety containing from 1 to 6 carbon atoms, such as a C₁-C₄ alkyl group or moiety, for example methyl, ethyl, n-propyl, i-propyl, n-butyl, i-butyl and t-butyl. Where a group contains two or more alkyl moities, the alkyl moieties may be the same or different. When an alkyl group or moiety carries 2 or more substituents, the substituents may be the same or different.

As used herein, an alkylenedioxy group or moiety is a linear or branched group or moiety containing from 1 to 6, for example from 1 to 4, carbon atoms. Examples include methylenedioxy, ethylenedioxy, propylenedioxy and butylenedioxy. When an alkylenedioxy group or moiety carries 2 or more substituents, the substituents may be the same or different.

As used herein, an alkylene group is a divalent alkyl moiety typically having from 1 to 6, for example from 1 to 4, carbon atoms. Examples of C₁-C₄ alkylene groups include methylene, ethylene, propylene and butylene groups.

As used herein, an aryl group or moiety is typically a C₆-C₁₀ aryl group or moiety such as phenyl or naphthyl Phenyl is preferred. When an aryl group or moiety carries 2 or more substituents, the substituents may be the same or different.

As used herein, a heteroaryl group or moiety is typically a 5- to 10-membered aromatic ring, such as a 5- or 6-membered ring, containing at least one heteroatom selected from O, S and N. Examples include pyridyl pyrazinyl, pyrimidinyl pyridazinyl, furanyl, oxadiazolyl, oxazolyl, imidazolyl, thiazolyl, thiadiazolyl, thienyl, pyrazolidinyl, pyrrolyl and pyrazolyl groups. Oxadiazolyl, oxazolyl, pyridyl, pyrrolyl, imidazolyl, thiazolyl, thiadiazolyl, furanyl, pyrazinyl and pyrimidinyl groups are preferred. When a heteroaryl group or moiety carries 2 or more substituents, the substituents may be the same or different.

As used herein, a halogen is a typically chlorine, fluorine, bromine or iodine and is preferably chlorine, fluorine or bromine.

As used herein, a said alkoxy group or moiety is typically a said alkyl group attached to an oxygen atom. An alkylthio group or moiety is typically a said alkyl group attached to a thio group. A haloalkyl or haloalkoxy group is typically a said alkyl or alkoxy group substituted by one or more said halogen atoms. Typically, it is substituted by 1, 2 or 3 said halogen atoms. Preferred haloalkyl and haloalkoxy groups include perhaloalkyl and perhaloalkoxy groups such as —CX₃ and —OCX₃ wherein X is a said halogen atom. Particularly preferred haloalkyl groups are CF₃ and CCl₃. Particularly preferred haloalkoxy groups are —OCF3 and —OCCl₃.

As used herein, a cycloalkyl group typically has from 3 to 6 carbon atoms. Examples include cyclopropyl, cyclobutyl, cyclopentyl and cyclohexyl. It is preferably cyclopropyl, cyclopentyl or cyclohexyl. When a cycloalkyl group carries 2 or more substituents, the substituents may be the same or different.

As used herein, a heterocyclyl group is typically a non-aromatic, saturated or unsaturated C₅-C₁₀ carbocyclic ring in which one or more, for example 1, 2 or 3, of the carbon atoms are replaced by a heteroatom selected from N, O and S. Saturated heterocyclyl groups are preferred. Examples of suitable heterocyclyl groups include piperidinyl, piperazinyl, morpholinyl, 4,5-dihydro-oxazolyl, 3-aza-tetrahydrofuranyl, imidazolidinyl and pyrrolidinyl groups. Where a heterocyclyl group carries 2 or more substituents, the substituents may be the same or different.

As used herein, an acyl group or moiety typically has from 2 to 7 carbon atoms. Thus, it is typically a group of formula —COR wherein R is a hydrocarbyl group having from 1 to 6 carbon atoms. Preferably, it is a group of formula —COR wherein R is a said C₁-C₆ alkyl group.

Compounds of the formula (I) containing one or more chiral centre may be used in enantiomerically or diastereoisomerically pure form, or in the form of a mixture of isomers.

As used herein, a pharmaceutically acceptable salt is a salt with a pharmaceutically acceptable acid or base. Pharmaceutically acceptable acids include both inorganic acids, for example hydrochloric, sulphuric, phosphoric, diphosphoric, hydrobromic and nitric acid and organic acids, for example citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, aralkyl amines and heterocyclic amines.

Typically, at least one of R¹ and R² is hydrogen or a said alkyl group.

Preferably, R¹ and R² are the same or different and each independently represent hydrogen, a group of formula —(CH₂)ₙ—R⁷ wherein n and R⁷ are as defined above or a C₁-C₆ alkyl group which is unsubstituted or substituted by one or more, for example 1 or 2, substituents selected from hydroxy, C₁-C₆ alkoxy, C₁-C₆ alkylthio, amino and mono- and di-(C₁-C₆ alkyl)amino groups.

When R¹ or R² is a group of formula —(CH₂)ₙ—R⁷, R⁷ is preferably a C₁-C₆ cycloalkyl group or a cyclic group which is a 5- or 6-membered non-aromatic ring containing 1 or 2 heteroatoms selected from N, O and S, for example a morpholino group. In this embodiment, R⁷ is, for example, a C₃-C₆ cycloalkyl group.

More preferably, R¹ and R² are the same or different and each independently represent hydrogen, a C₁-C₄ alkyl group which is unsubsituted or substituted by 1 or 2 substituents selected from C₁-C₄ alkoxy and C₁-C₄ alkylthio substituents, a group of formula —(CH₂)ₙ—(C₃-C₆ cycloalkyl) or —(CH₂)ₙ-(morpholino) wherein n is as defined above. Examples of the more preferable R¹ and R² groups are hydrogen, a C₁-C₄ alkyl group which is unsubsituted or substituted by 1 or 2 substituents selected from C₁-C₄ alkoxy and C₁-C₄ alkylthio substituents or a group of formula —(CH₂)ₙ—(C₃-C₆ cycloalkyl) wherein n is as defined above.

More preferably still, R¹ and R² are the same or different and each independently represents a C₁-C₄ alkyl group, for example methyl, ethyl and n-propyl.

Preferably, R³ represents hydrogen, halogen or a C₁-C₆ alkyl group which is unsubstituted or substituted by one or more, for example 1 or 2, substituents selected from halogen atoms and hydroxy groups.

More preferably, R³ represents hydrogen, halogen, for example chlorine and bromine, or C₁-C₄ haloalkyl, for example —CF₃ or —CCl₃. More preferably still, R³ represents hydrogen or halogen, for example chlorine and bromine.

Typically, when R⁴ and/or R⁵ represents a haloalkyl group, the haloalkyl group is a trifluoromethyl group.

Preferably, R⁴ and R⁵ are the same or different and each represents hydrogen, halogen, C₁-C₆ alkyl, C₁-C₆ haloalkyl, hydroxy, C₁-C₆ alkoxy, C₁-C₆ alkylthio, amino or mono- or di-C₁-C₆ alkyl)amino.

More preferably, R⁴ and R⁵ are the same or different and each represents hydrogen, C₁-C₆ alkyl, hydroxy, C₁-C₆ alkoxy, C₁-C₆ alkylthio, amino or C₁-C₆ alkylamino.

More preferably still, R⁴ and R⁵ are the same or different and represent hydrogen, C₁-C₄ alkyl, C₁-C₄ alkoxy, for example methoxy, or C₁-C₄ alkylthio, for example methylthio.

Typically, when Z, R⁸ and/or R⁹ contains a cycloalkyl, heterocyclyl, aryl or heteroaryl moiety, the cycloalkyl, heterocyclyl, aryl or heteroaryl moiety is unsubstituted or substituted by 1 or 2 C₁-C₄ alkyl groups. Typically, when R⁸ and/or R⁹ contains an alkyl moiety, the alkyl moiety is unsubstituted.

When Z, R⁸ and/or R⁹ is haloalkyl, the haloalkyl group is typically —CFH₂, —CF₂H or —CF₃.

Typically, Z, R⁸ and R⁹ are the same or different and each represents hydrogen, C₁-C₄ alkyl C₃-C₆ cycloalkyl, (C₃-C₆ cycloalkyl)-(C₁-C₄ alkyl)-, phenyl or phenyl-(C₁-C₄ alkyl)-. Preferably, Z, R⁸ and R⁹ are the same or different and each represents hydrogen, C₁-C₆ alkyl, for example methyl and ethyl, or phenyl. For example; Z, R⁸ and R⁹ are the same or different and each represents C₁-C₆ alkyl, for example methyl and ethyl, or phenyl.

Preferably, L₁, is a direct bond or —O(CH₂)ₘ—, —O(CR⁸R⁹)ₘ—, —S(CR⁸R⁹)ₘ—, —CH═CH—, —(CH₂)ₘ—, —(CR⁸R⁹)ₘ—, —(CH₂)ₘO—, —(CR⁸R⁹)ₘO—, —O(CH₂)ₘO—, —(CR⁸R⁹)ₘN(Z)- or —N(Z)(CR⁸R⁹)ₘ—, for example, a direct bond or —O(CH₂)ₘ—, —O(CR⁸R⁹)ₘ—, —S(CR⁸R⁹)ₘ—, —CH═CH—, —(CH₂)ₘ—, —(CR⁸R⁹)ₘ—, —(CH₂)ₘO—, —(CR⁸R⁹)ₘO—, —(CR⁸R⁹)ₘN(Z)- or —N(Z)(CR⁸R⁹)ₘ—, wherein m is from 1 to 4, and is preferably 1, 2 or 3, R⁸ and R⁹ are as defined above and Z is hydrogen or C₁-C₄ alkyl.

More preferably, L₁, is —O(CH₂)ₘ—, —O(CR⁸R⁹)ₘ—, —CH═CH—, —(CH₂)ₘ—, —(CR⁸R⁹)ₘ—, —(CH₂)ₘO—, —C(R⁸R⁹)ₘO—, —O(CH₂)ₘO— or —(CR⁸R⁹)ₘN(Z)-, for example, —O(CH₂)ₘ—, —O(CR⁸R⁹)ₘ—, —CH═CH—, —(CH₂)ₘ—, —(CR⁸R⁹)ₘ—, —(CH₂)ₘO— or —(CR⁸R⁹)ₘO—, such as —O(CH₂)ₘ—, —O(CR⁸R⁹)ₘ—, —CH═CH—, —(CH₂)ₘ—, —(CR⁸R⁹)ₘ— or —(CH₂)ₘO—, wherein m is from 1 to 4, and is preferably 1, 2 or 3, and R³ and R⁹ are as defined above and are preferably hydrogen, C₁-C₆ alkyl, for example methyl and ethyl, or phenyl.

More preferably, L₁ is —O—CH₂—, —CH₂O— or —CH₂NH—, for example —O—CH₂.

The groups L₁ are herein written such that the left hand end of the group is attached to the phenyl moiety in formula (I) and the right hand end is attached to R⁶. Thus, for example, when L, represents —CH₂NH—, the —CH₂— moiety is attached to the phenyl ring whilst the —NH— moiety is attached to R⁶.

R¹² and R¹³ in the group R⁶ are either
(a) the same or different, each independently representing amino, alkylamino, dialkylamino, hydrogen, an alkyl group a cycloalkyl group or a phenyl group, wherein (i) the alkyl group is unsubstituted or substituted by one or more, for example 1 or 2, substituents selected from hydroxy, halogen, alkoxy, alkylthio, amino or mono- or di-alkylamino groups, (ii) the cycloalkyl group is optionally fused to an aromatic ring and (iii) the cycloalkyl group and the phenyl group are unsubstituted or substituted by one or more, for example 1, 2, 3 or 4, substituents selected from (1) groups of formula —(CH₂)ₙR⁷, —O—(CH₂)R⁷, —S—(CH₂)ₙR⁷, —COR and —CONHR, wherein R is alkyl or —(CH₂)R⁷ and n and R⁷ are as defined above, (2) groups of formula —(CH₂)ₙ—S(O)₂NR′R″ wherein n is as defined above and R′ and R″ are the same or different and are each selected from hydrogen and alkyl or form, together with the nitrogen atom to which they are attached, a 4- to 7-membered heterocyclic ring containing 1, 2 or 3 heteroatoms selected from N, O, and S, (3) groups of formula —(CH₂)ₙ—CO₂R′″, wherein n is as defined above and R″″ is hydrogen or alkyl, (4) groups of formula —N⁺R″″₃ wherein each R″″ is the same or different and is an alkyl group, and (5) halogen atoms and alkyl hydroxy, alkylenedioxy, alkoxy, alkylthio, amino, mono- or di-alkylamino, nitro, cyano, hydroxycarbonyl, alkoxycarbonyl, acylamino, carbamoyl, dihydroxyphosphoryloxy, dialkoxyphosphoryloxy or haloalkyl groups, the alkyl substituents being unsubstituted or substituted by one or more, for example 1 or 2, further substituents selected from cyano, nitro, amino, hydroxy and halogen,
(b) together with the atom to which they are attached, a 3 to 7-membered ring comprising up to 4 heteroatoms selected from N, O and S, which ring is optionally fused to one or two rings selected from aromatic and heterocyclyl rings and is unsubstituted or substituted by one or more, for example 1, 2, 3 or 4, substituents independently selected from halogen atoms, groups of formula —X—R⁷ and —CO₂—X—R⁷ wherein X is a direct bond or a C₁-C₄ alkylene group and R⁷ is as defined above, and hydroxy, cyano, nitro, oxoalkyl, carbamoyl, hydroxycarbonyl, alkoxycarbonyl, amino, mono- and di-alkylamino, divalent alkylene and alkyl groups, the alkyl substituents being unsubstituted or substituted by one or more, for example 1 or 2, further substituents selected from hydroxy, alkoxy, hydroxyalkoxy, amino or mono- or di-alkylamino groups, and the moiety X being unsubstituted or substituted by one or two further substituents selected from phenyl, alkyl hydroxy and thio groups and groups of formula —CO₂R′ and —CONR′R″ wherein R′ and R″ are the same or different and are hydrogen or alky, or
(c) defined so that R¹² represents hydrogen or an alkyl group and R¹³ represents a group of formula —X—R⁷ wherein X and R⁷ are as defined above.

Preferably, R¹² and R¹³ are the same or different and each-represents hydrogen, amino, (C₁-C₆ alkyl)amino, di-(C₁-C₆ alkyl)amino, C₁-C₆ alkyl, C₃-C₆ cycloalkyl-or phenyl, the alkyl moieties being unsubsituted or substituted by 1 or 2 subsitutents selected from hydroxy groups and halogen atoms and the cycloalkyl group and the phenyl group being unsubstituted or substituted by 1, 2, 3 or 4 substituents selected from halogen atoms and C₁-C₄ alkoxy, C₁-C₄ alkylthio, C₁-C₄ alkyl, hydroxy, C₁-C₄ haloalkyl, amino, and mono-and di-(C₁-C₄ alkyl)amino groups.

More preferably, R¹² and R¹³ are the same or different and each represents amino, mono- or di-(C₁-C₄ alkyl)amino, or phenyl, the phenyl group being unsubstituted or substituted by one or two substituents selected from halogen, for example fluorine, C₁-C₄ alkoxy, for example methoxy, C₁-C₄ alkyl, for example methyl and ethyl, hydroxy, amino, mono-(C₁-C₄ alkyl)amino and C₁-C₄ haloalkyl, for example —CF, and —CCl₁₃.

Most preferably, R¹² is amino and R¹³ is a phenyl group which is unsubstituted or substituted with a halogen atom, for example a fluorine atom.

When the moiety R⁷ is a phenyl group which carries one or more haloalkyl substituent, the or each haloalkyl substituent is typically —CF₃.

When the moiety R⁷ is a said 3- to 7-membered ring which is fused to an aromatic or heteroaromatic ring, the 3- to 7-membered ring is typically fused to an aromatic ring. Preferably, it is fused to a phenyl group. Preferably, such fused ring moieties are 5-membered heteroaromatic rings containing 1 or 2 heteroatoms selected from N, O and S, fused to a phenyl group. Examples include benzimidazole and benzothiazole.

Preferably, R⁷ is:
a C₃-C₆ cycloalkyl group;
a phenyl group which is unsubstituted or substituted with 1, 2 or 3 substituents selected from halogen, C₁-C₄ alkyl, aryl, for example phenyl, heteroaryl hydroxy, C₁-C₄ alkylenedioxy, C₁-C₄ alkoxy, C₁-C₄ alkylthio, amino, mono- and di-C₁-C₄ alkyl)amino, nitro, cyano, hydroxycarbonyl, (C₁-C₄ alkoxy)carbonyl, (C₂-C₇ acyl)amino, carbamoyl, (C₁-C₄ alkyl)carbamoyl, dihydrophosphoryloxy, di-C₁-C₄ alkoxy)phosphoryloxy and C₁-C₄ haloalkyl groups; or
a cyclic group which is a 3- to 7-membered aromatic or non-aromatic ring containing from 1 to 4, for example 1, 2 or 3, heteroatoms selected from N, O and S which is optionally fused to an aromatic ring, which group is unsubstituted or substituted by 1, 2 or 3 substituents selected from halogen, hydroxy, C₁-C₄ alkoxy, phenyl, C₁-C₄ alkoxycarbonyl, amino, mono-C₁-C₄ alkyl)amino, di-(C₁-C₄ alkyl)amino, hydroxycarbonyl and C₁-C₄ alkyl groups, the alkyl substituents being unsubstituted or substituted by 1 or 2 further substituents selected from halogen, hydroxy, C₁-C₄ alkoxy, C₁-C₄ alkylthio, C₂-C₇ acylamino, carbamoyl, C₁-C₄ alkylcarbamoyl, dihydroxyphosphoryloxy, di-C₁-C₄ alkoxy)phosphoryloxy, hydroxy-(C₁-C₄ alkoxy)-, phenyl, C₁-C₄ alkoxycarbonyl, amino, mono- and di-C₁-C₄ alkyl)amino and hydroxycarbonyl groups.

Preferably, the cyclic group is a 5- or 6-membered aromatic or non-aromatic ring containing 1 or 2 heteroatoms selected from N, O and S, which is optionally fused to a phenyl ring. More preferably, the cyclic group is a pyridinyl, pyrazinyl, pyrimidinyl, imidazolyl, thiazolyl, oxazolyl, isoxazolyl, pyrazolyl, piperidinyl, thiadiazolyl, furanyl, benzimidazolyl, benzothiazolyl, morpholino or thienyl group. For example, the cyclic group is a pyridinyl, pyrazinyl, pyrimidinyl, imaidazolyl, thiazolyl, oxazolyl, piperidinyl thiadiazolyl furanyl, benzimidazolyl or benzothiazolyl group. Further, the substituents on the cyclic group are preferably selected from halogen, for example chlorine, hydroxy, phenyl, C₁-C₄ alkoxy, amino, mono- and di-C₁-C₄ alkyl)amino, C₁-C₄ alkyl, C₁-C₄ haloalkyl, for example —CF₃, hydroxy-(C₁-C₄ alkyl)- and phenyl-C₁-C₄ alkyl)-, for example benzyl. More preferably, these subsitutents are selected from hydroxy, chlorine, C₁-C₄ alkyl, —CF₃, phenyl and benzyl.

Preferably, when R⁷ is a phenyl group, it is a phenyl group which is unsubstituted or substituted by 1 or 2 subsitutents selected from halogen, for example fluorine and chlorine, C₁-C₄ alkyl, phenyl, hydroxy, C₁-C₄ alkoxy, C₁-C₄ alkylthio, amino, mono- and di-(C₁-C₄ alkyl)amino and C₁-C₄ haloalkyl groups. More preferably, these substituents are selected from halogen, for example fluorine and chlorine, C₁-C₄ alkyl, for example methyl and ethyl, C₁-C₄ alkoxy, for example methoxy and ethoxy, hydroxy, C₁-C₄ alkylthio and —CF₃.

Typically, when the moiety X is substituted, R⁷ is a said phenyl group. More typically, when X is substituted, R⁷ is an unsubstituted phenyl group. Preferred substitutents on the moiety X include phenyl, C₁-C₄ alkyl, hydroxy, —CO₂H and —CO₂—(C₁-C₄ alkyl). More preferably, the substituents on the X moiety are selected from hydroxy, —CO₂Me, —CO₂H, methyl and phenyl.

When R¹⁰ and R¹¹ are defined according to option (a) above, R¹⁰ and/or R¹¹ can be a cycloalkyl group which is optionally fused to an aromatic ring. When the cycloalkyl group is fused to an aromatic ring, it is typically fused to a phenyl ring. Examples of such fused rings include a cyclohexyl ring fused to a phenyl ring and a cyclopentyl ring fused to a phenyl ring.

Typically, when R¹⁰ and R¹¹ are defined according to option (a) above, at least one of R¹⁰ and R¹¹ is hydrogen or C₁-C₆ alky.

When R¹⁰ and R¹¹ are defined according to option (a) above, preferably they are the same or different and each independently represent hydrogen, a C₁-C₆ alkyl group, a C₅-C₆ cycloalkyl group optionally fused to a phenyl ring or a phenyl group, the alkyl group being unsubstituted or substituted by 1 or 2 substituents selected from hydroxy, halogen, C₁-C₄ alkoxy and amino groups and the phenyl and cycloalkyl groups being unsubstituted or substituted by 1, 2, 3 or 4 substituents selected from (I) groups of formula —(CH₂)ₙR⁷, —O—(CH₂)ₙ—R⁷, —S—(CH₂)ₙ—R⁷ and —COR and —CONHR wherein R is C₁-C₆ alkyl or —(CH₂)ₙR⁷ and n and R⁷ are as defined above, (2) groups of formula —(CH₂)ₙ—S(O)₂NR′R″ wherein n is as defined above and R′ and R″ are the same or different and are each selected from hydrogen and C₁-C₆ alkyl or form, together with the N atom to which they are attached, a 4 or 5-membered saturated heterocyclic ring containing 1 or 2 heteroatoms-selected from N, O and S, (3) groups of formula —(CH₂)ₙ—CO₂R′″ wherein n is as defined above and R′″ is hydrogen or C₁-C₆ alkyl, (4) groups of formula —N⁺R″″₃ wherein each R″″ is the same or different and is a C₁-C₆ alkyl group, and (5) halogen atoms and C₁-C₆ alky, hydroxy, C₁-C₄ alkylenedioxy, C₁-C₆ alkoxy, C₁-C₆ alkythio, amino, mono- and di-(C₁-C₆ alkyl)amino, nitro, cyano, hydroxycarbonyl, (C₁-C₆ alkoxy)carbonyl, (C₂-C₇ acyl)amino, carbamoyl, and C₁-C₆ haloalkyl groups, the alkyl substituents being unsubstituted or substituted by one or more, for example 1 or 2, further substituents selected from cyano, nitro, amino, hydroxy and halogen.

More preferably, when R¹⁰ and R¹¹ are defined according to option (a) above, they are the same or different and each represent hydrogen, a C₁-C₆ alkyl group, for example methyl and ethyl, a phenyl group or a C₅-C₆ cycloalkyl group optionally fused to a phenyl ring, the alkyl group being unsubstituted or substituted by 1 or 2 substituents selected from hydroxy, halogen and amino groups and the phenyl and cycloalkyl groups being unsubstituted or substituted by 1, 2 or 3 substituents selected from (1) groups of formula —(CH₂)ₙR⁷, —O(CH₂)ₙ—R⁷, —COR and —CONHR wherein R is C₁-C₄ alkyl or —(CH₂)ₙR⁷, n is 0, 1 or 2 and R⁷ is as defined above, (2) groups of formula —(CH₂)ₙ—S(O)₂—NR′R″ wherein n is 0 or 1 and R′ and R″ are the same or different and are hydrogen or C₁-C₄ alkyl or, together with the N atom to which they are attached, form a pyrrolidinyl or piperidyl ring, (3) groups of formula —(CH₂)ₙ—CO₂R′″, wherein n is 1 or 2 and R′″ is hydrogen or C₁-C₄ alkyl, (4) groups of formula —NR″″₃ wherein each R″″ is the same or different and is a C₁-C₄ alkyl group, and (5) halogen atoms and C₁-C₄ alkyl, hydroxy, C₁-C₄ alkoxy, amino, mono- and di(C₁-C₄ alkyl)amino, nitro, cyano, hydroxycarbonyl, C₁-C₄ alkoxycarbonyl, (C₃-C₅ acyl)amino, carbamoyl and C₁-C₄ haloalkyl groups, the alkyl substituents being unsubstituted or substituted by a further substituent selected from cyano, nitro, amino, hydroxy and halogen.

Typically, when R¹⁰ and R¹¹ are as defined in the preceding paragraph, R⁷ is a phenyl group or a 5- or 6-membered aromatic or non-aromatic heterocycle having 1 or 2 heteroatoms selected from N, O and S, for example 4,5-dihydroxazolyl, the heterocycle being unsubstituted or substituted by 1 or 2 substituents selected from C₁-C₄ alkyl groups and the phenyl group being unsubstituted or substituted by 1 or 2 substituents selected from halogen atoms and C₁-C₄ alkyl and C₁-C₄ alkoxy groups.

Most preferably, when R¹⁰ and R¹¹ are defined according to option (a) above, R¹⁰ is hydrogen and R¹¹ is a phenyl group which is unsubstituted or substituted by one or two substituents selected from halogen atoms, for example fluorine and bromine, and phenyl and benzyloxy groups.

When R¹⁰ and R¹¹-are defined according to option (b) above, R¹⁰ and R¹¹ form a 3- to 7-membered heterocycle which is optionally fused to an aromatic ring or to a heteroaromatic ring which is in turn optionally fused to an aromatic ring. When the 3 to 7-membered heterocycle is fused to another ring, it is typically fused to a phenyl ring and/or to a 5- or 6-membered heterocyclic ring which is in turn optionally fused to a phenyl ring. Preferably, when the 3- to 7-membered ring is fused to another ring it is fused to a phenyl ring or to an indole group. Examples of such fused rings include 1,2,3,4-tetrahydroquinoline, 1,2,3,4-tetrahydroisoquinoline, 5,6,7,8-tetrahydro-8-aza-carbazole and 1,3,4,9-tetrahydro-beta-carbolinyl rings, for example 1,2,3,4-tetrahydroquinoline, 1,2,3,4-tetrahydroisoquinoline and 5,6,7,8-tetrahydro-8-aza-carbazole rings.

When R¹⁰ and R¹¹ are defined according to option (b) above, they typically form, together with the N atom to which they are attached, a 3- to 7-membered ring containing from 1 to 4 heteroatoms selected from N, O and S, which ring is (i) optionally fused to an aromatic ring or to a heteroaromatic ring which is in turn optionally fused to an aromatic ring and is (ii) substituted or unsubstituted by 1, 2 or 3 substituents independently selected from halogen atoms, groups of formula —X—R⁷ and —CO₂—X—R′ wherein X and R′ are as defined above, and hydroxy, cyano, nitro, carbamoyl, hydroxycarbonyl, C₁-C₆ alkoxycarbonyl, amino, mono- and di-(C₁-C₆ alkyl)amino, divalent alkylene and C₁-C₆ alkyl groups, the alkyl substituents being unsubtituted or substituted by 1 or 2 further substituents selected from hydroxy and amino groups.

More preferably, when R¹⁰ and R¹¹ are defined according to option (b) above, they form, together with the nitrogen atom to which they are attached, an aromatic or non-aromatic, for example non-aromatic, 5- or 6-membered ring containing 1 or 2 heteroatoms selected from N, O and S, which ring is optionally fused to a phenyl ring or to an indole group, and is unsubstituted or substituted by 1, 2 or 3 substituents independently selected from halogen atoms, groups of formula —X—R⁷ and —CO₂—X—R⁷ wherein X and R⁷ are as defined above, and hydroxy, cyano, nitro, C₁-C₄ alkoxycarbonyl, amino, C₁-C₂, divalent-alkylene, for example methylene and C₁-C₄ alkyl groups. The aromatic or non-aromatic ring is, for example, unsubstituted or substituted by 1, 2 or 3 substituents independently selected from halogen atoms, groups of formula —X—R⁷ and —CO₂—X—R⁷ wherein-X and R⁷ are as defined above, and hydroxy, cyano, nitro, amino, C₁-C₂ divalent alkylene, for example methylene and C₁-C₄ alkyl groups.

Typically, when R¹⁰ and R¹¹ are as defined in the preceding paragraph, the said aromatic or non-aromatic 5- or 6-membered ring is a piperidinyl, piperazinyl, pyrazolyl or morpholino ring, for example a piperidinyl, piperazinyl or morpholino ring. It can be fused to a phenyl ring to form, for example, a tetrahydroquinoline or tetrahydroisoquinoline group, or to an indole group to form, for example a 5,6,7,8-tetrahydro-8-aza-arbazole ring or a 1,3,4,9-tetrahydro-beta-carbolinyl ring. Further, when R¹⁰ and R¹¹ are as defined in the preceding paragraph, typically, X is a direct bond, a C₁-C₄ alkylene group or a carbonyl group, for example a direct bond or a C₁-C₄ alkylene group, wherein the C₁-C₄ alkylene group is unsubstituted or substituted by a phenyl group, and R⁷ is a phenyl group or a cyclic group which is a 5- or 6-membered heteroaryl group containing 1 or 2 heteroatoms selected from N, O and S, which is optionally fused to a phenyl ring, the phenyl group and the cyclic group being unsubstituted or substituted by 1 or 2 substituents selected from halogen atoms and C₁-C₄ alkyl, C₁-C₄ alkoxy and C₁-C₄ haloalkyl groups. Preferably, when R¹⁰ and R¹¹ are as defined in the preceding paragraph, X is a direct bond, —CH₂—, —CH-Ph- or a carbonyl group, for example a direct bond, —CH₂— or —CH-Ph—, and R₇ is a pyridinyl, pyrimidyl pyrazinyl, benzimidazoyl, benzothiazolyl or phenyl group, which group is unsubstituted or substituted by 1 or 2 substitutents selected from halogen atoms, and C₁-C₄ alkyl, C₁-C₄ alkoxy and —CF₃ groups.

Most preferably, when R¹⁰ and R¹¹ are defined according to option (b) above they form, together with the N atom to which they are attached, a 1,2,3,4-tetrahydroisoquinoline group, a 1,3,4,9-tetrahydro-beta-carbolinyl group, a piperidine group or a piperazine group, for example, a 1,2,3,4-tetrahydroisoquinoline group, a piperidine group or a piperazine group, the piperidine and piperazine-groups being unsubstituted or subtituted by 1 or 2 substituents selected from phenyl, pyridinyl and hydroxy groups, the phenyl and pyridinyl groups being optionally farther substituted by one or two halogen atoms, for example chlorine atoms. The piperidine and piperazine groups are, for example, substituted by one or two phenyl groups.

When R¹⁰ and R¹¹ are defined according to option (c) above, typically, R¹⁰ represents hydrogen or a C₁ to C₆ alkyl group and R¹¹ represents a group of formula —X—R⁷, wherein X and R⁷ are as defined above.

Typically, when R₁₀ and R₁₁ are defined according to option (c) above, R¹⁰ is hydrogen or a C₁-C₄ alkyl group and R¹¹ is a group of formula —X—R⁷ wherein:
—X is a direct bond, a C₁-C₄ alkylene group or a carbonyl group, for example, a direct bond or a C₁-C₄ alkylene group, wherein the C₁-C₄ alkylene group is unsubstituted or substituted by 1 or 2 substituents selected from phenyl, C₁-C₄ alkyl hydroxy, —CO₂H and —CO₂—(C₁-C₄ alkyl) groups; and
R⁷ is a C₅-C₆ cycloalkyl group, a phenyl group or a cyclic group which is a 5- or 6-membered aromatic or non-aromatic ring which contains 1 or 2 heteroatoms selected from N, O and S and which is optionally fused to a phenyl ring, the phenyl group being unsubstituted or substituted by 1 or 2 substituents selected from halogen atoms and C₁-C₄ alkyl, phenyl, hydroxy, C₁-C₄ alkoxy, C₁-C₄ alkythio, amino, mono- and di-C₁-C₄ alkyl)amino and C₁-C₄ haloalkyl groups, and the cyclic group being unsubstituted or substituted by 1 or 2 substituents selected from halogen atoms and C₁-C₄ alkyl, phenyl, phenyl-C₁-C₄-alkyl)-, hydroxy, C₁-C₄ alkoxy, C₁-C₄ alkylthio, amino, mono- and di-(C₁-C₄ alkyl)amino and C₁-C₄ haloalkyl groups,
provided that when X is substituted, R⁷ is a said unsubstituted or substituted phenyl group.

Preferably, when R¹⁰ and R¹¹ are as defined in option (c) above, R¹⁰ is hydrogen or a C₁-C₄ alkyl group and R¹⁰ is a group of formula —X—R⁷ wherein:
X is a direct bond, a C₁-C₄ alkylene group or a carbonyl group, for example, a direct bond or a C₁-C₄ alkylene group, wherein the C₁-C₄ alkylene group is unsubstituted or substituted by 1 or 2 substituents selected from C₁-C₄ alkyl hydroxy, —CO₂H and —CO₂—(C₁-C₄ alkyl) groups; and
—R⁷ is a cyclopentyl, cyclohexyl, benzimidazolyl, benzothiazolyl, thiadiazolyl, furanyl, thienyl, pyrimidinyl, pyrazinyl, isoxazolyl, pyrazolyl, pyridyl, phenyl or piperidinyl group, for example a cyclopentyl, cyclohexyl, benzimidazolyl, benzothiazolyl, thiadiazolyl, furanyl, pyridyl, phenyl or piperidinyl group, the pyridyl, pyrimidinyl piperidinyl, thiadiazolyl and furanyl groups being unsubstituted or substituted by 1 or 2 substituents selected from halogen atoms and hydroxy, C₁-C₄ alkoxy, phenyl, phenyl-C₁-C₄ alkyl- and C₁-C₄ alkyl groups, and the phenyl, benzothiazolyl and benzimidazolyl groups being unsubstituted or substituted by 1 or 2 substituents selected from halogen atoms and hydroxy, C₁-C₄ alkoxy, and C₁-C₄ alkyl groups,
provided that when X is substituted, R⁷ is an unsubstituted phenyl group.

Most preferably, when R¹⁰ and R¹¹ are as defined in option (c) above, R¹⁰ is hydrogen or a C₁-C₄ alkyl group and R¹¹ is a phenyl pyridyl, thiadiazolyl thienyl or phenylcarbonyl group, which is unsubstituted or substituted by one or two halogen atoms. In this embodiment, R¹¹ is, for example, a phenyl, pyridyl or thiadiazolyl group.

Typically, when the substituents R¹⁶ and R¹⁷ form a said 4 to 8 membered ring, R¹⁶ and R¹⁷ are either on adjacent atoms or on the same atom. When R¹⁶ and R¹⁷ are on adjacent atoms, the said 4 to 8 membered ring is typically a phenyl ring. When R¹⁶ and R¹⁷ are on the same atom, the said 4 to 8 membered ring is typically a saturated 5- or 6-membered ring, for example a cyclohexyl ring or a piperidyl ring.

Typically, R¹⁴ to R¹⁷ are the same or different and each independently represents hydrogen, a halogen atom, a group of formula —(CH₂)ₙ—R⁷ wherein n and R⁷ are as defined above, or a C₁-C₆ alkyl group, for example hydrogen, a group of formula —(CH₂)ₙ—R⁷ or a C₁-C₆ alkyl group or R¹⁴ and R¹⁵ are as defined above and R¹⁶ and R¹⁷, together with the atoms to which they are attached, form a 4 to 8 membered aromatic or non-aromatic ring which contains from 0 to 4 heteroatoms selected from N, O and S and which is unsubstituted or substituted by 1 or 2 substituents selected from halogen atoms and C₁-C₆ alkyl C₁-C₆ haloalkyl, hydroxy, phenyl, phenyl-(C₁-C₆ alkyl), amino and mono- and di-(C₁-C₆ alkyl)amino groups.

Preferably, R¹⁴ to R¹⁷ are the same or different and each independently represents hydrogen, a halogen atom, a 5- or 6-membered heteroaryl group having 1 or 2 heteroatoms selected from N, O and S, for example pyridyl, a C₁-C₄ alkyl group or a phenyl group which is unsubstituted or substituted by 1 or 2 substituents selected from halogen atoms, C₁-C₄ alkyl groups and C₁-C₄ haloalkyl groups. In this embodiment R¹⁴ to R¹⁷ are, for example, the same or different and each independently represents hydrogen, a 5- or 6-membered heteroaryl group, a C₁₋₄ alkyl group or a phenyl group, which is unsubstituted or substituted as described above. Alternatively, R¹⁴ and R¹⁵ are as defined above and R¹⁶ and R¹⁷, together with the atoms to which they are attached, form a 5- or 6-membered aromatic or non-aromatic ring which contains 0, 1 or 2 heteroatoms selected from N, O and S and which is unsubstituted or substituted by 1 or 2 substituents selected from C₁-C₄ alkyl, phenyl and phenyl-(C₁-C₄ alkyl)-substituents. More preferably, the 5- or 6-membered ring is a phenyl ring or a piperidylidene ring.

Typically, R⁶ represents —C(O)NR¹⁰R¹¹, wherein R¹⁰ and R¹¹ are as defined above, ON═CR¹²R¹³, wherein R¹² and R¹³ are as defined above, or a phenyl, heterocyclyl or heteroaryl group, for example a heterocyclyl or heteroaryl group, the phenyl, heterocyclyl and heteroaryl groups being unsubstituted or subsituted with substituents R¹⁴ to R¹⁷, as defined above.

Typically, when R⁶ is phenyl, it is unsubstituted or substituted by one halogen atom.

Typically, when R⁶ is a heterocyclyl or heteroaryl group it is a 5- or 6-membered heterocyclyl or heteroaryl group, which group contains 1, 2 or 3 heteroatoms selected from N, O and S and is unsubstituted or substituted with substituents R¹⁴ to R¹⁷, as defined above.

Preferably, the heterocyclyl or heteroaryl group is a 6-membered heteroaryl group having 1 or 2 heteroatoms selected from N, O and S, for example pyridyl, pyrimidinyl and pyrazinyl groups, or a group of formula (H) 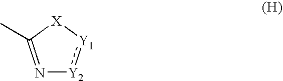
wherein X represents O, S or N, and the 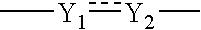
   moiety represents —N═C(R¹⁸)—, —C(R¹⁸)═N—, —C(R¹⁸)═C(R¹⁹ or —CH(R¹⁸)—CH(R¹⁹)—, wherein
—R¹⁸ and R¹⁹ are the same or different and each represents hydrogen, a group of formula —(CH₂)ₙ—R⁷ wherein n and R⁷ are as defined above, or an alkyl group, the alkyl group being unsubstituted or substituted by one or more, for example 1 or 2, substituents selected from hydroxy, alkoxy, alkylthio, amino, mono- and di-alkylamino, hydroxycarbonyl, alkoxycarbonyl, acylamino, carbamoyl, alkylcarbamoyl, dihydroxyphosphoryloxy, dialkoxyphosphoryloxy and haloalkyl groups, or R¹⁸ and R¹⁹, together with the atoms to which they are attached, form a 4 to 8 membered, aromatic or non-aromatic ring, which contains from 0 to 4 heteroatoms selected from N, O and S and which is unsubstituted or substituted by one or more, for example 1 or 2, substituents selected from halogen atoms and alkyl, hydroxy, phenyl, alkoxycarbonyl, amino, mono-alkylamino, di-alkylamino and hydroxycarbonyl groups, the alkyl substituents being unsubstituted or substituted by one or more, for example 1 or 2, further substituents selected from halogen atoms and hydroxy, alkoxy, alkylthio, acylamino, carbamoyl, alkylcarbamoyl, dihydroxyphosphoryloxy, dialkoxyphosphoryloxy, hydroxyalkoxy, phenyl, alkoxycarbonyl, amino, mono- and di-alkylamino and hydroxycarbonyl groups.

Typically, when R¹⁸ and R¹⁹ form a said 4 to 8 membered ring, R¹⁸ and R¹⁹ are either on adjacent atoms or on the same atom. When R¹⁸ and R¹⁹ are on adjacent atoms, the said 4 to 8 membered ring is typically a phenyl ring. When R¹⁸ and R¹⁹ are on the same atom, the said 4 to 8 membered ring is typically a saturated 5- or 6 membered ring, for example a cyclohexyl ring or a piperidyl ring.

Typically, R¹⁸ and R¹⁹ are the same or different and each independently represents hydrogen, a group of formula —(CH₂)—R⁷ wherein n and R⁷ are as defined above, or a C₁-C₆ alkyl group, or R¹⁸ or R¹⁹, together with the atoms to which they are attached, form a 4 to 8 membered aromatic or non-aromatic ring which contains from 0 to 4 heteroatoms selected from N, O and S and which is unsubstituted or substituted by 1 or 2 substituents selected from halogen atoms and C₁-C₆ alkyl, C₁-C₆ haloalkyl, hydroxy, phenyl, phenyl-C₁-C₆ alkyl, amino and mono- and di-C₁-C₆ alkyl)amino groups.

Preferably, R¹⁸ and R¹⁹ are the same or different and each independently represent hydrogen, a 5- or 6 membered heteroaryl group having 1 or 2 heteroatoms selected from N, O and S, for example pyridyl, a C₁-C₄ alkyl group or a phenyl group which is unsubstituted or substituted by 1 or 2 substituents selected from halogen atoms, C₁-C₄ alkyl groups and C₁-C₄ haloalkyl groups, or R¹⁸ and R¹⁹, together with the atoms to which they are attached, form a 5- or 6-membered aromatic or non aromatic ring which contains 0, 1 or 2 heteroatoms selected from N, O and S and which is unsubstituted or substituted by 1 or 2 substitutents selected from C₁-C₄ alkyl, phenyl and phenyl-(C₁-C₄ alkyl)-substituents.

Preferably, R⁶ represents —C(O)NR¹⁰R¹¹, wherein R¹⁰ and R¹¹ are as defined above, —ON═CR¹²R¹³ wherein R¹² and R¹³ are as defined above, a phenyl group which is optionally substituted by a halogen atom, or a 5- or 6& membered heteroaryl or heterocyclyl group which is optionally fused to a phenyl ring and which is unsubstituted or substituted by 1 or 2 substituents selected from phenyl, pyridyl, phenyl-(C₁-C₄alkyl)-, C₁-C₄ alkyl and piperidylidene substituents, the phenyl subsitutents being unsubstituted or substituted by 1 or 2 further substituents selected from halogen atoms and C₁-C₄ alkyl groups and the piperidylidene substituents being unsubstituted or substituted by 1 or 2 further substituents selected from phenyl, phenyl-(C₁-C₄ alkyl)- and C₁-C₄ alkyl groups.

More preferably, R⁶ represents —C(O)NR¹⁰R¹¹, a phenyl group or an oxadiazolyl group, for example a group —C(O)NR¹⁰R¹¹ or an oxadiazolyl group, wherein the oxadiazolyl group is unsubstituted or substituted by a phenyl group wherein either R¹⁰ is hydrogen and R¹¹ is a thiadiazolyl group, a pyridyl group, a phenyl group, a thienyl group or a phenylcarbonyl group, for example a thiadiazolyl group, a pyridyl group or a phenyl group, the thiadiazolyl, pyridyl, phenyl, thienyl and phenylcarbonyl groups being unsubstituted or substituted by 1 or 2 substituents selected from halogen atoms and phenyl and benzyloxy groups or R¹⁰ and R¹¹ form, together with the N atom to which they are attached, a 1, 2, 3, 4-tetrahydroisoquinoline group, a 1,3,4,9-tetrahydro-beta-carbolinyl group, a piperidine group or a piperazine group, for example a 1, 2, 3, 4-tetrahydroisoquinoline group, a piperidine group or a piperazine group, the piperidine and piperazne groups being unsubstituted or substituted by 1 or 2 substituents selected from phenyl, pyridyl and hydroxy groups, the phenyl and pyridyl groups being optionally further substituted by one or two halogen atoms, for example chlorine atoms. The piperidine and piperazine groups are, for example, substituted by one or two phenyl groups.

Preferred compounds of formula I include the compounds of formula Ia described hereinbelow, and pharmaceutically acceptable salts thereof: 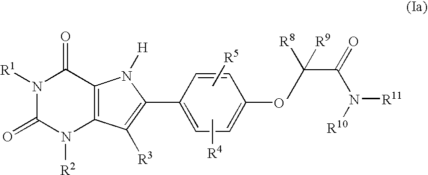
wherein R¹, R², R³, R⁴, R⁵, R⁸, R⁹, R¹⁰ and R¹¹ are as defined above.

Preferably, in the formulae (I) and (IA),
R¹ and R² are the same or different and each independently represent hydrogen, a group of formula —(CH₂)ₙ—R⁷ wherein n and R⁷ are as defined above or a C₁-C₆ alkyl group which is unsubstituted or substituted by one or more, for example 1 or 2, substituents selected from hydroxy, C₁-C₆ alkoxy, C₁-C₆ alkylthio, amino, and mono and di-C₁-C₆ alkyl)amino-groups.
R³ represents hydrogen, halogen or a C₁-C₆ alkyl group which is unsubstituted or substituted by one or more, for example 1 or 2, substituents selected from halogen atoms and hydroxy groups;
R⁴ and R⁵ are the same or different and each represent hydrogen, halogen, C₁-C₆ alkyl C₁-C₆ haloalkyl, hydroxy, C₁-C₆ alkoxy, C₁-C₆ alkylthio, amino or mono- or di-C₁—C, alkyl)amino.

Preferably, L₁ is a direct bond or —O(CH₂)ₘ—, —O(CR⁸R⁹)ₘ—, —S(CR⁸R⁹)ₘ—, —CH═CH—, —(CH₂)ₘ—, (CR⁸R⁹)ₘ—, —(CH₂)ₘO—, —(CR⁸R⁹)ₘO—, —O(CH₂)ₘ—, —(CR⁸R⁹)ₘN(Z)- or —N(Z)(CR⁸R⁹)ₘ—, for example, a direct bond or —O(CH₂)ₘ—, —O(CR⁸R⁹)ₘ—, —S(CR⁸R⁹)ₘ—, —CH═CH—, —(CH₂)ₘ—, —(CR⁵R⁹)ₘ—, —(CH₂)ₘO—, —(CR⁸R⁹)ₘO—, —(CR⁸R⁹)ₘN(Z)- or —N(Z)(CR⁸R⁹)ₘ—, wherein m is from 1 to 4, Z is hydrogen or C₁-C₄ alkyl and R¹ and R⁹ are the same or different and each represent hydrogen, C₁-C₄ alkyl, C₃-C₆ cycloalkyl, (C₃-C₆ cycloalkyl)-(C₁-C₄ alkyl)-, phenyl or phenyl-C₁-C₄ alkyl)-; and
R⁶ represents —C(O)NR¹⁰ R¹¹, —ON═CR¹²R¹³, or a phenyl, heterocyclyl or heteroaryl group, for example a heterocyclyl or heteroaryl group, the phenyl, heterocyclyl and heteroaryl groups being unsubstituted or substituted with substituents R¹⁴ to R¹⁷, wherein:
R¹⁰ and R¹¹ are either:
(a) the same or different, each independently representing hydrogen, a C₁-C₆ alkyl group, a C₅-C₆ cycloalkyl group optionally fused to a phenyl ring, or a phenyl group, the alkyl group being unsubstituted or substituted by 1 or 2 substituents selected from hydroxy, halogen, C₁-C₄ alkoxy and amino groups and the phenyl and cycloalkyl groups being unsubstituted or substituted by 1, 2, 3 or 4 substitutents selected from (1) groups of formula —(CH₂)ₙR⁷, —O—(CH₂)ₙR⁷, —S—(CH₂)ₙ—R⁷ and —COR and —CONHR wherein R is C₁-C₆ alkyl or —(CH₂)ₙR⁷ and n and R⁷ are as defined above, (2) groups of formula —(CH₂)ₙ—S(O)₂NR′R″ wherein n is as defined above and R′ and R″ are the same or different and are each selected from hydrogen and C₁-C₆ alkyl or form, together with the N atom to which they are attached, a 4- or 5-membered saturated heterocyclic ring containing 1 or 2 heteroatoms selected from N, O and S, (3) groups of formula —(CH₂)ₙ—CO₂R′″ wherein n is as defined above and R″″ is hydrogen or C₁-C₆ alkyl, (4) groups of formula —N⁺R″″, wherein each R″″ is the same or different and is a C₁-C₆ alkyl group, and (5) halogen atoms and C₁-C₆ alkyl, hydroxy, C₁-C₄ alkylenedioxy, C₁-C₆ alkoxy, C₁-C₆ alkylthio, amino, mono- and di-C₁-C₆ alkyl)amino, nitro, cyano, hydroxycarbonyl, (C₁-C₆ alkoxy)carbonyl, (C₁-C₇ acyl)amino, carbamoyl and C₁-C₆ haloalkyl groups,
(b) together with the N atom to which they are attached, a 3- to 7-membered ring containing from 1 to 4 heteroatoms selected from N, O and S which ring is (i) optionally fused to an aromatic ring or to a heteroaromatic ring which is in turn optionally fused to an aromatic ring and is (ii) substituted or unsubstituted by 1, 2 or 3 substituents independently selected from halogen atoms, groups of formula —X—R⁷ and —CO₂—X—R′ wherein X is a direct bond, a C₁-C₄ alkylene group or a carbonyl group, for example a direct bond or a C₁-C₄ alkylene group and R⁷ is as defined above, and hydroxy, cyano, nitro, carbamoyl, hydroxycarbonyl, C₁-C₆ alkoxycarbonyl, mono- and di-C₁-C₆ alkyl)amino, amino, divalent alkylene and C₁-C₆ alkyl groups, the alkyl substituents being unsubstituted or substituted by 1 or 2 further substituents selected from hydroxy and amino groups and the moiety X being unsubstituted or substituted by one or two substituents selected from phenyl, C₁-C₄ alkyl, hydroxy, —CO₂H and —CO₂—(C₁-C₄ alkyl), or
(c) defined so that R¹⁰ is hydrogen or a C₁-C₄ alkyl group and R¹¹ is a group of formula —X′—R⁷¹ wherein:
   X′ is a direct bond, a C₁-C₄ alkylene group or a carbonyl group, for example a direct bond or a C₁-C₄ alkylene group, wherein the C₁-C₄ alkylene group is unsubstituted or substituted by 1 or 2 substituents selected from phenyl, C₁-C₄ alkyl, hydroxy, —CO₂H and —CO₂—(C₁-C₄ alkyl) groups; and
   R⁷′ is a C₅-C₆ cycloalkyl group, a phenyl group or a cyclic group which is a 5- or 6-membered aromatic or non-aromatic ring which contains 1 or 2 heteroatoms selected from N, O and S and which is optionally fused to a phenyl ring, the phenyl group being unsubstituted or substituted by 1 or 2 substituents selected from halogen atoms and C₁-C₄ alkyl, phenyl, hydroxy, C₁-C₄ alkoxy, C₁-C₄ alkylthio, amino, mono- and di-(C₁-C₄ alkyl)amino and C₁-C₄ haloalkyl groups, and the cyclic group being unsubstituted or substituted by 1 or 2 substituents selected from halogen atoms and C₁-C₄ alky, phenyl, phenyl-(C₁-C₄-alkyl)-, hydroxy, C₁-C₄ alkoxy, C₁-C₄ alkylthio, amino, mono- and di-(C₁-C₄ alkyl)amino and C₁-C₄ haloalkyl groups,
provided that when X′ is substituted, R⁷′ is a said unsubstituted or substituted phenyl group,
R¹² and R¹³ are the same or different and each represent hydrogen, ammo, (C₁-C₆ alkyl)amino, di-(C₁-C₆ alkyl)amino, C₁-C₆ alkyl C₃-C₆ cycloalkyl or phenyl, the alkyl moieties being unsubstituted or substituted by 1 or 2 substitutents selected from hydroxy groups and halogen atoms and the cycloalkyl group and the phenyl group being unsubstituted or substituted by 1, 2, 3 or 4 substituents selected from halogen atoms and C₁-C₄ alkoxy, C₁-C₄ alkylthio, C₁-C₄ alkyl, hydroxy, C₁-C₄ haloalkyl, amino, and mon- and di-(C₁-C₄ alkyl)amino groups, and
R¹⁴ to R¹⁷ are the same or different and each independently represent hydrogen, a halogen atom, a group of formula —(CH₂)ₙ—R⁷ wherein n and R⁷ are as defined above, or a C₁-C₆ alkyl group, for example hydrogen, a group of formula —(CH₂)ₙ—R⁷ or a C₁-C₆ alkyl group, or R¹⁴ and R¹⁵ are as defined above and R¹⁶ and R¹⁷, together with the atoms to which they are attached, form a 4 to 8 membered aromatic or non-aromatic ring which contains from 0 to 4 heteroatoms selected from N, O and S and which is unsubstituted or substituted by 1 or 2 substituents selected from halogen atoms and C₁-C₆ alkyl, C₁-C₆ haloalkyl, hydroxy, phenyl, phenyl-C₁-C₆ alkyl)-, amino and mono- and di-C₁-C₆ alkyl)amino groups.

Particular individual compounds of the invention include:
2-[4-(2,4-Dioxo-1,3-dipropyl-2,3,4,5-tetrahydro-1H-pyrrolo[3,2-d]pyrimidin-6-yl)phenoxy]-N-phenylacetamide
6-{4-[2-Oxo-2-(4-phenylpiperazin-1-yl)ethoxy]phenyl}-1,3-dipropyl-1,5-dihydropyrrolo[3,2-d]pyrimidine-2,4-dione
2-[4-(2,4-Dioxo-1,3-dipropyl-2,3,4,5-tetrahydro-1H-pyrrolo[3,2-d]pyrimidin-6-yl)phenoxy]-N-(4-fluorophenyl)acetamide
6-{4-[2-(3,4-Dihydro-1H-isoquinolin-2-yl)₂-oxoethoxy]phenyl}-1,3-dipropyl-1,5-dihydropyrrolo[3,2-d]pyrimidine-2,4-dione
N-(4-Chlorophenyl)-2-[4-(2,4-dioxo-1,3-dipropyl-2,3,4,5-tetrahydro-1H-pyrrolo[3,2-d]pyrimidin-6-yl)phenoxy]acetamide
2-[4-(2,4-Dioxo-1,3-dipropyl-2,3,4,5-tetrahydro-1H-pyrrolo[3,2-d]pyrimidin-6-yl)phenoxy]-N-4-trifluoro methoxyphenyl)acetamide
N-(4-Cyanophenyl)-2-[4-(2,4-dioxo-1,3-dipropyl-2,3,4,5-tetrahydro-1H-pyrrolo[3,2-d]pyrimidin-6-yl)phenoxy]acetamide
4 {2-[4-(2,4-Dioxo-1,3-dipropyl-2,3,4,5-tetrahydro-1H-pyrrolo[3,2-d]pyrimidin-6-yl)phenoxy]acetylamino}benzamide
6-{4-[2-Oxo-2-(2,3,5,6-tetahydro-[1,2′]bipyrazinyl-4-yl)ethoxy]phenyl}-1,3-dipropyl-1,5 dihydropyrrolo[3,2-d]pyrimidine-2,4-dione
2-[4-(2,4-Dioxo-1,3-dipropyl-2,3,4,5-tetrahydro-1H-pyrrolo[3,2-d]pyrimidin-6-yl)phenoxy]-N-4-methoxyphenyl)acetamide
2-[4-(2,4-Dioxo-1,3-dipropyl-2,3,4,5-tetrahydro-1H-pyrrolo[3,2-d]pyrimidin-6-yl)phenoxy]-N-p-tolylacetamide
N-(4-Acetylphenyl)-2-[4-(2,4-dioxo-1,3-dipropyl-2,3,4,5-tetrahydro-1H-pyrrolo[3,2-d]pyrimidin-6-yl)phenoxy]acetamide
4-{2-[4-(2,4-Dioxo-1,3-dipropyl-2,3,4,5-tetrahydro-H-pyrrolo[3,2-d]pyrimidin-6-yl)phenoxy]acetylamino}benzoic acid ethyl ester
2-[4-(2,4-Dioxo-1,3-dipropyl-2,3,4,5-tetrahydro-1H-pyrrolo[3,2-d]pyrimidin-6-yl)phenoxy]-N-(4-trifluoromethyl phenyl)acetamide
6-(4-{2-[4-(2-Chlorophenyl)piperazin-1-yl]-2-oxo-ethoxy}phenyl)-1,3-dipropyl-1,5-dihydropyrrolo[3,2-d]pyrimidine-2,4-dione
N-(4-tert-Butylphenyl)-2-[4-(2,4-dioxo-1,3-dipropyl-2,3,4,5-tetrahydro-1H-pyrrolo[3,2-d]pyrimidin-6-yl)phenoxy]acetamide
1-{2-[4-(2,4-Dioxo-1,3-dipropyl-2,3,4,5-tetrahydro-1H-pyrrolo[3,2-d]pyrimidin-6-yl)phenoxy]acetyl}4-phenyl-piperidine-4-carbonitrile
6-{4-[2-(4-Benhydiylpiperazin-1-yl)-2-oxoethoxy]phenyl}-1,3-dipropyl-1,5-dihydropyrrolo[3,2-d]pyrimidine-2,4-dione
2-[4-(2,4-Dioxo-1,3-dipropyl-2,3,4,5-tetrahydro-1H-pyrrolo[3,2-d]pyrimidin-6-yl)phenoxy]-N-(2-hydroxy-1-phenylethyl)acetamide
N-(2-Chloro-1-phenylethyl)-2-[4-(2,4-dioxo-1,3-dipropyl-2,3,4,5-tetrahydro-1H-pyrrolo[3,2-d]pyrimidin-6-yl)phenoxy]acetamide
N-(4-Benzoylphenyl)-2-[4-(2,4-dioxo-1,3-dipropyl-2,3,4,5-tetrahydro-1H-pyrrolo[3,2-d]pyrimidin-6-yl)phenoxy]acetamide
N-(4-Cyanomethylphenyl)-2-[4-(2,4-dioxo-1,3-dipropyl-2,3,4,5-tetrahydro-1H-pyrrolo[3,2-d]pyrimidin-6-yl)phenoxy]acetamide
2-[4-(2,4-Dioxo-1,3-dipropyl-2,3,4,5-tetrahydro-1H-pyrrolo[3,2-d]pyrimidin-6-yl)phenoxy]-N-(4-sulfamoylphenyl)acetamide
2-[4-(2,4-Dioxo-1,3-dipropyl-2,3,4,5-tetrahydro-1H-pyrrolo[3,2-d]pyrimidin-6-yl)phenoxy]-N-(4-hydroxy-phenyl)acetamide
N-Biphenyl-4-yl-2-[4-(2,4-dioxo-1,3-dipropyl-2,3,4,5-tetrahydro-1H-pyrrolo[3,2-d]pyrimidin-6-yl)phenoxy]acetamide
N-(4-Benzyloxyphenyl)-2-[4-(2,4-dioxo-1,3-dipropyl-2,3,4,5-tetrahydro-1H-pyrrolo[3,2-d]pyrimidin-6-yl)phenoxy]acetamide
4-{2-[4-(2,4-Dioxo-1,3-dipropyl-2,3,4,5-tetrahydro-1H-pyrrolo[3,2-d]pyrimidin-6-yl)phenoxy]acetyl}piperazine-1-carboxylic acid benzyl ester
4-{2-[4-(2,4-Dioxo-1,3-dipropyl-2,3,4,5-tetrahydro-1H-pyrrolo[3,2-d]pyrimidin-6-yl)phenoxy]acetylamio}-N-[2-(4-methoxyphenyl)ethyl]benzamide
4-{2-[4-(2,4-Dioxo-1,3-dipropyl-2,3,4,5-tetrahydro-1H-pyrrolo[3,2-d]pyrimidin-6-yl)phenoxy]acetyl}piperazine-1-carboxylic acid phenyl ester
2-[4-(2,4-Dioxo-1,3-dipropyl-2,3,4,5-tetrahydro-1H-pyrrolo[3,2-d]pyrimidin-6-yl)phenoxy]-N-[4-pyrrolidine-1-sulfonylmethyl)phenyl]acetamide
6-{4-[2-(4,4-Diphenyl-piperidin-1-yl)-2-oxo-ethoxy]phenyl}-1,3-dipropyl-1,5-dihydropyrrolo[3,2-d]pyrimidine-2,4-dione
6-(4-{2-[4-(4-Methoxyphenyl)piperidin-1-yl]-2-oxo-ethoxy}phenyl)-1,3-dipropyl-1,5-dihydro-pyrrolo[3,2-d]pyrimidine-2,4-dione
(4-{2-[4-(2,4-Dioxo-1,3-dipropyl-2,3,4,5-tetrahydro-1H-pyrrolo[3,2-d]pyrimidin-6-yl)phenoxy]acetylamino}phenyl) acetic acid ethyl ester
6-({2-[4-(1-Methyl-1H-benzoimidazol-2-ylmethyl)piperazin-1-yl]-2-oxoethoxy}phenyl)-1,3-dipropyl-1,5-dihydropyrrolo[3,2-d]pyrimidine-2,4-dione
6-{4-[2-(3,3-Diphenylpiperazin-1-yl)-2-oxo-ethoxy]phenyl}-1,3-dipropyl-1,5-dihydro-pyrrolo[3,2-d]pyrimidine-2,4-dione
N-[4-(4,4-Dimethyl-4,5-dihydrooxazol-2-yl)phenyl]-2-[4-(2,4-dioxo-1,3-dipropyl-2,3,4,5-tetrahydro-1H-pyrrolo[3,2-d]pyrimidin-6-yl)phenoxy]acetamide
(4-{2-[4-(2,4-Dioxo-1,3-dipropyl-2,3,4,5-tetrahydro-1H-pyrrolo[3,2-d]pyrimidin-6-yl)phenoxy]acetylamino}phenyl)trimethyl ammonium
6-(4-{2-[4-(3,5-Dichloropyridin-4-yl)piperazin-1-yl]-2-oxo-ethoxy}-phenyl)-1,3-dipropyl-1,5-dihydropyrrolo[3,2-d]pyrimidine-2,4-dione
6-(4-{2-[4-(6-Chlorobenzothiazol-2-yl)piperazin-1-yl]-2-oxo-ethoxy}phenyl) 1,3-dipropyl-1,5-dihydropyrrolo[3,2-d]pyrimidine-2,4-dione
N-(4-Acetylaminophenyl)-2-[4-(2,4-dioxo-1,3-dipropyl-2,3,4,5-tetrahydro-1H-pyrrolo[3,2-d]pyrimidin-6-yl)phenoxy]acetamide
6-{4-[2-Oxo-2-(1,3,4,9-tetrahydro-β-carbolin-2-yl)ethoxy]phenyl}-1,3-dipropyl-1,5 dihydropyrrolo[3,2-d]pyrimidine-2,4-dione
2-[4-(2,4-Dioxo-1,3-dipropyl-2,3,4,5-tetrahydro-1H-pyrrolo[3,2-d]pyrimidin-6-yl)phenoxy]-N-(4-iodophenyl)acetamide
2-[4-(2,4-Dioxo-1,3-dipropyl-2,3,4,5-tetrahydro-1H-pyrrolo[3,2-d]pyrimidin-6-yl)phenoxy]-N-(2-hydroxy-2-phenylethyl)acetamide
2-[4-(2,4-Dioxo-1,3-dipropyl-2,3,4,5-tetrahydro-1H-pyrrolo[3,2-d]pyridin-6-yl)phenoxy]-N-(2-hydroxy-1-methyl-2-phenylethyl)acetamide
N-7-Cyano-3-hydroxy-2,2-dimethylchroman-4-yl)-2-[4-(2,4-dioxo-1,3-dipropyl-2,3,4,5-tetrahydro-1H-pyrrolo[3,2-d]pyrimidin-6-yl)phenoxy]acetamide
N-(1-Benzyl-3-hydroxypiperidin-3-ylmethyl)-2-[4-2,4-dioxo-1,3-dipropyl-2,3,4,5-tetrahydro-1H-pyrrolo[3,2-d]pyrimidin-6-yl)phenoxy]acetamide
2-[4-(2,4-Dioxo-1,3-dipropyl-2,3,4,5-tetrahydro-1H-pyrrolo[3,2-d]pyrimidin-6-yl)phenoxy]-N-[2-hydroxy-2-(4-hydroxyphenyl)ethyl]acetamide
2-[4-(2,4-Dioxo-1,3-dipropyl-2,3,4,5-tetrahydro-1H-pyrrolo[3,2-d]pyrimidin-6-yl)phenoxy]-N-[2-hydroxy-2-(4-hydroxy-3-hydroxymethylphenyl)ethyl]acetamide
2-[4-(2,4-Dioxo-1,3-dipropyl-2,3,4,5-tetrahydro-1H-pyrrolo[3,2-d]pyrimidin-6-yl)phenoxy]-N-2-hydroxyindan-1-yl)acetamide
6-{4-[2-Oxo-2-(6-o-tolyl-2,6-diazabicyclo[2.2.1]hept-2-yl)ethoxy]phenyl}-1,3-dipropyl-1,5-dihydropyrrolo[3,2-d]pyrimidine-2,4-dione
2-[4-(2,4-Dioxo-1,3-dipropyl-2,3,4,5-tetrahydro-1H-pyrrolo[3,2-d]pyrimidin-6-yl)phenoxy]-N-2-hydroxyphenyl)acetamide
{2-[4-(2,4-Dioxo-1,3-dipropyl-2,3,4,5-tetrahydro-H-pyrrolo[3,2-d]pyrimidin-6-yl)phenoxy]acetylamino}phenylacetic acid methyl ester
{2-[4-(2,4-Dioxo-1,3-dipropyl-2,3,4,5-tetrahydro-1H-pyrrolo[3,2-d]pyrimidin-6-yl)phenoxy]acetylamino}phenylacetic acid
(4-{2-[4-(2,4-Dioxo-1,3-dipropyl-2,3,4,5-tetrahydro-1H-pyrrolo[3,2-d]pyrimidin-6-yl)phenoxy]acetylamino}phenyl)acetic acid
N-(2-Aminoethyl)-2-[4-(1,3-dimethyl-2,4-dioxo-2,3,4,5-tetrahydro-1H-pyrrolo[3,2-d]pyrimidin-6-yl)phenoxy]acetamide
N-(4-Bromophenyl)-2-[4-(1,3-dimethyl-2,4-dioxo-2,3,4,5-tetrahydro-1H-pyrrolo[3,2-d]pyrimidin-6-yl)phenoxy]acetamide
2-[4-(1,3-Dimethyl-2,4-dioxo-2,3,4,5-tetrahydro-1H-pyrrolo[3,2-d]pyrimidin-6-yl)phenoxy]-N-phenylacetamide
2-[4-(1,3-Dimethyl-2,4-dioxo-2,3,4,5-tetrahydro-1H-pyrrolo[3,2-d]pyrimidin-6-yl)phenoxy]-N-(4-fluorophenyl)acetamide
1,3-Dimethyl-6-{4-[2-(4-methylpiperazin-1-yl)-2-oxo-ethoxy]phenyl}-1,5-dihydropyrrolo[3,2-d]pyrimidine-2,4-dione
1,3-Dimethyl-6-[4-(2-morpholin-4-yl-2-oxoethoxy)phenyl]-1,5-dihydropyrrolo[3,2-d]pyrimidine-2,4-dione
6-{4-[2-3,4-Dihydro-1H-isoquinolin-2-yl)-2-oxoethoxy]phenyl}-1,3-dimethyl-1,5-dihydropyrrolo[3,2-d]pyrimidine-2,4-dione
N-Cyclopentyl-2-[4-(1,3-dimethyl-2,4-dioxo-2,3,4,5-tetrahydro-1H-pyrrolo[3,2-d]pyrimidin-6-yl)phenoxy]acetamide
N-4-Acetylphenyl)-2-(4-1,3-dimethyl-2,4-dioxo-2,3,4,5-tetrahydro-1H-pyrrolo[3,2-d]pyrimidin-6-yl)phenoxy]acetamide
N-(1H-Benzoimidazol-2-yl)-2-[4-(1,3-dimethyl-2,4-dioxo-2,3,4,5-tetrahydro-1H-pyrrolo[3,2-d]pyrimidin-6-yl)phenoxy]acetamide
N-(4-Cyanophenyl)-2-[4-(1,3-dimethyl-2,4-dioxo-2,3,4,5-tetrahydro-1H-pyrrolo[3,2-d]pyrimidin-6-yl)phenoxy]acetamide
6-{-[2-3,4-Dihydro-2H-quinolin-1-yl)-2-oxoethoxy]phenyl}-1,3-dimethyl-1,5-dihydropyrrolo[3,2-d]pyrimidine-2,4-dione
2-[4-(1,3-Dimethyl-2,4-dioxo-2,3,4,5-tetrahydro-1H-pyrrolo[3,2-d]pyrimidin-6-yl)phenoxy]-N-[1,3,4]thiadiazol-2-ylacetamide
1,3-Dimethyl-6-{4-2-oxo-2-(4-phenylpiperazin-1-yl)ethoxy]phenyl}-1,5-dihydropyrrolo[3,2-d]pyrimidine-2,4-dione
2-[4-(1,3-Dimethyl-2,4-dioxo-2,3,4,5-tetrahydro-1H-pyrrolo[3,2-d]pyrimidin-6-yl)phenoxy]-N-(4-nitrophenyl)acetamide
6-(4-{2-[4-(4-Fluorophenyl)piperazin-1-yl]-2-oxoethoxy}phenyl)-1,3-dimethyl-1,5-dihydropyrrolo[3,2-d]pyrimidine-2,4-dione
6-{4-[2-(4-Benzylpiperazin-1-yl)₂-oxoethoxy]phenyl}-1,3 dimethyl-1,5-dihydropyrrolo[3,2-d]pyrimidine-2,4-dione
6-(4-{2-[4-(2-Methoxyphenyl)piperazin-1-yl]-2-oxoethoxy}phenyl)-1,3-dimethyl-1,5-dihydro-pyrrolo[3,2-d]pyrimidine-2,4-dione
6-(4-{2-[4-(4-Methoxyphenyl)piperazin-1-yl]-2-oxo ethoxy}phenyl)-1,3-dimethyl-1,5-dihydropyrrolo[3,2-d]pyrimidine-2,4-dione
1,3-Dimethyl-6-(4-{2-oxo-2-[4-(3-trifluoromethylphenyl)piperazin-1-yl]ethoxy}phenyl)-1,5-dihydropyrrolo[3,2-d]pyrimidine-2,4-dione
1,3-Dimethyl-6-{4-[2-oxo-2-(4-pyridin-2-yl-piperazin-1-yl)ethoxy]phenyl}-1,5-dihydropyrrolo[3,2-d]pyrimidine-2,4-dione
1,3-Dimethyl-6-{4-[2-oxo-2-(4-pyrimidin-2-yl-piperazin-1-yl)ethoxy]phenyl}-1,5 dihydropyrrolo[3,2-d]pyrimidine-2,4-dione
N-Benzyl-2-[4-(1,3-dimethyl-2,4-dioxo-2,3,4,5-tetrahydro-1H-pyrrolo[3,2-d]pyrimidin-6-yl)phenoxy]-N-methylacetamide
N-Benzyl-2-[4-(1,3-dimethyl-2,4-dioxo-2,3,4,5-tetrahydro-H-pyrrolo[3,2-d]pyrimidin-yl)phenoxy]-N-ethylacetamide
2-[4-1,3-Dimethyl-2,4-dioxo-2,3,4,5-tetrahydro-1H-pyrrolo[3,2-d]pyrimidin-6-yl)phenoxy]-N-indan-1-yl-acetamide
2-[4-1,3-Dimethyl-2,4-dioxo-2,3,4,5-tetrahydro-1H-pyrrolo[3,2-d]pyrimidin-6-yl)phenoxy]-N-(4-fluorobenzyl)acetamide
2-[4-(1,3-Dimethyl-2,4-dioxo-2,3,4,5-tetrahydro-1H-pyrrolo[3,2-d]pyrimidin-6-yl)phenoxy]-N-furan-2-ylmethyl acetamide
N-(4-Chlorobenzyl)-2-[4-(1,3-dimethyl-2,4-dioxo-2,3,4,5-tetrahydro-1H-pyrrolo[3,2-d]pyrimidin-6-yl)phenoxy]acetamide
2-[4-(1,3-Dimethyl-2,4-dioxo-2,3,4,5-tetrahydro-1H-pyrrolo[3,2-d]pyrimidin-6-yl)-phenoxy]-N-(1-phenylethyl)acetamide
2-[4-(1,3-Dimethyl-2,4-dioxo-2,3,4,5-tetrahydro-1H-pyrrolo[3,2-d]pyrimidin-6-yl)phenoxy]-N-(3-methoxybenzyl)acetamide
N-Benzyl-2-[4-(1,3-dimethyl-2,4-dioxo-2,3,4,5-tetrahydro-1H-pyrrolo[3,2-d]pyrimidin-6-yl)phenoxy]acetamide
1,3-Dimethyl-6 {4-[4-oxo-4-(6-o-tolyl-2,6-diazabicyclo[2.2.1]hept-2-yl)butoxy]phenyl}-1,5-dihydropyrrolo[3,2-d]pyrimidine-2,4-dione
2-[4-(1,3-Diethyl-2,4-dioxo-2,3,4,5-tetrahydro-1H-pyrrolo[3,2-d]pyrimidin-6-yl)phenoxy]-N-phenylacetamide
1,3-Diethyl-6-{4-[2-oxo-2-(4-phenylpiperazin-1-yl)ethoxy]phenyl}-1,5-dihydropyrrolo[3,2-d]pyrimidine-2,4-dione
N-(4-Cyanophenyl)-2-[4-(1,3-diethyl-2,4-dioxo-2,3,4,5-tetrahydro 1H-pyrrolo[3,2-d]pyrimidin-6-yl)phenoxy]acetamide
2-[4-(1-Methyl-2,4-dioxo-3-propyl-2,3,4,5-tetrahydro-1H-pyrrolo[3,2-d]pyrimidin-6-yl)phenoxy]-N-phenylacetamide
N-(4-Fluorophenyl)-2-[4-(1-methyl-2,4-dioxo-3-propyl-2,3,4,5-tetrahydro-1H-pyrrolo[3,2-d]pyrimidin-6-yl)phenoxy]acetamide
N-(4-Chlorobenzyl)-2-[4-(1-methyl-2,4-dioxo-3-propyl-2,3,4,5-tetrahydro-1H-pyrrolo[3,2-d]pyrimidin-6-yl)phenoxy]acetamide
6-{4-[2-(3,4-Dihydro-1H-isoquinolin-2-yl)-2-oxo-ethoxy]phenyl}-1-methyl-3-propyl-1,5 dihydropyrrolo[3,2-d]pyrimidine-2,4-dione
1-Methyl-6-{4-[2-oxo-2-(4-phenyl-piperazin-1-yl)ethoxy]phenyl}-3-propyl-1,5-dihydropyrrolo[3,2-d]pyrimidine-2,4-dione
6-(4-{2-[4-(4-Fuorophenyl)piperazin-1-yl]-2-oxo-ethoxy}phenyl)-1-methyl-3-propyl-1,5-dihydropyrrolo[3,2-d]pyrimidine-2,4-dione
4 {2-[4-1-Methyl-2,4-dioxo-3-propyl-2,3,4,5-tetrahydro-1H-pyrrolo[3,2-d]pyrimidin-6-yl)phenoxy]acetylamino}benzoic acid ethyl ester
6-{4-[2-(4-Hydroxy-4-phenylpiperidin-1-yl)-2-oxo ethoxy]phenyl}-1-methyl-3-propyl-1,5 dihydropyrrolo[3,2-d]pyrimidine-2,4-dione
1-{2-[4-(1-Methyl-2,4-dioxo-3-propyl-2,3,4,5-tetrahydro-1H-pyrrolo[3,2-d]pyrimidin-6-yl)phenoxy]acetyl}-4-phenylpiperidine-4-carbonitrile
N-Biphenyl-4-yl-2-[4-(1-methyl-2,4-dioxo-3-propyl-2,3,4,5-tetrahydro-1H-pyrrolo[3,2-d]pyrimidin-6-yl)phenoxy]acetamide
6-{4-[2-(4,4-Diphenylpiperidin-1-yl)-2-oxo-ethoxy]phenyl}-1-methyl-3-propyl-1,5-dihydropyrrolo[3,2-d]pyrimidine-2,4 dione
6-(4-{2-[4-(4-Methoxyphenyl)piperidin-1-yl]-2-oxo-ethoxy}phenyl)-1-methyl-3-propyl-1,5 dihydropyrrolo[3,2-d]pyrimidine-2,4-dione
(4-{2-[4-(1-Methyl-2,4-dioxo-3-propyl-2,3,4,5-tetrahydro-1H-pyrrolo[3,2-d]pyrimidin-6-yl)phenoxy]acetylamino}phenyl)acetic acid ethyl ester
6-{4-[2-(3,3-Diphenylpiperazin-1-yl)-2-oxoethoxy]phenyl}-1-methyl-3-propyl-1,5 dihydropyrrolo[3,2-d]pyrimidine-2,4-dione
6-(4-{2-[4-(6-Chlorobenzothiazol-2-yl)piperazin-1-yl]-2-oxoethoxy}phenyl)-1-methyl-3-propyl-1,5-dihydro pyrrolo[3,2-d]pyrimidine-2,4-dione
1-Methyl-6-{4-[2-oxo-2-(1,3,4,9-tetrahydro-β-carbolin-2-yl)ethoxy]phenyl}-3-propyl-1,5-dihydropyrrolo[3,2-d]pyrimidine-2,4-dione
N-(4-Iodophenyl)-2-[4-(1-methyl-2,4-dioxo-3-propyl-2,3,4,5-tetrahydro-1H-pyrrolo[3,2-d]pyrimidin-6-yl)phenoxy]acetamide
1-Methyl-6-{4-[4-oxo-4-(6-o-tolyl-2,6-diazabicyclo[2.2.1]hept-2-yl)butoxy]phenyl}-3-propyl-1,5-dihydropyrrolo[3,2-d]pyrimidine-2,4 dione
N-(4-Fluorophenyl)-2-[4-3-methyl-2,4-dioxo-1-propyl-2,3,4,5-tetrahydro-1H-pyrrolo[3,2-d]pyrimidin-6-yl)phenoxy]acetamide
2-[4-(3-Methyl-2,4-dioxo-1-propyl-2,3,4,5-tetrahydro-1H-pyrrolo[3,2-d]pyrimidin-6-yl)phenoxy]-N-phenylacetamide
N-(4-Bromophenyl)-2-[4-(3-methyl-2,4-dioxo-1-propyl-2,3,4,5-tetrahydro-1H-pyrrolo[3,2-d]pyrimidin-6-yl)phenoxy]acetamide
6-{4-[2-(3,4-Dihydro-1H-isoquinolin-2-yl)-2-oxoethoxy]phenyl}-3-methyl-1-propyl-1,5-dihydropyrrolo[3,2-d]pyrimidine-2,4-dione
N-Benzyl-2-[4-(3-methyl-2,4-dioxo-1-propyl-2,3,4,5-tetrahydro-1H-pyrrolo[3,2-d]pyrimidin-6-yl)phenoxy]acetamide
N-Benzyl-N-methyl-2-[4-(3-methyl-2,4-dioxo-1-propyl-2,3,4,5-tetrahydro-1H-pyrrolo[3,2-d]pyrimidin-6-yl)phenoxy]acetamide
3-Methyl-6-{4-[2-oxo-2-(4-phenylpiperazin-1-yl)-ethoxy]phenyl}-1-propyl-1,5-dihydropyrrolo[3,2-d]pyrimidine-2,4-dione
6-{4-[2-(4-Benzylpiperazin-1-yl)-2-oxoethoxy]phenyl}-3-methyl-1-propyl-1,5-dihydropyrrolo[3,2-d]pyrimidine-2,4-dione
3-Methyl-6-{4-[4-oxo-4-(6-o-tolyl-2,6-diazabicyclo[2.2.1]hept-2-yl)butoxy]phenyl}-1-propyl-1,5-dihydropyrrolo[3,2-d]pyrimidine-2,4-dione
N-Cyclopentyl-2-{4-[1-3-methoxypropyl)-3-methyl-2,4-dioxo-2,3,4,5-tetrahydro-1H-pyrrolo[3,2-d]pyrimidin-6-yl]phenoxy}acetamide
2-{4-[1-3-Methoxypropyl)-3-methyl-2,4-dioxo-2,3,4,5-tetrahydro-1H-pyrrolo[3,2-d]pyrimidin-6-yl]phenoxy}-N-phenylacetamide
2-[4-(3-Isobutyl-1-methyl-2,4-dioxo-2,3,4,5-tetrahydro-1H-pyrrolo[3,2-d]pyrimidin-6-yl)phenoxy]-N-phenylacetamide
3-Isobutyl-1-methyl-6-{4-[2-oxo-2-4-phenylpiperazin-1-yl)ethoxy]phenyl}-1,5-dihydropyrrolo[3,2-d[pyrimidine-2,4-dione
4-{2-[4-(2,4-Dioxo-1-propyl-2,3,4,5-tetrahydro-1H-pyrrolo[3,2-d]pyrimidin-6-yl)phenoxy]acetylamino}benzoic acid ethyl ester
6-(4-{2-[4-(4-Methoxyphenyl)piperidin-1-yl]-2-oxo-ethoxy}phenyl)-1-propyl-1,5-dihydropyrrolo[3,2-d]pyrimidine-2,4-dione
6-(4-{2-[4-(4-(Methoxyphenyl)piperazin-1-yl]-2-oxo-ethoxy}phenyl)-1-propyl-1,5-dihydropyrrolo[3,2-d]pyrimidine-2,4-dione
N-4-Bromophenyl)-2-[4-(2,4-dioxo-1-propyl-2,3,4,5-tetrahydro-1H-pyrrolo[3,2-d]pyrimidin-6-yl)phenoxy]acetamide
2-[4-(2,4-Dioxo-1-propyl-2,3,4,5-tetrahydro-1H-pyrrolo[3,2-d]pyrimidin-6-yl)phenoxy]-N-(4-fluorophenyl)acetamide
2-{4-[1,3-Bis(2-methoxyethyl)-2,4-dioxo-2,3,4,5-tetrahydro-1H-pyrrolo[3,2-d]pyrimidin-6-yl]phenoxy}-N-phenylacetamide
2-{4-[1,3-Bis(2-methoxyethyl)-2,4-dioxo-2,3,4,5-tetrahydro-1H-pyrrolo[3,2-d]pyrimidin-6-yl]phenoxy}-N-(4-fluorophenyl)acetamide
2-{4-[1,3-Bis(2-methoxyethyl)-2,4-oxo-2,3,4,5-tetrahydro-1H-pyrrolo[3,2-d]pyrimidin-6-yl]phenoxy}-N-(4-bromophenyl)acetamide
1,3-Bis(2-methoxyethyl)-6-{4-[2-oxo-2-(4-phenylpiperazin-1-yl)ethoxy]phenyl}-1,5-dihydropyrrolo[3,2-d]pyrimidine-2,4-dione
6-{4-[2-(3,4-Dihydro-1H-isoquiolin-2-yl)₂-oxoethoxy]phenyl}-1,3-bis(2-methoxyethyl)-1,5-dihydropyrrolo[3,2-d]pyrimidine-2,4-dione
2-[4 (1,3-Bis(cyclopropylmethyl)-2,4-dioxo-2,3,4,5-tetrahydro-1H-pyrrolo[3,2-d]pyrimidin-6-yl)phenoxy]-N-phenylacetamide
2-[4-(1,3-Bis(cyclopropylmethyl)-2,4-dioxo-2,3,4,5-tetrahydro-1H-pyrrolo[3,2-d]pyrimidin-6-yl)phenoxy]-N-(4-fluorophenyl)acetamide
2-[4-(1,3-Bis(cyclopropylmethyl)-2,4-dioxo-2,3,4,5-tetrahydro-1H-pyrrolo[3,2-d]pyrimidin-6-yl)phenoxy]-N-(4-bromophenyl)acetamide
1,3-Bis(cyclopropylmethyl)-6-{-[2-oxo-2-(4-phenylpiperazin-1-l)ethoxy]phenyl}-1,5-dihydropyrrolo[3,2-4 pyrimidine-2,4-dione
1,3-Bis(cyclopropylmethyl)-6 {4-[2-(3,4-dihydro-1H-isoquinolin-2-yl)-2-oxoethoxy]phenyl}-1,5-dihydropyrrolo[3,2-d]pyrimidine-2,4-dione
2-[4-(7-Chloro-1,3-dimethyl-2,4-dioxo-2,3,4,5-tetrahydro-1H-pyrrolo[3,2-d]pyrimidin-6-yl)phenoxy]-N-(4-cyanophenyl)acetamide
2-[4-(7-Bromo-2,4-dioxo-1,3-propyl-2,3,4,5-tetrahydro-1H-pyrrolo[3,2-d]pyrimidin-6-yl)phenoxy]-N-phenylacetamide
2-[4-(7-Bromo-2,4-dioxo-1,3-dipropyl-2,3,4,5-tetrahydro-1H-pyrrolo[3,2-d]pyrimidin-6-yl)phenoxy]-N-(4-fluorophenyl)acetamide
2-[4-(7-Chloro-2,4-dioxo-1,3 propyl-2,3,4,5-tetrahydro-1H-pyrrolo[3,2-d]pyrimidin-6-yl)phenoxy]-N-4-fluorophenyl)acetamide
2-[4-(7-Chloro-2,4-dioxo-1,3-dipropyl-2,3,4,5-tetrahydro-1H-pyrrolo[3,2-d]pyrimidin-6-yl)phenoxy]-N-phenylacetamide
N-(4-Bromophenyl)-2-[4-(7-chloro-2,4-dioxo-1,3-dipropyl-2,3,4,5-tetrahydro-1H-pyrrolo[3,2-d]pyrimidin-6-yl)phenoxy]acetamide
2-[4-(7-Chloro-2,4-dioxo-1,3-dipropyl-2,3,4,5-tetrahydro-1H-pyrrolo[3,2-d]pyrimidin-6-yl)phenoxy]-N-(2-chlorophenyl)acetamide
2-[4-(7-Chloro-2,4-dioxo-1,3-dipropyl-2,3,4,5-tetrahydro-1H-pyrrolo[3,2-d]pyrimidin-6-yl)phenoxy]-N-(4-chlorophenyl)acetamide
2-[4-(7 Chloro-2,4-dioxo-1,3-dipropyl-2,3,4,5-tetrahydro-1H-pyrrolo[332-d]pyrimidin-6-yl)phenoxy]-N-(2-fluorophenyl)acetamide
2-[4-(7-Chloro-2,4-dioxo 1,3-dipropyl-2,3,4,5-tetrahydro-H-pyrrolo[312-d]pyrimidin-6-yl)phenoxy]-N-(4-fluorobenzyl)acetamide
2-[4-(7-Chloro-2,4-dioxo-1,3-dipropyl-2,3,4,5-tetrahydro-1H-pyrrolo[3,2-d]pyrimidin-6-yl)phenoxy]-N-(4-methoxyphenyl)acetamide
N-Benzyl-2-[4-(7-chloro-2,4 dioxo-1,3-dipropyl-2,3,4,5-tetrahydro-H-pyrrolo[3,2-d]pyrimidin-6-yl)phenoxy]acetamide
2-[4-(7-Chloro-2,4-dioxo-1,3-dipropyl-2,3,4,5-tetrahydro-1H-pyrrolo[3,2-d]pyrimidin-6-yl)phenoxy]-N-p-tolylacetamide
2-[4-(7-Chloro-2, oxo-1,3-dipropyl-2,3,4,5-tetrahydro-1H-pyrrolo[3,2-d]pyrimidin-6-yl)phenoxy]-N-3-fluorophenyl)acetamide
2-[4-(1,3-Dimethyl-2,4 oxo-2,3,4,5-tetrahydro-1H-pyrrolo[3,2-d]pyrimidin-6-yl)-3-methoxyphenoxy]-N-phenyl-acetamide
2-[4,1,3-Dimethyl-2,4-dioxo-2,3,4,5-tetrahydro-1H-pyrrolo[3,2-d]pyrimidin-6-yl)-3-methoxy-phenoxy]-N-(3-fluorophenyl)acetamide
N-(4-Chlorobenzyl)-2-(4-(1,3-dimethyl-2,4-dioxo-2,3,4,5-tetrahydro-H-pyrrolo[3,2-d]pyrimidin-6-yl)-3-methoxyphenoxy]acetamide
6-{4-[2-(3,4-Dihydro-1H-isoquinolin-2-yl)-2-oxo-ethoxy]-2-methoxyphenyl}-1,3-dimethyl-1,5-dihydropyrrolo[3,2-d]pyrimidine-2,4-dione
6-{2-Methoxy-4-[2-oxo-2-(4-phenylpiperazin-1-yl)ethoxy]phenyl}-1,3-dimethyl-1,5-dihydropyrrolo[3,2-d]pyrimidine-2,4-dione
N-(4-Cyanophenyl)-2-[4-(1,3-dimethyl-2,4-dioxo-2,3,4,5-tetrahydro-1H-pyrrolo[3,2-d]pyrimidin-6-yl)-3-methoxyphenoxy]acetamide
N-(4-Bromophenyl)-2-[4-(1,3-dimethyl-2,4-dioxo-2,3,4,5-tetrahydro-1H-pyrrolo[3,2-d]pyrimidin-6-yl)-3-methoxyphenoxy]acetamide
6-(2-Methoxy-4-{2-[4-(4-methoxyphenyl)piperidin-1-yl]-2-oxoethoxy}phenyl)-1,3-dimethyl-1,5-dihydropyrrolo[3,2-d]pyrimidine-2,4-dione
6-(2-Methoxy-4-{2-[4-(4-methoxyphenyl)-piperazin-1-yl]-2-oxoethoxy}phenyl)-1,3-dimethyl-1,5 dihydropyrrolo[3,2-d]pyrimidine-2,4-dione
2-[4-1,3-Dimethyl-2,4 dioxo-2,3,4,5-tetrahydro-1H-pyrrolo[3,2-pyrimidin-6-yl)-2-methoxyphenoxy]-N-phenyl acetamide
2-[4-(1,3-Dimethyl-2,4-dioxo-2,3,4,5-tetrahydro-1H-pyrrolo[3,2-d]pyrimidin-6-yl)-2-methoxyphenoxy]-N-(4-fluorophenyl)acetamide
N-(4-Chlorobenzyl)-2-[4-(1,3-dimethyl-2,4-dioxo-2,3,4,5-tetrahydro-1H-pyrrolo[3,2-d]pyrimidin-6-yl)-2-methoxyphenoxy-acetamide
6-{4-[2-3,4-Dihydro-1H-isoquinolin-2-yl)-2-oxoethoxy]-3-methoxyphenyl}-1,3-dimethyl-1,5-dihydropyrrolo[3,2-d]pyrimidine-2,4-dione
6-{3-Methoxy 4-[2-oxo-2-(4-phenylpiperazine-1-yl)ethoxy]phenyl}-1,3-dimethyl-1,5-dihydropyrrolo[3,2-d-]pyrimidine-2,4-dione
N-(4-Cyanophenyl)-2-[4 (1,3-dimethyl-2,4-dioxo-2,3,4,5-tetrahydro-1H-pyrrolo[3,2-d]pyrimidin-6-yl)₂-methoxyphenoxy]acetamide
N-(4-Bromophenyl)-2-[4-(1,3-dimethyl-2,4-dioxo-2,3,4,5-tetrahydro-1H-pyrrolo[3,2-pyrimidin-6-yl)-2-methoxyphenoxy]acetamide
4-{2-[4-(1,3-Dimethyl-2,4-dioxo-2,3,4,5-tetrahydro-1H-pyrrolo[3,2-d]pyrimidin-6-yl)-2-methoxyphenoxy]acetylamino}benzoic acid ethyl ester
6-(3-Methoxy-4-{2-[4-(4-methoxyphenyl)piperidin-1-yl]-2-oxoethoxy}phenyl)-1,3-dimethyl-1,5-dihydropyrrolo[3,2-d]pyrimidine-2,4-dione
6-(3-Methoxy-4-{2-[4-(4-methoxyphenyl)piperazin-1-yl]-2-oxoethoxy}phenyl)-1,3 dimethyl-1,5-dihydropyrrolo[3,2-d]pyrimidine-2,4-done
2-[4-(2,4-Dioxo-1,3-dipropyl-2,3,4,5-tetrahydro-1H-pyrrolo[3,2-d]pyrimidin-6-yl)phenoxy]-N-phenylpropionamide
6-{4-[2-(3,4-Dihydro-1H-isoquinolin-2-yl)-1-methyl-2-oxoethoxy]phenyl}-1,3-dipropyl-1,5-dihydropyrrolo[3,2-d]pyrimidine-2,4-dione
6-{4-[1-Methyl-2-oxo-2-(4-phenylpiperazin-1-yl)ethoxy]phenyl}-1,3-dipropyl-1,5-dihydropyrrolo[3,2-d]pyrimidine-2,4-dione
N-(4-Chlorobenzyl)-2-[4-(2,4-dioxo-1,3-dipropyl-2,3,4,5-tetrahydro-1H-pyrrolo[3,2-d]pyrimidin-6-yl)phenoxy]propionamide
2-[4-(2,4-Dioxo-1,3-dipropyl-2,3,4,5-tetrahydro-1H-pyrrolo[3,2-d]pyrimidin-6-yl)phenoxy]-N-(4-fluorophenyl)propionamide
2-[4-(2,4-Dioxo-1,3-dipropyl-2,3,4,5-tetrahydro-1H-pyrrolo[3,2-d]pyrimidin-6-yl)phenoxy]-N-(4-methoxyphenyl)propionamide
2-[4-(1,3-Dimethyl-2,4-dioxo-2,3,4,5-tetrahydro-1H-pyrrolo[3,2-d]pyrimidin-6-yl)phenoxy]-N-phenylpropionamide
2-[4-(1,3-Dimethyl-2,4-dioxo-2,3,4,5-tetrahydro-1H-pyrrolo[3,2-d]pyrimidin-6-yl)phenoxy]-N-(4-fluorophenyl)propionamide
N-4-Bromophenyl)-2-[4-(1,3-dimethyl-2,4-dioxo-2,3,4,5-tetrahydro-1H-pyrrolo[3,2-d]pyrimidin-6-yl)phenoxy]propionamide
1,3-Dimethyl-6-{4-[1-methyl-2-oxo-2-(4-phenyl piperazin-1-yl)ethoxy]phenyl}-1,5-dihydropyrrolo[3,2-d]pyrimidine-2,4-dione
6 {4-[2-3,4-Dihydro-1H-isoquinolin-2-yl)-1-methyl-2-oxoethoxy]phenyl}-1,3-dimethyl-1,5-dihydropyrrolo[3,2-d]pyrimidine-2,4-dione
2-[4-(2,4-Dioxo-1,3-dipropyl-2,3,4,5-tetrahydro-1H-pyrrolo[3,2-d]pyrimidin-6-yl)phenoxy]-N-phenylbutyramide
2-[4-(2,4-Dioxo-1,3-dipropyl-2,3,4,5-tetrahydro-1H-pyrrolo[3,2-d]pyrimidin-6-yl)phenoxy]-[(4-fluorophenyl)butyramide
N-(4-Bromophenyl)-2-[4-(2,4-dioxo-1,3-propyl-2,3,4,5-tetrahydro-1H-pyrrolo[3,2-d]pyrimidin-6-yl)phenoxy]butyramide
6-{4-[1-(4-Phenylpiperazine-1-carbonyl)propoxy]phenyl}-1,3-dipropyl-1,5-dihydropyrrolo[3,2-d]pyrimidine-2,4-dione
6-{4-[1-(3,4-Dihydro-1H-isoquinoline-2-carbonyl)propoxy]phenyl}-1,3-dipropyl-1,5-dihydropyrrolo[3,2-d]pyrimidine-2,4-dione
2-[4-(2,4-Dioxo-1,3-dipropyl-2,3,4,5-tetrahydro-1H-pyrrolo[3,2-d]pyrimidin-6-yl)phenoxy]-2-methyl-N-phenyl propionamide
2-[4-(2,4-Dioxo-1,3-dipropyl-2,3,4,5-tetrahydro-1H-pyrrolo[3,2-d]pyrimidin-6-yl)phenoxy]-N-(4-fluorophenyl)-2-methylpropionamide
N-(4-Bromophenyl)-2-[4-(2,4-dioxo-1,3-dipropyl-2,3,4,5-tetrahydro-1H-pyrrolo[3,2-d]pyrimidin-6-yl)phenoxy]-2-methylpropionamide
6-{4-[1,1-Dimethyl-2-oxo-2-4-phenylpiperazin-1-yl)ethoxy]phenyl}-1,3-dipropyl-1,5-dihydropyrrolo[3,2-4 pyrimidine-2,4-dione
6-{4-[2-(3,4-Dihydro-1H-isoquinolin-2-yl)-1,1-dimethyl-2-oxoethoxy]phenyl}-1,3-dipropyl-1,5-dihydropyrolo[3,2-d]pyrimidine-2,4 dione
2-[4-(2,4-Dioxo-1,3 dipropyl-2,3,4,5-tetrahydro-1H-pyrrolo[3,2-d]pyrimidin-6-yl)phenoxy]-2N phenylacetamide
2-[4-(2,4-Dioxo-1,3-dipropyl-2,3,4,5-tetrahydro-1H-pyrrolo[3,2-d]pyrimidin-6-yl)phenoxy]-N-(4-fluorophenyl}2-phenylacetamide
6-{4-[2-Oxo-1-phenyl-2-(4-phenylpiperazin-1-yl)ethoxy]phenyl}-1,3-dipropyl-1,5 dihydropyrrolo[3,2-d]pyrimidine-2,4-dione
3-[4-(2,4-Dioxo-1,3-dipropyl-2,3,4,5-tetrahydro-1H-pyrrolo[3,2-pyrimidin-6-yl)phenyl]-N-phenylpropionamide
3-[4-(2,4-Dioxo-1,3-dipropyl-2,3,4,5-tetrahydro-1H-pyrrolo[3,2-d]pyrimidin-6-yl)phenyl]-N-(4-fluorophenyl)propionamide
6-{4-[3-Oxo-3-(4-phenylpiperazin-1-yl)propyl]phenyl}-1,3-dipropyl-1,5-dihydropyrrolo[3,2-d]pyrimidine-2,4-dione
6-{4-[3-(3,4-Dihydro-1H-isoquinolin-2-yl)-3-oxopropyl]phenyl}-1,3-dipropyl-1,5-dihydropyrrolo[3,2-4-pyrimidine-2,4 dione
3-[4-(2,4-Dioxo-1,3 dipropyl-2,3,4,5-tetrahydro-1H-pyrrolo[3,2-d]pyrimidin-6-yl)phenyl]-N-phenylacrylamide
6-{4-[3-Oxo-3-4-phenylpiperazin-1-yl)propenyl]phenyl}-1,3-dipropyl-1,5-dihydropyrrolo[3,2-d]pyrimidine-2,4-dione
6-{4-[3-(3,4-Dihydro-1H-isoquinolin-2-yl)-3-oxo propenyl]phenyl}-1,3-dipropyl-1,5-dihydropyrrolo[3,2-d]pyrimidine-2,4-dione
4-[4-(2,4-Dioxo-1,3-dipropyl-2,3,4,5-tetrahydro-1H-pyrrolo[3,2-d]pyrimidin-6-yl)phenoxy]-M-phenylbutyramide
4-[4-(2,4-Dioxo-1,3-propyl-2,3,4,5-tetrahydro-1H-pyrrolo[3,2-d]pyrimidin-6-yl)phenoxy]-1-(4-fluorophenyl)butyramide
6-{4-[4-Oxo-4-(4-phenylpiperazin-1-yl)butoxy]phenyl}-1,3-dipropyl-1,5-dihydropyrrolo[3,2-d]pyrimidine-2,4-dione
6-{4-[4-4-(3,4-Dihydro-1H-isoquinolin-2-yl) 4-oxobutoxy]phenyl}-1,3-dipropyl-1,5-dihydropyrrolo[3,2-d]pyrimidine-2,4-dione
6-{4-[4-Oxo-4-(6-o-tolyl-2,5-dazabicyclo[2.2.1]hept-2-yl)butoxy]phenyl}-1,3-dipropyl-1,5-dihydropyrrolo[3,2-d]pyrimidine-2,4-dione
4-(2,4-Dioxo-1,3-dipropyl-2,3,4,5-tetrahydro-1H-pyrrolo[3,2-d]pyrimidin-6-yl)-N-phenylbenzamide
4-(2,4-Dioxo-1,3-dipropyl-2,3,4,5-tetrahydro-1H-pyrrolo[3,2-d]pyrimidin-6-yl)-N-(4-fluorophenyl)benzamide
N-(4-Bromophenyl)-4-(2,4-dioxo-1,3-dipropyl-2,3,4,5-tetrahydro-1H-pyrrolo[3,2-d]pyrimidin-6-yl)benzamide
6-[4-(4-Phenylpiperazine-1-carbonyl)phenyl]-1,3-propyl-1,5-dihydropyrrolo[3,2-d]pyrimidine-2,4-dione
6-[4-(3,4-Dihydro-1H-isoquinoline-2-carbonyl)phenyl]-1,3-dipropyl-1,5-dihydropyrrolo[3,2-d]pyrimidine-2,4-dione
6-[4-(3-Phenyl-[1,2,4]oxadiazol-5-ylmethoxy)phenyl]-1,3-dipropyl-1,5-dihydropyrrolo[3,2-d]pyrimidine-2,4-dione
6-{4-[2-oxo-2-{[amino(4-fluorophenyl)methylene diamino]oxy}ethoxy]phenyl}-1,3-dipropyl-1,5-dihydropyrrolo[3,2-d]pyrimidine-2,4-dione
6-{4-[3-(4-Fluorophenyl)-[1,2,4]oxadiazol-5-ylmethoxy]phenyl}-1,3-dipropyl-1,5-dihydropyrrolo[3,2-d]pyrimidine-2-dione
1,3-Dipropyl-6-[4-(3-pyridin-4-yl-[1,2,4]oxadiazol-5-ylmethoxy)phenyl]-1,5-dihydropyrrolo[3,2-d]pyrimidine-2,4-dione
6-[4-(Benzooxazol-2-ylmethoxy)phenyl]-1,3-dipropyl-1,5-dihydropyrrolo[3,2-d)pyrimidine-2,4-dione
6-[4-(5-Phenyl-4,5-dihydrooxazol-2-ylmethoxy)phenyl]-1,3-dipropyl-1,5-dihydropyrrolo[3,2-d]pyrimidine-2,4-dione
6-[4-(4-Methyl-5-phenyl-4,5-dihydrooxazol-2-ylmethoxy)phenyl]-1,3-dipropyl-1,5-dihydropyrrolo[3,2-d]pyrimidine-2,4-dione
6-[4-(7-Benzyl-1-oxa-3,7-diazaspiro(4.5]dec-2-en-2-ylmethoxy)phenyl]-1,3-dipropyl-1,5-dihydropyrrolo[3,2-d]pyrimidine-2,4-dione
1,3-Dipropyl-6-[4-(quinolin-2-ylmethoxy)phenyl]-1,5-dihydropyrrolo[3,2-d]pyrimidine-2,4-dione
2-[4-(2,4-Dioxo-1,3-dipropyl-2,3,4,5-tetrahydro-1H-pyrrolo[3,2-d]pyrimidin-6-yl)phenoxy]-N-pyridin-2-ylacetamide
2-[4-(2,4-Dioxo-1,3-dipropyl-2,3,4,5-tetrahydro-1H-pyrrolo[3,2-d]pyrimidin-6-yl)phenoxy]-N-3-hydroxypyridin-2-yl)acetamide
2-[4-(2,4-Dioxo-1,3-dipropyl-2,3,4,5-tetrahydro-1H-pyrrolo[3,2-d]pyrimidin-6-yl)phenoxy]-N-(5-methylpyridin-2-yl)acetamide
2-[4-(2,4-Dioxo-1,3-dipropyl-2,3,4,5-tetrahydro-1H-pyrrolo[3,2-d]pyrimidin-6-yl)-phenoxy]-N-pyridin-3-ylacetamide
2-[4-(2,4-Dioxo-1,3-dipropyl-2,3,4,5-tetrahydro-1H-pyrrolo[3,2-d]pyrimidin-6-yl)phenoxy]-N-6-methoxypyridin-3-yl)acetamide
2-[4-(2,4-Dioxo-1,3-dipropyl-2,3,4,5-tetrahydro-1H-pyrrolo[3,2-d]pyrimidin-6-yl)phenoxy]-N-pyridin-4-ylmethylacetamide
6-[4-{2-Oxo-2-(4-4-trifluoromethylphenyl)piperazin-1-yl]ethoxy}phenyl)-1,3-dipropyl-1,5-dihydropyrrolo[3,2-]pyrimidine-2,4-dione
6-(4-{2-(4,3-Chlorophenyl)piperazin-1-yl]-2-oxoethoxy}phenyl)-1,3-dipropyl-1,5-dihydropyrrolo[3,2-d]pyrimidine-2,4-dione
2-[4-(2,4-Dioxo-1,3-dipropyl-2,3,4,5-tetrahydro-1H-pyrrolo[3,2-d]pyrimidin-6-yl)phenoxy]-N-pyrazin-2-ylacetamride
N-(2,6-Dimethoxypyrimidin-4-yl)-2-[4-(2,4-dioxo-1,3-dipropyl-2,3,4,5-tetrahydro-1H-pyrrolo[3,2-d]pyrimidin-6-yl)phenoxy]acetamide
6-{4-[2-(3-Aminopyrazol-1-yl)-2-oxoethoxy]phenyl}-1,3-dipropyl-1,5-dihydro pyrrolo[3,2-d]pyrimidine-2,4-dione
6-(4-{2-[4-3-Chlorophenyl)piperazin-1-yl]-2-oxoethoxy}phenyl)-1,3-dimethyl-1,5-dihydropyrrolo[3,2-d]pyrimidine-2,4-dione
1,3-Dimethyl-6-(4-{2-oxo-2-[4-(4-trifluoromethylphenyl)piperazin-1-yl]ethoxy}phenyl)-1,5-hydropyrrolo[3,2-d]pyrimidine-2,4-dione
6-(4-{2-[4-(4-Bromophenyl)piperazin 1-yl]-2-oxoethoxy}phenyl)-1,3-dimethyl-1,5-dihydropyrrolo[3,2-d]pyrimidine-2,4-dione
6-{4-[2-(4-Hydroxy-4-phenylpiperidin-1-yl)-2-oxoethoxy]phenyl}-1,3-dimethyl-1,5-dihydropyrrolo[3,2-d]pyrimidine-2,4-dione
1-{2-[4-(1,3-Dimethyl-2,4-dioxo-2,3,4,5-tetrahydro-1H-pyrrolo [3,2-d]pyrimidin-6-yl)phenoxy]acetyl})phenylpiperidine-4-carbonitrile
6-{4-[2-(4,4-Diphenylpiperidin-1-yl)₂-oxoethoxy]phenyl}-1,3-dimethyl-1,5-dihydropyrrolo[3,2-d]pyrimidine-2,4-dione
6-(4-{2-[4-(4-Chlorophenyl)-4-hydroxypiperidin-1-yl]-2-oxoethoxy}phenyl)-1,3-dimethyl-1,5-dihydropyrrolo[3,2-d]pyrimidine-2,4-dione
6-(4-{2-[4-(3,5-Dichloropyridin-4-yl)piperazin-1-yl]-2-oxoethoxy}phenyl)-1,3-dimethyl-1,5-dihydropyrrolo[3,2-d]pyrimidine-2,4-dione
6-(4-{2-[4-(5-Chlorobenzothiazol-2-yl)piperazin-1-yl]-2-oxoethoxy}phenyl)-1,3-dimethyl-1,5-dihydropyrrolo[3,2-d]pyrimidine-2,4-dione
1,3-Dimethyl-6-{4-[2-oxo-2-(1,3,4,9-tetrahydro-b-carbolin-2-yl)ethoxy]phenyl}-1,5-dihydropyrrolo[3,2-d]pyrimidine-2,4-6-dione
6-[4-(2-{4-[1-(4-Fluorophenyl)methanoyl]piperidin-1-yl}-2-oxo-ethoxy)phenyl]-1,3-dimethyl-1,5-dihydropyrrolo[3,2-d]pyrimidine-2,4-dione
2-[4-(1,3-Dimethyl-2,4-dioxo-2,3,4,5-tetrahydro-1H-pyrrolo[3,2-d]pyrimridin-6-yl)-phenoxy]-N-pyridin-4-ylmethylacetamide
4-{2-[4-(1,3-Dimethyl-2,4-dioxo-2,3,4,5-tetrahydro-1H-pyrrolo[3,2-d]pyrimidin-6-yl)phenoxy]ethanoyl}piperazine-1-carboxylic acid ethyl ester
6-(4-{2-[4-(2-Methoxyphenyl)piperidin-1-yl]-2-oxoethoxy}phenyl)-1-methyl-3-propyl-1,5-dihydropyrrolo[3,2-d]pyrimidine-2,4-dione
6-(4-{2-[4-(3,5-Dichloropyridin-4-yl)piperazin-1-yl]-2-oxoethoxy}phenyl)-1-methyl-3-propyl-1,5-dihydropyrrolo[3,2-d]pyrimidine-2,4-dione
N-(6-Methoxypyridin-3-yl)-2-[4-(1-methyl-2,4-dioxo-3-propyl-2,3,4,5-tetrahydro-1H-pyrrolo[3,2-d]pyrimidin-6-yl)phenoxy]acetamide
1-Methyl-6-(4-{2-oxo-2-[4-(4-trifluoromethylphenyl)piperazin-1-yl]ethoxy}phenyl)-3-propyl-1,5-dihydropyrrolo[3,2-d]pyrimidine-2,4-dione
6-[4-(2-Morpholin-4-yl-2-oxoethoxy)phenyl]-3-propyl-1,5-dihydropyrrolo[3,2-d]pyrimidine-2,4-dione
6-{4-[2-(4-Methylpiperazin-1-yl)-2-oxoethoxy]phenyl}-3-propyl-1,5-dihydropyrrolo[3,2-d]pyrimidine-2,4-dione
2-[4-(2,4-Dioxo-3-propyl-2,3,4,5-tetrahydro-1H-pyrrolo[3,2-d]pyrimidin-6-yl)phenoxy]-N-2-hydroxyethyl)acetamide
6-(4-{2-[4-(2-Methoxyphenyl)piperazin-1-yl]-2-oxoethoxy}phenyl)-3-propyl-1,5 dihydropyrrolo[3,2-d]pyrimidine-2,4-dione
6-{4-[2-(4-Benzylpiperazin-1-yl)-2-oxoethoxy]phenyl}-3-propyl-1,5-dihydropyrrolo[3,2-d]pyrimidine-2,4-dione
2-[4-2,4-Dioxo-3-propyl-2,3,4,5-tetrahydro-1H-pyrrolo[3,2-d]pyrimidin-6-yl)-phenoxy]-N-(4-fluorophenyl)acetamide
N-(4-Bromophenyl)-2-[4-(2,4-dioxo-3-propyl-2,3,4,5-tetrahydro-1H-pyrrolo[3,2-d]pyrimidin-6-yl)phenoxy]acetamide
6-{4-[2-Oxo-2-4-phenylpiperazin-1-yl)ethoxy]phenyl}-1-propyl-1,5-dihydropyrrolo[3,2-]pyridine-2,4-dione
6-(4-{2-[4-(4-Fluorophenyl)piperazin-1-yl]-2-oxo-ethoxy}phenyl)-3-methyl-1-(3-morpholin-4-ylpropyl)-1,5-dihydro-pyrrolo[3,2-d]pyrimidine-2,4-dione
3-Methyl-1-(3-morpholin-4-yl-propyl)-6-{4-[2-oxo-2-(4-phenyl-piperazin-1-yl)ethoxy]phenyl}-1,5-dihydropyrrolo[3,2-d]pyrimidine-2,4-dione
3-Methyl-1-(3-moropholin-4-yl-propyl)-6-(4-{2-oxo-2-[4-(4-trifluoromethyl-phenyl)-piperazin-1-yl]-ethoxy}phenyl)-1,5-dihydro-pyrrolo[3,2-d]pyrimidine-2,4-dione
Pyrazin-2-yl-carbamic acid 4-(2,4-dioxo-1,3-dipropyl-2,3,4,5-tetrahydro-1H-pyrrolo[3,2-d]pyrimidin-6-yl)benzyl ester
(2,6-Dimethoxy-pyrimidin 4-yl)-carbamic acid 4-(2,4-dioxo-1,3-dipropyl-2,3,4,5-tetrahydro-1H-pyrrolo[3,2-d]pyrimidin-6-yl)benzyl ester
Pyridin-4-ylmethyl carbamic acid 4-(2,4-dioxo-1,3-dipropyl-2,3,4,5-tetrahydro-1H-pyrrolo[3,2-d]pyrimidin-6-yl)benzyl ester
4-(3-Chlorophenyl)piperazine-1-carboxylic acid 4-(2,4-dioxo-1,3-dipropyl-2,3,4,5-tetrahydro-1H-pyrrolo[3,2-d]pyrimidin-6-yl)benzyl ester
(1H-Pyrazol-3-yl)carbamic acid 4-(2,4-dioxo-1,3-dipropyl-2,3,4,5-tetrahydro-1H-pyrrolo[3,2-d]pyrimidin-6-yl)benzyl ester
4-(3-Trifluoromethylphenyl)piperazine-1-carboxylic acid 4-(2,4-dioxo-1,3-dipropyl-2,3,4,5-tetrahydro-1H-pyrrolo[3,2-d]pyrimidin-6-yl)benzyl ester
Isoxazol-3-yl-carbamic acid 4-(2,4-dioxo-1,3-dipropyl-2,3,4,5-tetrahydro-1H-pyrrolo[3,2-d]pyrimidin-6-yl)benzyl ester
(4-Fluorophenyl)-carbamic acid 4-(1,3-dimethyl-2,4-dioxo-2,3,4,5-tetrahydro-1H-pyrrolo[3,2-d]pyrimidin-6-yl)benzyl ester
Benzylcarbamic acid 4-(1,3-dimethyl-2,4-dioxo-2,3,4,5-tetrahydro-1H-pyrrolo[3,2-d]pyrimidin-6-yl)benzyl ester
Phenylcarbamic acid 4-(1,3-dimethyl-2,4-dioxo-2,3,4,5-tetrahydro-1H-pyrrolo[3,2-d]pyrimidin-6-yl)benzyl ester
Pyridin-2-yl-carbamic acid 4-(1,3-dimethyl-2,4-dioxo-2,3,4,5-tetrahydro-1H-pyrrolo[3,2-d]pyrimidin-6-yl)benzyl ester
(5-Methylpyridin-2-yl)-carbamic acid 4-(1,3-dimethyl-2,4-dioxo-2,3,4,5-tetrahydro-1H-pyrrolo[3,2-d]pyrimidin-6-yl)benzyl ester:
Thiophen-2-yl-carbamic acid 4-(1,3-dimethyl-2,4-dioxo-2,3,4,5-tetrahydro-1H-pyrrolo[3,2-d]pyrimidin-6-yl)-benzyl ester
Thiophen-3-yl-carbamic acid 4-(1,3-dimethyl-2,4-dioxo-2,3,4,5-tetrahydro-1H-pyrrolo[3,2-d]pyrimidin-6-yl)-benzyl ester
Furan-2-yl-carbamic acid 4-(1,3-dimethyl-2,4-dioxo-2,3,4,5-tetrahydro 1H-pyrrolo[3,2-d]pyrimidin-6-yl)-benzyl ester
4-Phenylpiperazine-1-carboxylic acid 4-(1,3-dimethyl-2,4-dioxo-2,3,4,5-tetrahydro-1H-pyrrolo[3,2-d]pyrimidin-6-yl)benzyl ester
3,4-Dihydro-1H-isoquinoline-2-carboxylic acid 4-(1,3-dimethyl-2,4-dioxo-2,3,4,5-tetrahydro-1H-pyrrolo[3,2-d]pyrimidin-6-yl)benzyl ester
Thiophen-2-yl-carbamic acid 2-[4-(2,4-dioxo-1,3-dipropyl-2,3,4,5-tetrahydro-1H-pyrrolo[3,2-d]pyrimidin-6-yl)phenoxy]ethyl ester
(4-Bromophenyl)carbamic acid 2-[4-(2,4-dioxo-1,3-dipropyl-2,3,4,5-tetrahydro-1H-pyrrolo[3,2-d]pyrimidin-6-yl)phenoxy]ethyl ester
1-[1-(2,6-Difluoro-phenyl)methanoyl]-3-[4-(2,4-dioxo-1,3-dipropyl-2,3,4,5-tetrahydro-1H-pyrrolo[3,2-d]pyrimidin-6-yl)benzyl]urea
6-[4-(5-Fluorobenzooxazol-2-ylmethoxy)phenyl]-1,3-dipropyl-1,5-dihydropyrrolo[3,2-d]pyrimidine-2;4-dione
6-[4-(1H-Benzoimidazol-2-ylmethoxy)phenyl]-1,3 dipropyl-1,5-dihydropyrrolo[3,2-d]pyrimidine-2,4-dione
1,3-Dimethyl-6-[4-(quinolin-2-ylmethoxy)phenyl]-1,5-dihydropyrrolo[3,2-d]pyrimidine-2,4-dione
1,3-Dimethyl-6-[4-(3-phenyl[1,2,4]oxadiazol-5-ylmethoxy)phenyl]-1,5-dihydropyrrolo[3,2-d]pyrimidine-2,4-dione
1-Methyl-6-[4-(3-phenyl[1,2,4]oxadiazol-5-ylmethoxy)phenyl]-3-propyl-1,5-dihydropyrrolo[3,2-d]pyrimidine-2,4-dione
6-{4-[3-(4-Fluorophenyl)[1,2,4]oxadiazol-5-ylmethoxy]phenyl}-1-methyl-3-propyl-1,5-dihydropyrrolo[3,2-d]pyrimidine-2,4-dione
6-[4-(5-Chlorobenzooxazol-2-ylmethoxy)phenyl]-1-methyl-3-propyl-1,5-dihydropyrrolo[3,2-d]pyrimidine-2,4-dione
6-{4-[3-(4-Bromophenyl)-[1,2,4]oxadiazol-5-ylmethoxy]-phenyl}-3-propyl-1,5-dihydropyrrolo[3,2-d]pyrimidine-2,4-dione
1,3-Dimethyl-6-{4-[1-(3-phenyl[1,2,4]oxadiazol-5-yl)ethoxy]phenyl}-1,5-dihydropyrrolo[3,2-d]pyrimidine-2,4-dione
6-(4-{1-[3-(4-Fluorophenyl)[1,2,4]oxadiazol-5-yl]ethoxy}phenyl)-1,3-dimethyl-1,5-dihydropyrrolo[3,2-d]pyrimidine-2,4-dione
1,3-Dimethyl-6-{4-[1-(3-thiophen-3-yl[1,2,4]oxadiazol-5-yl)ethoxy]phenyl 1,5-dihydropyrrolo[3,2-d]pyrimidine-2,4-dione
1,3-Dimethyl-6-(4-{1-[3-(4-methylsulfanylphenyl)[1,2,4]oxadiazol-5-yl]ethoxy}phenyl) 1,5-dihydropyrrolo[3,2-d]pyrimidine-2,4-dione
6-{4-[(4-Bromophenylamino)methyl]phenyl}-1,3-dipropyl-1,5-dihydropyrrolo[3,2-d]pyrimidine-2,4-dione
6-(4-Phenylaminomethylphenyl)-1,3-dipropyl-1,5-dihydropyrrblo[3,2-d]pyrimidine-2,4-dione
   and pharmaceutically acceptable salts thereof.

Of outstanding interest are 6-phenylpyrrolopyrimidinedione-derivatives of formula (I), and pharmaceutically acceptable salts thereof, wherein:
R¹ and R² are the same or different and each independently represent a C₁-C₄ alkyl group;
R³ represents hydrogen or halogen;
R⁴ and R⁵ are the same or different and each independently represent hydrogen, C₁-C₄ alkyl, C₁-C₄ alkoxy or C₁-C₄ alkylthio;
L₁ is —O—CH₂—, —CH₂—O— or —CH₂NH—, for example —O—CH₂—; and
R⁶ represents a phenyl group; an oxadiazolyl group which is unsubstituted or substituted by a phenyl group; or a group of formula —C(O)NR¹⁰R¹¹, wherein either R¹⁰ is hydrogen and R¹¹ is a thienyl group, a thiadiazolyl group, a pyridyl group, an optionally substituted phenylcarbonyl group or a phenyl group which is unsubstituted or substituted by 1 or 2 substituents selected from halogen atoms and phenyl and benzyloxy groups or R¹⁰ and R¹¹ form, together with the N atom to which they are attached, a 1, 2, 3, 4-tetrahydroisoquinoline group, a 1,3,4,9-tetrahydro-beta-carbolinyl group, a piperidinyl group or a piperazinyl group, the piperidinyl and piperazinyl groups being unsubstituted or substituted by 1 or 2 groups selected from hydroxy, optionally substituted phenyl and optionally substituted pyridyl.

In this embodiment, R⁶ may represent, for example, —C(O)NR¹⁰R¹¹ or an oxadiazolyl group which is unsubstituted or substituted by a phenyl group, wherein either R¹⁰ is hydrogen and R¹¹ is a thiadiazolyl group, a pyridyl group or a phenyl group which is unsubstituted or substituted by 1 or 2 substituents selected from halogen atoms and phenyl and benzyloxy groups or R¹⁰ and R¹¹ form, together with the N atom to which they are attached, a 1, 2, 3, 4-tetrahydroisoquinoline group, a piperidinyl group or a piperazinyl group, the piperidinyl and piperazinyl groups being unsubstituted or substituted by 1 or 2 phenyl groups.

Examples of such compounds include:
{4-[2-Oxo-2-(4-phenylpiperazin-yl)ethoxy]phenyl}-1,3-dipropyl-1,5-dihydropyrrolo[3,2-d]pyrimidine-2,4-dione
6-([2-3,4-Dihydro 1H-isoquinolin-2-yl)-2-oxoethoxy]phenyl}-1,3 dipropyl-1,5-dihydropyrrolo[3,2-d]pyrimidine-2,4-dione
2-[4-(1,3-Dimethyl-2,4-dioxo-2,3,4,5-tetrahydro-1H-pyrrolo[3,2-d]pyrimidin-6-yl)phenoxy]-N-(4-fluorophenyl)acetamide
1,3-Dimethyl-6-{4-(2-oxo-2-(4-phenylpiperazin-1-yl)ethoxy]phenyl}-1,5-dihydropyrrolo[3,2-d]pyrimidine-2,4-dione
N-Biphenyl-4-yl-2-[4-(1-methyl-2,4 dioxo-3-propyl-2,3,4,5-tetrahydro-1H-pyrrolo[3,2-d]pyrimidin-6-yl)phenoxy]acetamide
6-{4-[2-(4,4-Diphenylpiperidin-1-yl)₂-oxo-ethoxy]phenyl)-1-methyl-3-propyl-1,5-dihydropyrrolo[3,2-d]pyrimidine-2,4-dione
6-[4-(3-Phenyl-[1,2,4]oxadiazol-5-ylmethoxy)phenyl]-1,3-dipropyl-1,5-dihydropyrrolo[3,2-d pyrimidine-2,4-dione
N-(4-Bromophenyl)-2-[4-(1,3-dimethyl-2,4-dioxo-2,3,4,5-tetrahydro-1H-pyrrolo[3,2-d]pyrimidin-6-yl)-3-methoxyphenoxy]acetamide
2-(4-(2,4-Dioxo-1,3-dipropyl-2,3,4,5-tetrahydro-1H-pyrrolo[3,2-d]pyrimidin-6-yl)phenoxy]-N-(4-fluorophenyl)acetamide
2-[4-(1,3-Dimethyl-2,4-dioxo-2,3,4,5-tetrahydro-1H-pyrrolo[3,24-pyrimidin-6-yl)phenoxy]-N-[1,3,4]thiadiazol-2-ylacetamide
2-(4-(7-Bromo-2,4-dioxo-1,3-dipropyl-2,3,4,5-tetrahydro-1H-pyrrolo[3,2-d]pyrimidin-6-yl)phenoxy]-N-4-fluorophenyl)acetamide
N-(4-Benzyloxyphenyl)-2-[4-(2,4-dioxo-1,3-dipropyl-2,3,4,5-tetrahydro 1H-pyrrolo[3,2-d]pyrimidin-6-yl)phenoxy]acetamide
2-[4-(1,3-Dimethyl-2,4 dioxo-2,3,4,5-tetrahydro-1H-pyrrolo[3,2-d]pyrimidin-6-yl)-2-methoxyphenoxy]-N-(4-fluorophenyl)acetamide
Thiophen-3-yl-carbamic acid 4-(1,3-dimethyl-2,4-dioxo-2,3,4,5-tetrahydro-1H-pyrrolo[3,2-d]pyrimidin-6-yl)benzyl ester
6-(4-{2-[4-(4-Chlorophenyl)-4-hydroxypiperidin-1-yl]-2-oxoethoxy}phenyl)-1,3 dimethyl-1,5-dihydropyrrolo[3,2-d]pyrimidine-2,4-dione
6-(4-{2-[4-(3,5-Dichloropyridin-4-yl)piperazin-1-yl]-2-oxoethoxy}phenyl)-1-methyl-3-propyl-1,5-dihydropyrrolo[3,2-d]pyrimidine-2,4-dione
1,3-Dimethyl-6-{4-[2-oxo-2-(1,3,4,9-tetrahydro-b-carbolin-2-yl)ethoxy]phenyl}-1,5-dihydropyrrolo[3,2-d]pyrimidine-2,4-dione
1-[1-(2,6-Difluorophenyl)methanoyl]-3-[4-(2,4-dioxo-1,3-dipropyl-2,3,4,5-tetrahydro-1H-pyrrolo[3,2-d]pyrimidin-6-yl)benzyl]urea
6-(4-Phenylaminomethylphenyl)-1,3-dipropyl-1,5 dihydropyrrolo[3,2-d]pyrimidine-2,4-dione
   and pharmaceutically acceptable salts thereof.

According to a further feature of the present invention, the 6-phenyl-1,5-dihydropyrrolo[3,2-d]pyrimidine-2,4-dione derivatives of general formula (I) in which R⁶ is CONR¹⁰R¹¹ can be prepared by reaction of the corresponding carboxylic acids of formula (II): 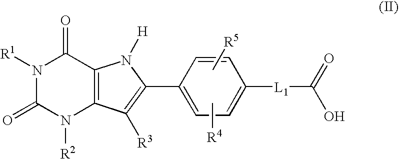
(wherein R¹, R², R³, R⁴, R⁵, and L₁ are as hereinbefore defined) and the corresponding amines (III): 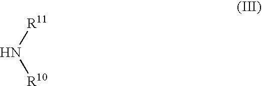
(wherein R¹⁰ and R¹¹ are as hereinbefore defined). The reaction is carried out in an organic solvent, preferably a polar aprotic organic solvent such-as dichloromethane, N,N-dimethylformamide or tetrahydrofuran, at a temperature from 10° C. to 60° C. and in the presence of an organic base, preferably an amine base such as triethylamine or polymer, supported morpholine, and in the presence of standard coupling agents such as 1-hydroxybenzotriazole and 1-[3-(dimethylamino)propyl]-3-ethylcarbodiimide hydrochloride.

The thus obtained compound of formula (I) can be converted to a further compound of formula (I) by standard functional group interconversions known to those of skill in the art. Thus, for example, in the case that R³ is chlorine or bromine, the carboxylic acid of formula (II) is obtained from the compound of formula (II) where R¹ is hydrogen by chlorination or bromination using methods known per se.

The 6-phenylpyrrolopyrimidinedione derivatives of general formula (I) are also prepared from vinyl derivatives (IV) (wherein R¹, R², R⁴, R⁵, and L, are as hereinbefore defined) and amines (III) using the coupling procedure described below and subsequent reductive cyclization mediated by triethyl phosphite or sodium dithionite in formic acid both at reflux temperature.

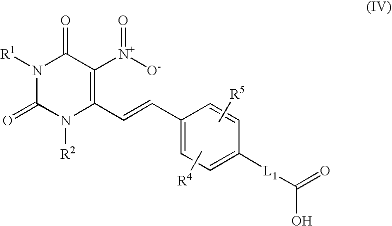

When R⁶ is a said group of formula (H), wherein X, Y¹ and Y² are as hereinbefore defined, the ring of R⁶ is prepared from carboxylic acid (II) and amines (V) or amide derivatives (VI) by amide type coupling followed by cyclodehydration typically performed in toluene with catalytic amounts of acid or in dichloromethane or tetrahydrofuran using dehydration agents (such as SOCl₂ POCl₃, Burgess reagent or polyphosphoric acid) and in the products derived from amine (V) a further oxidation can be done, typically performed by NiO₂ or MnO₂.

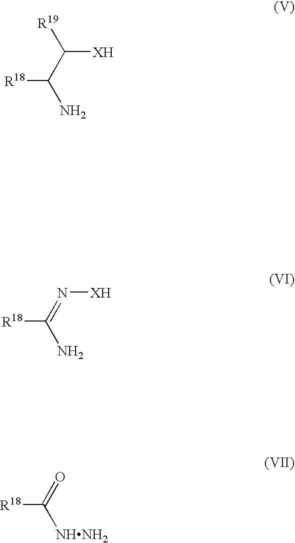

The 6-phenylpyrrolopyrimidinedione derivatives of general formula (II) are prepared from vinyl derivatives (IV) by reductive cyclization using the methods described hereinbefore.

The vinyl derivatives of general formula (TV) are prepared by reaction of the corresponding 6-methyl-5-nitrouracils (VIII): 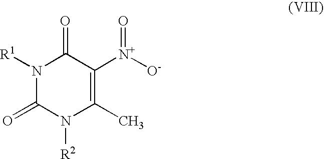
(wherein R¹ and R² are as hereinbefore defined), and the corresponding benzaldehydes (IX): 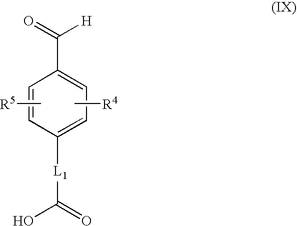
(wherein L₁, R⁴ and R⁵ are as hereinbefore defined) by methods known per se, e.g. C. E. Müller et al., J. Med. Chem. 1994, 37, 1526-1534 and references cited therein.

When R⁶ is —ON═CR¹²R¹³, the products of general formula (I) are prepared by reacting a carboxylic acid of formula (II) with a compound of formula R¹²—C(R¹³)═N—OH using standard coupling procedures known in the art.

When R⁶ is —S(O)₂—NR¹⁰R¹¹, aryl, heterocyclyl or heteroaryl the products of general formula (I) are prepared by condensation of the 6-methyl-5-nitrouracils (VIII) with the corresponding benzaldehydes (X) to give the vinyl derivatives, followed by reductive cyclization as in the preparation of compounds of general formula (II).

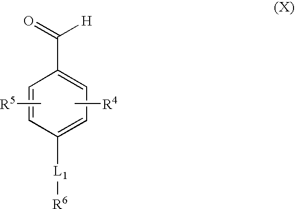

When L₁ is —(CR⁸R⁹)ₘO—, —O(CR⁸R⁹)ₘO or —(CR⁸R⁹)ₘN(Z)- the products of general formula (1) are prepared by condensation of the alcohols (XI), (XII) or amine (XIII) with the corresponding isocianates to give the carbamate or urea derivatives.

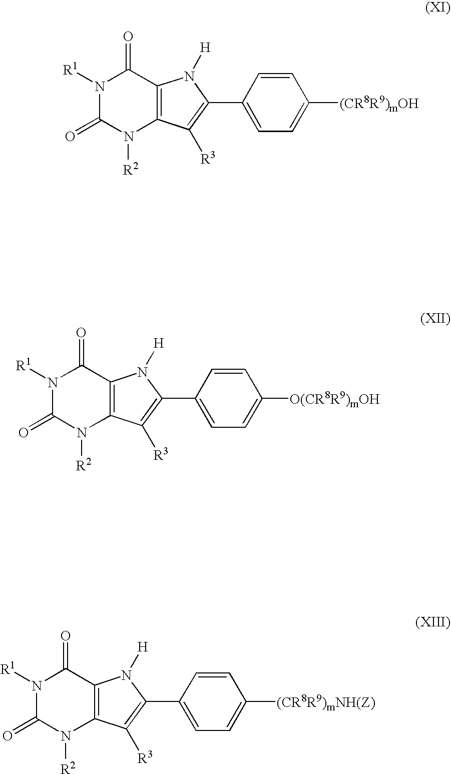

Compounds (XI) and (XII) are prepared by reduction of the carboxylic acid of general formula (II) wherein L₁ is —(CR⁸R⁹)ₘ₋₁— or —(CR⁸R⁹)ₘ₋₁— using standard reductive agents such as borane or aluminium hydrides in common organic solvents such as tetrahydrofuran at a temperature from 0° C. to 100° C.:

Compounds of general formula (M) can be obtained from alcohols (XI) by using standard procedures known in the art.

The 6-methyl-5-nitrouracils. (VIII) can be prepared from the corresponding N,N′-disubstituted ureas by methods known per se, e.g. S. Senda et al., J. Med. Chem 1972, 15, 471476 or H. Egg Synthesis 1982, 1071-1072 and references cited therein. The compounds of formulae (III), (V), (VI), (VII), (VIII), (IX) and (X) are known compounds or may be prepared by analogy with known methods. The compounds of formula R²—C(R¹³)═N—OH are commercially available or may be prepared by analogy with known methods.

The 6-phenyl-1,5-dihydropyrrolo[3,2-d]pyrimidine-2,4-dione derivatives of formula (I) in which there is the presence of a basic group can be converted by methods known per se into pharmaceutically acceptable salts, preferably acid addition salts by treatment with organic or inorganic acids such as fumaric, tartaric, succinic or hydrochloric acid. Also 6-phenyl-1,5-dihydropyrrolo[3,2-d]pyrimidine-2,4-dione derivatives of formula (1) in which there is the presence of an acidic group, may be converted into pharmacologically acceptable salts by reaction with an alkali metal hydroxide such as sodium or potassium hydroxide or an organic base such as diethanolamine. The acid or alkali addition salts so formed may be interchanged with suitable pharmaceutically acceptable counter ions using processes known per se.

Adenosine 2b Receptor Subtype Competition Radioligand Binding

Human membranes from recombinant A2b receptors were purchased from Receptor Biology, Inc. (USA).

Competition assays were carried out by incubation of membranes from hA2b receptors transfected to HEK293 cells, [³H]DPCPX as radioligand, buffer (50 mM Tris-HCl (pH 6.5), 10 mM MgCl₂, 1 mM EDTA, 0.1 mM benzamidine, 2 units/ml adenosine deaminase), and unlabelled ligand in a total volume of 0.1 ml for 30 min at 25° C. NECA was used to determinate non-specific binding. Filter over Schleicher&Schuell GF/52 filters (pre-soaked 0.5% polyethylenyimine) in a Brandel cell harvester. Unbound radioligand was removed with 4×2 ml ice-cold 50 mM Tris-Hcl 50 mM (pH 6.5).

Adenosine 2a Receptor Subtype Competition Radioligand Binding

Human membranes from recombinant A2a receptors were purchased from Receptor Biology, Inc. (USA).

Competition assays were carried out by incubation of membranes from hA2a receptors transfected to HEK293 cells, [³H]ZM241385 as radioligand, buffer (50 mM Tris-HCl (pH 7.4), 10 mM MgCl₂, 1 mM EDTA, 2 units/ml adenosine deaminase), and unlabelled ligand in a total volume of 0.2 ml for 90 min at 25° C. NECA was used to determinate non-specific binding. Filter over Schleicher&Schuell GF/52 filters (pre-soaked 0.5% polyethylenyimine) in a Brandel cell harvester. Unbound radioligand was removed with 3×3 ml ice-cold 50 mM Tris-Hcl 50 mM (pH 7.4), 0.9% NaCl.

The results are shown in Table 1 and Table 2.

TABLE 1

| Example | IC₅₀ A2b (nM) |
|---|---|
| 2 | 7 |
| 4 | 3 |
| 58 | 5 |
| 68 | 8 |
| 99 | 9 |
| 100 | 10 |
| 210 | 17 |
| 156 | 6 |
| 3 | 5 |
| 67 | 24 |

It can be seen from Table 1 that the compounds of formula (I) are potent inhibitors of the A2b adenosine receptor subtype. Preferred 6-phenyl-1,5-dihydropyrrolo [3,2-d]pyrimidine-2,4-dione derivatives of the invention possess an IC₅₀ value for the inhibition of A2b (determined as defined above) of less than 50 nM, preferably less than 10 nM and most preferably less than 5 nM.

TABLE 2

| Example | IC₅₀ A2a (nM) |
|---|---|
| 3 | 22 |
| 67 | 26 |
| 138 | 38 |
| 26 | 42 |
| 160 | 84 |

It can be seen from Table 2 that the compounds of formula (I) are potent inhibitors of the A2a adenosine receptor subtype. Some preferred 6-phenyl-1,5-dihydro pyrrolo[3,2-d]pyrimidine-2,4-one derivatives of the invention possess an IC₅₀ value for the inhibition of A2a (determined as defined above) of less than 100 nM, preferably less than 50 nM and most preferably less than 10 nM.

The 6-phenyl-1,5-dihydropyrrolo[3,2-d]pyrimidine-2,4-dione derivatives of the invention are useful in the treatment or prevention of asthma, bronchoconstriction, allergic potentiation, inflammation or reperfusion injury, myocardial ischemia, inflammation, diarrheal diseases, brain arteriole diameter constriction, Parkinson's disease, non insulin dependent diabetes mellitus, release of allergic mediators, and/or treatment of an autoimmune diseases. Examples of autoimmune diseases which can be treated or prevented using the compounds of the invention are Addison's disease, autoimmune hemolytic anemia, Crohn's disease, Goodpasture's syndrome, Grave's disease, Hashimoto's thyroiditis, idiopathic thrombocytopinic purpura, insulin-dependent diabetes mellitus, multiple sclerosis, myasthenia gravis, pemphigus vulgaris, pernicious anemia, poststreptococcal glomerulonephritis, psoriasis, rheumatoid arthritis, scleroderma, Sjogren's syndrome, spontaneous infertility, and syntemic lupus erythematosus.

Accordingly, the 6-phenyl-1,5-dihydropyrrolo[3,2-d]pyrimidine-2,4-dione derivatives of the invention and pharmaceutically acceptable salts thereof, and pharmaceutical compositions comprising such compound and/or salts thereof, may be used in a method of treatment of disorders of the human body which comprises administering to a patient requiring such treatment an effective amount of a 6-phenyl-1,5 dihydropyrrolo[3,2-d]pyrimidine-2,4-dione derivative of the invention or a pharmaceutically acceptable salt thereof.

The present invention also provides pharmaceutical compositions which comprise, as an active ingredient, at least a 6-phenyl-1,5-dihydropyrrolo[3,2-d]pyrimidine-2,4-dione derivative of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight of the composition depending upon the nature of the formulation and whether further dilution is to be made prior to application. Preferably the compositions are made up in a form suitable for oral, topical nasal, rectal, percutaneous or injectable administration.

The pharmaceutically acceptable excipients which are admixed with the active compound, or salts of such compound, to form the compositions of this invention are well-known per se and the actual excipients used depend inter alia on the intended method of administering the compositions.

Compositions of this invention are preferably adapted for injectable and per os administration. In this case, the compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate-parenteral injection fluid.

Effective doses are normally in the range of 2-2000 mg of active ingredient per day. Daily dosage may be administered in one or more treatments, preferably from 1 to 4 treatments, per day.

The syntheses of the compounds of the invention and of the intermediates for use therein are illustrated by the following Examples (including Preparation Examples (Preparations 1-26)) which do not limit the scope of the invention in any way.

¹H Nuclear Magnetic Resonance Spectra were recorded on a Varian Gemini 300 spectrometer. Melting points were recorded using a Perkin Elmer DSC-7 apparatus. The chromatographic separations were obtained using a Waters 2690 system equipped with a Symmetry C18 (2.1×10 mm, 3.5 μM) column As detectors a Micromass I) mass spectrometer using ES ionization and a Waters 996 Diode Array detector were used The mobile phase was formic acid (0.46 mL), ammonia (0.115 mL) and water (1000 mL) (A) and formic acid (0.4 mL), ammonia (0.1 mL), methanol (500 mL), and acetonitrile (500 mL) (B): initially from 0% to 95% of B in 20 min, and then 4 min. with 95% of B. The reequilibration time between two injections was 5 min. The flow rate was 0.4 mL/min. The injection volume was 5 μL. Diode array chromatograms were processed at 210 nm.

### PREPARATION EXAMPLES

Preparation 1

### {4-[2-(5-Nitro-2,6-dioxo-1,3-dipropyl-1,2,3,6-tetrahydropyrimidin-4-yl)vinyl]phenoxy}acetic Acid

To a solution of 6-methyl-5-nitro-1,3-dipropyl-1H-pyrimidine-2,4-dione (4.1 g, 16.08 mmol) in dry dioxane (52 mL) was added piperidine (1.6 mL, 1835 mmol) and (4-formylphenoxy)acetic acid (2.9 g, 16.08 mmol). The mixture was stirred at reflux temperature for 68 hours. The resulting solution was concentrated under vacuum and the residue was treated with ethanol (I 00 mL) until formation of a precipitate was observed The solid was collected by filtration and dried under vacuum to yield the title product (4.8 g, 72%) as a yellow solid.

m.p.(H₂O): 72-74° C.

δ¹H NMR (DMSO): 10.10 (bs, 1H), 7.61 (d, 2H), 6.99 (m, 4H), 4.76 (s, 2H), 3.84 (m, 4H), 1.61 (m, 4H), 0.87 (m, 6H).

ESI/MS (m/e, %): 418 [(M+1)⁺, 100].

Preparation 2

### [4-(2,4-Dioxo-1,3-dipropyl-2,3,4,5-tetrahydro-1H-pyrrolo[3,2-d]pyrimidin-6-yl)phenoxy]acetic Acid

a) A solution of 6-methyl-5-nitro-1,3-dipropyl-1H-pyrimidine-2,4-dione (7.72 g, 30.24 mmol), (4-formylphenoxy)acetic acid (6 g, 33.26 mmol) and piperidine (4.5 mL, 45.36 mmol) in ethanol (140 mL) with 3A molecular sieves (9.8 g) was refluxed for 5 hours. The resulting suspension was diluted with dichloromethane (75 mL), filtrated and the filtrates were evaporated under reduced pressure. The residue was suspended in water (100 mL) and acetic acid was added until pH was slightly acidic. The aqueous suspension was partitioned between dichloromethane and brine, then the organic phase was separated, washed with 2N HCL, brine, dried (MgSO₄) and evaporated under reduced pressure. The residue was triturated with a mixture of ethyl ether and isopropyl ether. The precipitate was collected by filtration and dried under vacuum to yield the compound of Preparation 1 (8.08 g, 64%).

b) To a stirred solution of the above compound (8.08 g, 19.36 mmol) in formic acid (180 mL) was slowly added sodium dithionite (19.8 g, 96.8 mmol) and the mixture was refluxed overnight The resulting solution was cooled to room temperature and poured into water (750 mL). The precipitate was collected by filtration and washed with water and ethyl ether, then dried under vacuum to yield [4-(2,4-Dioxo-1,3-dipropyl-2,3,4,5-tetrahydro-1H-pyrrolo[3,2-d]pyrimidin-6-yl)phenoxy]acetic acid 5.6 g, 75%) as a white solid.

m.p.(MeOH/H₂O): 280-282° C.

δ ¹H NMR (DMSO): 7.85 (d, 2H), 6.98 (d, 2H), 6.64 (d, 1H), 4.74 (s, 2H), 3.87 (m, 4H), 1.62 (m, 4H), 0.90 (m, 6H).

ESI/MS (m/e, %): 386 [(M+1)⁺, 100].

Preparation 3

### [4-(1,3-Dimethyl-2,4-dioxo-2,3,4,5-tetrahydro-1H-pyrrolo[3,2-d]pyrimidin-6-yl)phenoxy]acetic Acid Ethyl Ester

a) Following the same procedure as in Preparation 1, from 1,3,6-trimethyl-5-nitro-1H-pyrimidine-2,4-dione (2.47 g, 12.4 mmol) and (4-formylphenoxy)acetic acid ethyl ester (2.58 g, 12.4 mmol), {4-[2-(1,3-Dimethyl-5-nitro-2,6 dioxo-1,2,3,6-tetrahydropyrimidin-4-yl)vinyl]phenoxy}acetic acid ethyl ester was obtained (2.4, 50%) as a yellow solid.

m.p.(EtOH): 136-138° C.

δ ¹H NMR (CDCl₃): 7.43 (d, 2H), 7.00 (d, 1H), 6.92 (d, 2H), 6.52 (d, 1H), 4.66 (s, 2H), 4.28 (q, 2H), 3.48 (s, 3H), 3.41 (s, 3H), 1.30 (t, 3H).

b) A solution of the above ester (1.18 g, 3.025 mmol) in triethyl phosphite (5 mL) was refluxed for 7 hours. The resulting mixture was cooled, the precipitate collected by filtration and washed with ethyl ether to yield [4-(1,3-diethyl-2,4-dioxo-2,3,4,5-tetrahydro-1H-pyrrolo[3,2-d]pyrimidin-6-yl)phenoxy]acetic acid ethyl ester (0.32 g, 30%) as a white solid.

m.p.(MeOH/H₂O): 243-245° C.

δ ¹H NMR (DMSO): 12.45 (bs, 1H), 7.95 (d, 2H), 7.10 (d, 2H), 6.72 (s, 1H), 4.94 (s, 2H), 4.28 (q, 2H), 3.52 (s, 3H), 3.36 (s, 3H), 1.32 (t, 3H).

ESI/MS (m/e, %): 357 (M⁺, 80), 270 (100).

Preparation 4

### [4-(1,3-Dimethyl-2,4-dioxo-2,3,4,5 tetrahydro-1H-pyrrolo[3,2-d]-pyrimidin-6-yl)phenoxy]acetic Acid

Obtained as a white solid (44% overall) from 1,3,6-trimethyl-5-nitro-1H-pyrimidine-2,4-dione and (4-formylphenoxy)acetic acid following the procedure described in Preparation 2.

m.p.(MeOH/H₂O): 261-263° C.

δ ¹H NMR (DMSO): 12.89 (bs, 1H), 12.19 (s, 1H), 7.76 (d, 2H), 6.89 (d, 2H), 6.54 (d, 1H), 4.65 (s, 2H), 3.33 (s, 3H), 3.17 (s, 3H).

ESI/MS (m/e, %): 329 (M⁺, 5).

Preparation 5

### [4-(1,3-Diethyl-2,4-dioxo-2,3,4,5-tetrahydro-1H-pyrrolo[3,2-d]pyrimidin-6-yl)phenoxy]acetic Acid

Obtained as a white solid (41% overall) from 1,3-diethyl-6-methyl-5-nitro-1H-pyrimidine-2,4-dione and (4-formylphenoxy)acetic acid following the procedure described in Preparation 2.

δ H NMR (DMSO): 12.38 (bs, 1H), 7.82 (d, 2H), 7.01 (d, 2H), 6.62 (s, 1H), 4.78 (s, 2H), 3.98 (m, 4H), 10 (m, 6H).

Preparation 6

### [4-(1-Methyl-2,4 dioxo-3-propyl-2,3,4,5-tetrahydro-1H-pyrrolo [3,2-d]pyrimidin-6-yl)phenoxy]acetic Acid

Obtained as a white solid (60% overall) from 1,6-dimethyl-5-nitro-3-propyl-1H-pyrimidine-2,4-dione and (4-formylphenoxy)acetic acid following the procedure described in Preparation 2.

m.p.: 300-301° C.

δ ¹H NR (DMSO): 13.5 (bs, 1H), 12.2 (bs, 1H), 7.9 (d, 2H), 7.1 (d, 2H), 6.8 (s, 2H), 4.8 (s, 2H), 3.9 (t, 2H), 3.4 (s, 3H), 1.6 (m, 2H), 0.9 (t, 3H).

ESI/MS (m/e, %): 357 [(M+1)⁺, 91].

Preparation 7

### [4-(3-Methyl-2,4-dioxo-1-propyl-2,3,4,5-tetrahydro-1H-pyrrolo[3,2-d]pyrimidin-6-yl)-phenoxy]acetic Acid

Obtained as a yellow solid (48% overall) from 3,6-dimethyl-5-nitro-1-propyl-1H-pyrimidine-2,4-dione and (4-formylphenoxy)acetic acid following the procedure described in Preparation 2.

δ ¹H NM (DMSO): 13.0 (bs, 1H), 12.2 (bs, 1H), 7.9 (d, 2H), 7.0 (d, 2H), 6.7 (s, 2H), 4.7 (s, 2H), 3.9 (t, 2H), 3.3 (s, 3H), 1.7 (m, 2H), 0.9 (t, 3H).

Preparation 8

### {4-[1-(3-Methoxypropyl)-3-methyl-2,4-dioxo-2,3,4,5-tetrahydro-1H-pyrrolo[3,2-d]pyrimidin-6-yl]phenoxy}acetic Acid Ethyl Ester

Obtained as white solid (17% overall) from 1-(3-methoxypropyl)-3,6-dimethyl-5-nitro-H-pyrimidine-2,4-dione and (4-formylphenoxy)acetic acid ethyl ester following the procedure described in Preparation 3.

m.p.(MeOH/H₂O): 177-179° C.

δ ¹H NMR (CDCl₃): 11.7 (s, 1H), 7.85 (d, 2H), 6.95 (d, 2H), 6.46 (d, 1H), 4.67 (s, 2H), 4.30 (q, 2H), 4.07 (t, 2H), 3.48 (s, 3H), 3.43 (m, 2H), 3.34 (s, 1.32 (t, 3H).

ESI/MS (m/e, %): 415 (M⁺, 65).

Preparation 9

### [4-(3-Isobutyl-1-methyl-2,4-dioxo-2,3,4, tetrahydro-1H-pyrrolo[3,2-d]pyrimidin-6-yl)phenoxy]acetic Acid

Obtained as a white solid (50% overall) from 3-isobutyl-1,6-dimethyl-5-nitro-1H-pyrimidine-2,4-dione and (4-formylphenoxy)acetic acid following the procedure described in Preparation 2.

δ ¹H NMR (DMSO): 13.00 (bs, 1H), 12.45 (bs, 1H), 7.95 (m, 2H), 6.90 (m, 2H), 6.72 (s, 1H), 4.74 (s, 2H), 3.72 (d, 2H), 3.26 (s, 3H), 2.10 (m, 1H), 0.90 (d, 6H).

Preparation 10

### [4-(2,4-Dioxo-1-propyl-2,3,4,5-tetrahydro-1H-pyrrolo[3,2-d]pyrimidin-6-yl)phenoxy]acetic Acid

Obtained as a yellow solid (45% overall) from 6-methyl-5-nitro-1-propyl-1H-pyrimidine-2,4-dione and (4-formylphenoxy)acetic acid following the procedure described in Preparation 2.

m.p.: 306-307° C.

δ ¹H NMR (DMSO): 11.99 (bs, 1H), 10.57 (s, 1H), 7.62 (d, 2H), 6.75 (d, 2H), 6.40 (s, 1H), 4.51 (s, 2H), 3.57 (t, 2H), 1.44 (m, 2H), 0.68 (t, 3H).

Preparation 11

### {4-[1,3-Bis(2-methoxyethyl)-2,4-dioxo-2,3,4,5-tetrahydro-1H-pyrrolo[3,2-d]pyrimidin-6-yl]phenoxy}acetic Acid

Obtained as a white solid (30% overall) from 5-amino-1,3-bis(2-methoxyethyl)-6-methyl-1H-pyrimidine-2,4-dione and (4-formylphenoxy)acetic acid following the procedure described in Preparation 2.

δ ¹H NMR (DMSO): 13.10 (bs, 1H), 12.25 (bs, 1H), 7.82 (d, 2H), 7.05 (d, 2H), 6.63 (s, 1H), 4.78 (s, 2H), 4.05 (m, 4H), 3.58 (m, 4H), 3.38 (s, 3H), 3.24 (s, 3H).

Preparation 12

### {4-(1,3-Bis(cyclopropylmethyl)-2,4-dioxo-2,3,4,5-tetrahydro-1H-pyrrolo[3,2-d]pyrimidin-6-yl]phenoxy}acetic Acid

Obtained as a white solid (45% overall) from 5-amino-1,3-bis(cyclopropylmethyl)-6-methyl-1H-pyrimidine-2,4-dione and (4-formylphenoxy)acetic acid following the procedure described in Preparation 2.

δ ¹H NMR (DMSO): 13.10 (bs, 1H), 12.28 (bs, 1H), 7.88 (d; 2H); 7.02 (d, 2H), 6.72 (s, 1H), 4.76 (s, 2H), 3.81 (m, 4H), 1.25 (m, 2H), 0.38 (in, 8H).

Preparation 13

### [4-7-Chloro-1,3-dimethyl-2,4-dioxo-2,3,4,5-tetrahydro-1H-pyrrolo[3,2-d]pyrimidin-6-yl)phenoxy]acetic Acid

To a solution of the title compound of Preparation 4 (0.5 g, 1.52 mmol) in glacial acetic acid (3 mL) was slowly added sulfuryl chloride (0.13 mL) and the mixture was stirred at room temperature for 4 hours. The reaction mixture was carefully poured into stirred ice-water and the aqueous suspension was partitioned between dichloromethane and brine, then the organic phase was separated, washed with water, dried (MgSO₄) and evaporated under reduced pressure to yield the title product (500 mg, 90%) as an off white solid.

δ ¹H NMR (DMSO): 12.7 (s, 1H), 7.6 (d, 2H), 7.0 (d, 2H), 4.8 (s, 2H), 3.7 (s, 3H), 3.3 (s, 3H).

Preparation 14

### [4-(7-Bromo-2,4-dioxo-1,3-dipropyl-2,3,4,5-tetrahydro-1-H-pyrrolo[3,2-d]pyrimidin-6-yl)phenoxy]acetic Acid

To a solution of the title compound of Preparation 2 (1 g, 2.59 mmol) in glacial acetic acid (22 mL) was slowly added bromine (0.187 mL, 3.63 mmol) and the mixture was stirred at room temperature for 1 hour. Then the reaction mixture was poured into ice-water and partitioned between dichloromethane and brine, the organic phase was separated, dried (MgSO₄) and evaporated under reduced pressure to yield the title product (0.88 g, 73%) as an orange solid.

δ ¹H NMR (DMSO): 12.7 (s, 1H), 7.5 (d, 2H), 6.9 (d, 2H), 4.7 (s, 2H), 4.1 (t, 2H), 3.8 (t, 2H), 1.5 (m, 4H), 0.86 (dt, 6H).

Preparation 15

### [4-(7-Chloro-2,4-dioxo-1,3-dipropyl-2,3,4,5-tetrahydro-1H-pyrrolo[3,2-d]pyrimidin-yl)phenoxy]acetic Acid

Obtained as a yellow solid (89%) from the title compound of Preparation 2 following the procedure described in Preparation 13.

δ ¹H NMR (DMSO): 12.7 (s, 1H), 7.6 (d, 2H), 7.0 (d, 2H), 4.7 (s, 2H), 4.1 (t, 2H), 3.9 (t, 2H), 1.6 (m, 4H), 0.9 (dt, 6H).

Preparation 16

### [4-(1,3-Dimethyl-2,4-dioxo-2,3,4,5-tetrahydro-1H-pyrrolo[3,2-d]pyrimidin-6-yl)-3-methoxyphenoxy]acetic Acid

a) Following the same procedure as in Preparation 3, from 1,3,6-trimethyl-5-nitro 1H-pyrimidine-2,4-dione and (4-formyl-3-methoxyphenoxy)acetic acid ethyl ester, [4-(1,3-dimethyl-2,4-dioxo-2,3,4,5-tetrahydro-1H-pyrrolo[3,2-d]pyrimidin-6-yl)-3-methoxyphenoxy]acetic acid ethyl ester was obtained (50% overall) as a yellow solid.

m.p.(EtOH/H₂O): 234-236° C.

δ ¹H NMR (DMSO): 11.75 (bs, 1H), 7.66 (d, 1H), 6.65 (d, 1H), 6.54 (dd, 1H), 6.48 (s, 1H), 4.81 (s, 2H), 4.14 (q, 2H), 3.83 (s, 3H), 3.36 (s, 3H), 3.20 (s, 3H), 1.17 (t, 3H).

ESI/MS (m/e, %): 387 (M⁺, 100).
b) A stirred mixture of the above compound (1.43 g, 3.7 mmol) and 10% NaOH (37 mL) in ethanol (37 mL) was heated to reflux temperature for 1 hour. The resulting mixture was concentrated under reduced pressure and the residue was treated with 10% HCl. The precipitate was collected by filtration and washed with EtOH to yield the title product (1.3 g, 99%) as a white solid.

m.p.(MeOH/H₂O): >260° C. (dec.).

δ ¹H NM (DMSO): 1.184 (bs, 1H), 7.72 (d, 1H), 6.71 (s, 1H), 6.54 (m, 2H), 4.77 (s, 2H), 3.89 (s, 3H), 3.43 (s, 3H), 3.27 (s, 3H).

Preparation 17

### [4-(1,3-Dimethyl-2,4-dioxo-2,3,4,5-tetrahydro-1H-pyrrolo [3,2-d]pyrimidin-6-yl)-2-methoxyphenoxy]acetic Acid

Obtained as a white solid (25% overall) from 1,3,6-trimethyl-5-nitro-1H-pyrimidine-2,4-dione and (4-formyl-2-methoxyphenoxy)acetic acid ethyl ester following the procedure described in Preparation 16.

m.p.(MeOH/H₂O): >300° C. (dec.).

δ ¹H NMR (DMSO): 12.3 (bs, 1H), 7.60 (s, 1H), 7.42 (d, 1H), 6.91 (d, 1H), 6.68 (s, 1H), 4.73 (s, 2H), 3.88 (s, 3H), 3.43 (s, 3H), 3.27 (s, 3H).

Preparation 18

### 2-[4-(2,4-Dioxo-1,3-dipropyl-2,3,4,5 tetrahydro-1H-pyrrolo[3,2-d]pyrimidin-6-yl)phenoxy]propionic Acid

Obtained as a yellow solid (41% overall) from 1,3-dipropyl-6-methyl-5-nitro-1H-pyrimidine-2,4-dione and 2-(4-formylphenoxy)propionic acid following the procedure described in Preparation 2.

δ ¹H NMR (DMSO): 11.5 (s, 1H), 7.5 (d, 2H), 7.0 (d, 2H), 6.1 (s, 1H), 4.9 (q, 1H), 4.0 (t, 2H), 3.9 (t, 2H), 1.8 (d, 3H), 1.7 (m, 4H), 0.9 (t, 6H).

Preparation 19

### 2-[4-(1,3-Dimethyl-2,4-dioxo-2,3,4,5-tetrahydro-1H-pyrrolo[3,2-d]pyrimidin-6-yl)phenoxy]propionic Acid

Obtained as a yellow solid (53% overall) from 1,3,6-trimethyl-5-nitro-H-pyrimidine-2,4-dione and 2-(4-formylphenoxy)propionic acid following the procedure described in Preparation 2.

δ ¹H NMR (DMSO): 12.2 (s, 1H), 7.8 (d, 2H), 6.9 (d, 2H), 3.9 (m, 1H), 3.4 (s, 3H), 3.2 (s, 3H), 0.9 (dt, 3H).

Preparation 20

### 2-[4-(2,4-Dioxo-1,3-dipropyl-2,3,4, tetrahydro-1H-pyrrolo[3,2-d]pyrimidin-6-yl)phenoxy]butyric Acid

Obtained as white solid (65% overall) from 6-methyl-5-nitro-1,3-dipropyl-1H pyrimidine-2,4-dione and 2-(4-formylphenoxy)butyric acid following the procedure described in Preparation 2.

δ ¹H NMR (CDCl₃): 11.60 (bs, 1H), 7.51 (d, 2H), 7.02 (d, 2H), 4.78 (t, 1H), 4.05 (t, 2H), 3.94 (t, 2H), 2.18 (m, 2H), 1.77 (m, 4H), 1.22 (t, 3H), 0.98 (dt, 6H).

Preparation 21

### 2-[4-(2,4-Dioxo-1,3-dipropyl-2,3,4,5-tetrahydro-1H-pyrrolo[3,2-d]pyrimidin-6-yl)phenoxy]-2-methylpropionic Acid

Obtained as white-solid (25% overall) from 6-methyl-5-nitro-1,3-dipropyl-1H pyrimidine-2,4-dione and 2-(4-formylphenoxy)₂-methylpropionic acid following the procedure described in Preparation 2.

δ ¹H NMR (CDCl₃): 11.6 (s, 1H), 7.4 (d, 2H), 7.0 (d, 2H), 6.0 (s, 1H), 4.0 (t 2H), 1.7 (, 10H), 0.9 (t, 6H).

Preparation 22

### [4-(2,4-Dioxo-1,3-dipropyl-2,3,4,5-tetrahydro-1H-pyrrolo[3,2-d]pyrimidin-6-yl)phenoxy]phenylacetic Acid

Obtained as white solid (90% overall) from 6-methyl-5-nitro-1,3-dipropyl-1H-pyrimidine-2,4-dione and (4-formylphenoxy)phenylacetic acid following the procedure described in Preparation 2.

δ ¹H NMR (CDCl₃): 11.5 (s, 1H), 7.7 (d, 2H), 7.5 (d, 2H), 7.1 (d, 2H), 6.1 (s, 1H), 5.8 (s, 1H), 3.9 (m, 4H), 1.7 (m, 4H), 0.9 (dt, 6H).

Preparation 23

### 3-[4-(2,4-Dioxo-1,3-dipropyl-2,3,4,5-tetrahydro-1H-pyrrolo[3,2-d]pyrimidin-6-yl)phenyl]propionic Acid

Obtained as white solid (22% overall) from 6-methyl-5-nitro-1,3-dipropyl-1H pyrimidine-2,4-dione and 3-(4-formylphenyl)propionic acid following the procedure described in Preparation 2.

δ ¹H NMR (CDCl₃): 12.3 (s, 1H), 12.1 (s, 1H), 7.8 (2H, d), 7.3 (d, 2H), 6.7 (s, 1H), 3.8 (m, 4H), 2.8 (t, 2H), 2.5 (t, 2H), 1.6 (m, 4H), 0.9 (dt, 6H).

Preparation 24

### 3-[4-(2,4-Dioxo-1,3-dipropyl-2,3,4,5-tetrahydro-1H-pyrrolo[3,2-d]pyrimidin-6-yl)phenyl]acrylic Acid

Obtained as white-solid (20% overall) from 6-methyl-5-nitro-1,3-dipropyl-1H pyrimidine-2,4-dione and 3)₄-formylphenyl)acrylic acid following the procedure described in Preparation 2.

δ ¹H NMR (DMSO): 12.3 (s, 1H), 7.9 (d, 2H), 7.7 (d, 2H), 7.5 (d, 1H), 6.8 (s, 1H), 6.5 (d, 1H), 3.8 (m, 4H), 1.5 (m, 4H), 0.8 (dt, 6H).

Preparation 25

### 4-[4-(2,4-Dioxo-1,3]-dipropyl-2,3,4,5-tetrahydro-1H-pyrrolo[3,2-d]-pyrimidin-6-yl)phenoxy]butyric Acid

Obtained as white solid (45% overall) from 6-methyl-5-nitro-1,3-dipropyl-1H pyrimidine-2,4-dione and 4-(4-formylphenoxy)butyric acid following the procedure described in Preparation 2.

δ ¹H NMR (CDCl₃): 11.7 (s, 1H), 7.7 (d, 2H), 6.9 (d, 2H), 6.1 (s, 1H), 4.2 (bs, 2H), 3.9 (m, 4H), 2.1 (bs, 2H), 1.7 (m, 4H), 0.9 (m, 6H).

Preparation 26

### 4-(2,4-Dioxo-1,3-dipropyl-2,3,4,5 tetrahydro-1H-pyrrolo [3,2-d]pyrimidin-6-yl)benzoic Acid

Obtained as yellow solid (36% overall) from 6-methyl-5-nitro-1,3-dipropyl-1H pyrimidine-2,4 dione and 4-formylbenzoic acid following the procedure described in Preparation 2.

δ ¹H NMR (DMSO): 13.0 (bs, 1H), 12.6 (s, 1H), 8.0 (dd, 4H), 6.9 (s, 1H), 3.9 (m, 4H) 1.6 (m, 4H), 0.9 (dt, 6H).

Preparation 27

### [4-(2,4-Dioxo-3-propyl-2,3,4,5-tetrahydro-1H-pyrrolo[3,2-d]pyrimidin-6-yl)phenoxy]acetic Acid

Obtained as a yellow solid (6% overall) from 6-methyl-5-nitro-3-propyl-1H-pyrimidine-2,4-dione and (4-formylphenoxy)acetic acid following the same procedure described in Preparation 2.

δ ¹H NM (DMSO): 12.9 (s, 1H), 11.9 (s, 1H), 11.0 (s, 1H), 7.8 (d, 2H), 6.9 (d, 2H), 6.2 (d, 1H), 4.7 (s, 2H), 3.8 (t, 2H), 1.6 (m, 2H), 0.9 (t, 3H).

Preparation 28

### {4-[3-Methyl-1-(3-morpholin-4-ylpropyl)-2,4-dioxo-2,3,4,5-tetrahydro-1H-pyrrolo[3,2-d]pyrimidin-6-yl]phenoxy}acetic Acid Hydrochloride

a) A solution of 3,6-dimethyl-1-(3-morpholin-4-ylpropyl)₅-nitro-1H-pyrimidine-2,4-dione (0.50 g, 1.60 mmol), (4-formylphenoxy)acetic acid (0.31 g, 1.76 mmol) and piperidine (79 μL, 0.80 mmol) in ethanol (8 mL) with 3A molecular sieves (0.83 g) was refluxed for 3 hours. The resulting suspension was filtrated and the filtrates were evaporated under reduced pressure. The residue was suspended in water (100 mL), extracted with dichloromethane and water was evaporated under reduced pressure to yield (4 {-2-[1-methyl-3-(3-morpholin 4-ylpropyl)-5-nitro-2,6-dioxo-12,3,6 tetrahydropyridin-4-yl]vinyl}phenoxy)acetic acid (0.76 g, 100%) as a yellow solid.

b) To a stirred solution of the above compound (0.76 g, 1.60 mmol) in formic acid (15 mL) was slowly added sodium dithionite (1.64 g, 8.00 mmol) and the mixture was refluxed overnight. The solvent was evaporated under reduced pressure, the residue was redissolved in a mixture of dichloromethane and methanol and the insoluble salts were separated by filtration. The filtrates were acidified until pH 3 by adding dioxane saturated with hydrochloric acid, the solvent was evaporated under reduced pressure and the residue was triturated with a mixture of dichloromethane-diethyl ether, to yield the title compound as a dark yellow solid (0.70 g, 99%).

δ ¹H NMR (CDCl₃): 9.8 (s, 1H), 7.8 (d, 2H), 6.9 (d, 2H), 6.6 (s, 1H), 4.6 (s, 2H) 3.9 (t, 2H), 3.6 (m, 4H), 3.3 (s, 3H), 2.3 (m, 6H), 1.8 (m, 2H).

Preparation 29

### 6-(4-Hydroxymethylphenyl)-1,3-dimethyl-1,5-dihydropyrrolo[3,2-d]pyrimidine 2,4-dione

a) To a solution of 6-methyl-5-nitro-1,3-dimethyl-1H-pyrimidine-2,4-dione (1.59 g, 7.99 mmol) in dry dioxane (50 mL) was added piperidine (1.18 mL, 11.99 mmol), 4-formylbenzoic acid (1.44 g, 7.99 mmol) and 3 A molecular sieves. The mixture was stirred at 50° C. for 5 hours. The resulting solution was concentrated under vacuum and the residue was treated with ethyl acetate, washed with 10% aqueous hydrochloric acid (3×50 mL) and brine (3×50 mL), dried (Na₂SO₄) and evaporated under reduced pressure. The obtained residue was crystalized from ethanol to yield 4-[2-(1,3-dimethyl-5-nitro-2,6-dioxo-1,2,3,6-tetrahydropyrimidin-4-yl)vinyl]benzoic acid (1.89 g, 70%) as a yellow solid.

b) To a stirred solution of the above compound (0.50 g, 1.51 mmol) in formic acid (15 mL) was slowly added sodium dithionite (1.84 g, 10.56 mmol) and the mixture was refluxed for 24 hours. The resulting solution was cooled to room temperature and poured into water. The resulting precipitate was collected by filtration, washed with water and dried under vacuum to yield 4-(1,3-dimethyl-2,4-dioxo-2,3,4,5-tetrahydro-1H-pyrrolo[3,2-d]pyrimidin-6-yl)benzoic acid (0.37 e, 80%) as a white solid.

c) To a stirred solution of the above compound (0.20 g, 0.66 mmol) in dry tetrahydrofuran (3 mL) at 0° C. and under argon atmosphere, was slowly added a 1 M solution of borane in tetrahydrofuran (6.67 mL, 6.67 mmol) and the mixture was refluxed for 24 hours. The resulting solution was cooled to room temperature, methanol was slowly added and the solvent was evaporated under reduced pressure. The residue was suspended in ethyl acetate (100 mL), washed with 10% aqueous sodium hydroxide (2×10 mL) and water (10 mL). The organic layer was dried (Na₂SO₄) and evaporated under reduced pressure. The obtained residue was crystalized from a mixture of dietyl ether and methanol to yield the title compound (0.080 g, 40%) as a white solid.

δ ¹H NMR (CDCl₃): 12.3 (bs, 1H), 7.8 (d, 2H), 7.3 (d, 2H), 6.7 (s, 1H), 5.12 (m, 1H), 4.5 (d, 2H), 3.4 (s, 3H), 3.2 (s, 3H).

Preparation 30

### 6-(4-Hydroxymethylphenyl)-1,3-dipropyl-1,5-dihydropyrrolo[3,2-d]pyrimidine-2,4-dione

Obtained as a white solid (65% overall) from the title compound of Preparation 26 following the procedure described in Preparation 29c.

δ ¹H NMR (CDCl₃): 12.2 (bs, 1H), 7.7 (d, 2H), 7.2 (d, 2H), 6;7 (d, 1H), 5.12 (m, 1H), 4.4 (d, 2H), 3.7 (in, 411), 1.4-1.55 (m, 4H), 0.65-0.8 (m, 6H).

TABLE 3

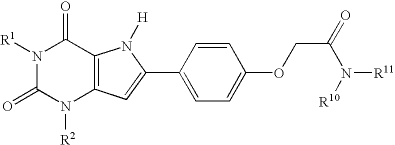

| Example | | | |
|---|---|---|---|
| No | R¹ | R² | NR¹⁰R¹¹ |
| 1 | nPr | nPr | 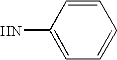 |
| 2 | nPr | nPr | 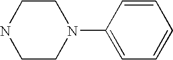 |
| 3 | nPr | nPr | 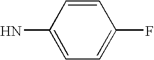 |
| 4 | nPr | nPr | 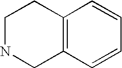 |
| 5 | nPr | nPr | 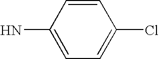 |
| 6 | nPr | nPr | 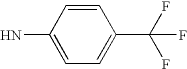 |
| 7 | nPr | nPr | 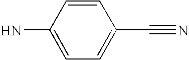 |
| 8 | nPr | nPr | 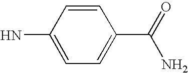 |
| 9 | nPr | nPr | 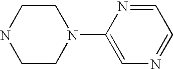 |
| 10 | nPr | nPr | 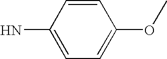 |
| 11 | nPr | nPr | 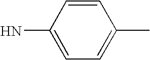 |
| 12 | nPr | nPr | 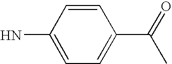 |
| 13 | nPr | nPr | 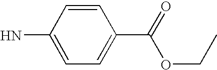 |
| 14 | nPr | nPr | 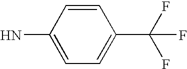 |
| 15 | nPr | nPr | 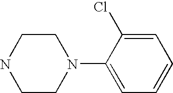 |
| 16 | nPr | nPr | 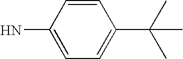 |
| 17 | nPr | nPr | 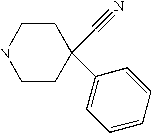 |
| 18 | nPr | nPr | 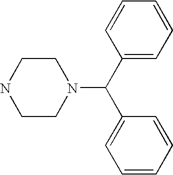 |
| 19 | nPr | nPr | 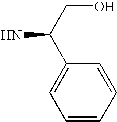 |
| 20 | nPr | nPr | 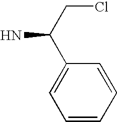 |
| 21 | nPr | nPr | 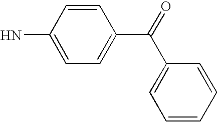 |
| 22 | nPr | nPr | 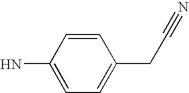 |
| 23 | nPr | nPr | 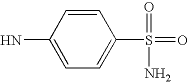 |
| 24 | nPr | nPr | 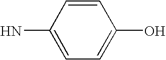 |
| 25 | nPr | nPr | 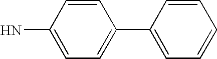 |
| 26 | nPr | nPr | 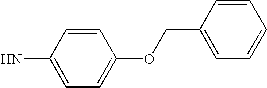 |
| 27 | nPr | nPr | 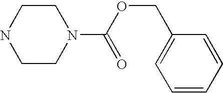 |
| 28 | nPr | nPr | 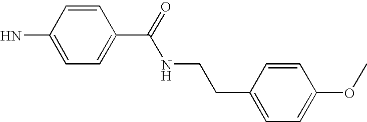 |
| 29 | nPr | nPr | 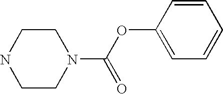 |
| 30 | nPr | nPr | 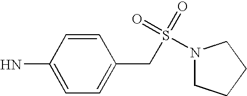 |
| 31 | nPr | nPr | 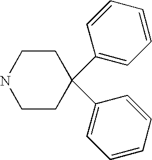 |
| 32 | nPr | nPr | 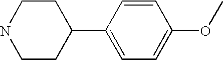 |
| 33 | nPr | nPr | 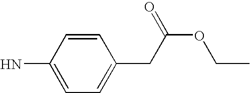 |
| 34 | nPr | nPr | 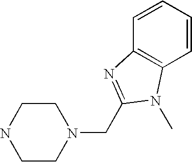 |
| 35 | nPr | nPr | 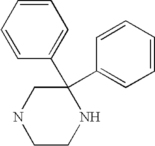 |
| 36 | nPr | nPr | 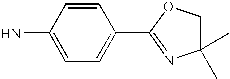 |
| 37 | nPr | nPr | 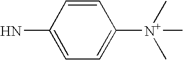 |
| 38 | nPr | nPr | 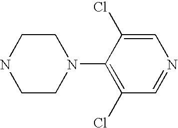 |
| 39 | nPr | nPr | 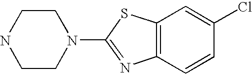 |
| 40 | nPr | nPr | 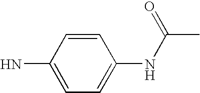 |
| 41 | nPr | nPr | 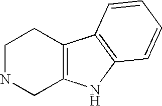 |
| 42 | nPr | nPr | 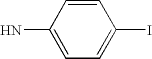 |
| 43 | nPr | nPr | 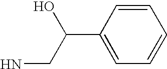 |
| 44 | nPr | nPr | 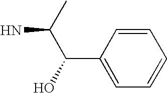 |
| 45 | nPr | nPr | 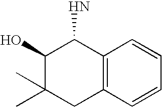 |
| 46 | nPr | nPr | 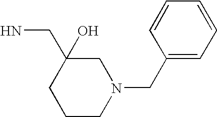 |
| 47 | nPr | nPr | 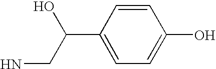 |
| 48 | nPr | nPr | 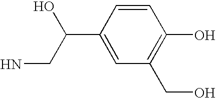 |
| 49 | nPr | nPr | 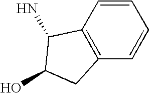 |
| 50 | nPr | nPr | 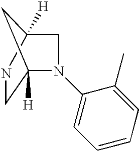 |
| 51 | nPr | nPr | 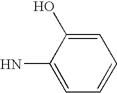 |
| 52 | nPr | nPr | 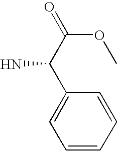 |
| 53 | nPr | nPr | 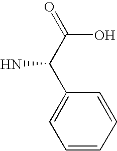 |
| 54 | nPr | nPr | 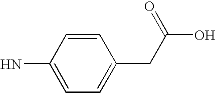 |
| 55 | Me | Me | 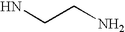 |
| 56 | Me | Me | 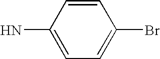 |
| 57 | Me | Me | 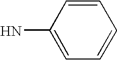 |
| 58 | Me | Me | 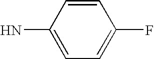 |
| 59 | Me | Me | 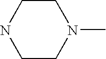 |
| 60 | Me | Me | 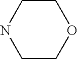 |
| 61 | Me | Me | 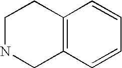 |
| 62 | Me | Me | 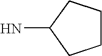 |
| 63 | Me | Me | 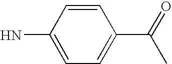 |
| 64 | Me | Me | 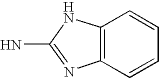 |
| 65 | Me | Me | 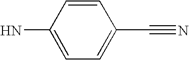 |
| 66 | Me | Me | 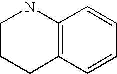 |
| 67 | Me | Me | 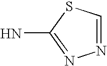 |
| 68 | Me | Me | 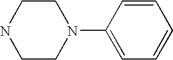 |
| 69 | Me | Me | 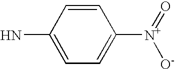 |
| 70 | Me | Me | 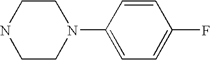 |
| 71 | Me | Me | 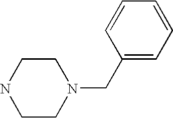 |
| 72 | Me | Me | 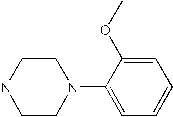 |
| 73 | Me | Me | 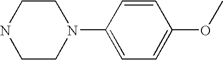 |
| 74 | Me | Me | 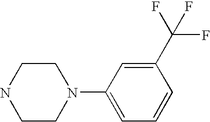 |
| 75 | Me | Me | 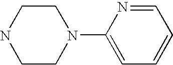 |
| 76 | Me | Me | 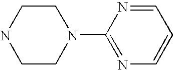 |
| 77 | Me | Me | 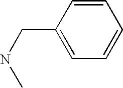 |
| 78 | Me | Me | 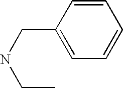 |
| 79 | Me | Me | 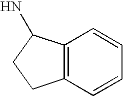 |
| 80 | Me | Me | 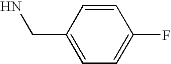 |
| 81 | Me | Me | 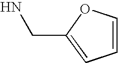 |
| 82 | Me | Me | 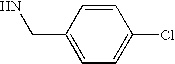 |
| 83 | Me | Me | 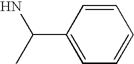 |
| 84 | Me | Me | 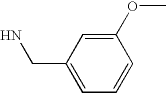 |
| 85 | Me | Me | 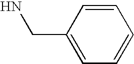 |
| 86 | Me | Me | 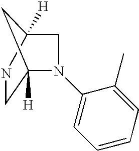 |
| 87 | Et | Et | 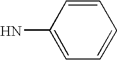 |
| 88 | Et | Et | 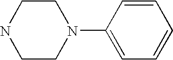 |
| 89 | Et | Et | 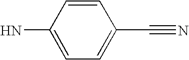 |
| 90 | nPr | Me | 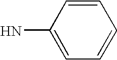 |
| 91 | nPr | Me | 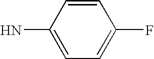 |
| 92 | nPr | Me | 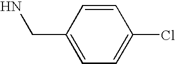 |
| 93 | nPr | Me | 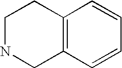 |
| 94 | nPr | Me | 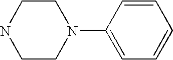 |
| 95 | nPr | Me | 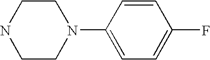 |
| 96 | nPr | Me | 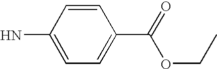 |
| 97 | nPr | Me | 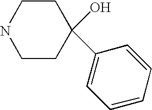 |
| 98 | nPr | Me | 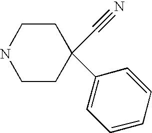 |
| 99 | nPr | Me | 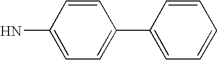 |
| 100 | nPr | Me | 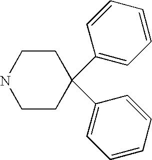 |
| 101 | nPr | Me | 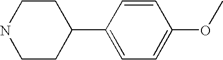 |
| 102 | nPr | Me | 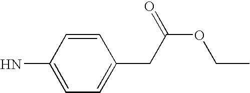 |
| 103 | nPr | Me | 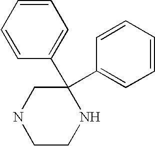 |
| 104 | nPr | Me | 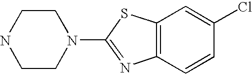 |
| 105 | nPr | Me | 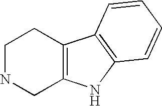 |
| 106 | nPr | Me | 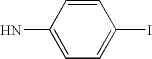 |
| 107 | nPr | Me | 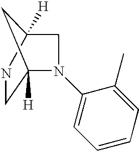 |
| 108 | Me | nPr | 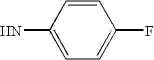 |
| 109 | Me | nPr | 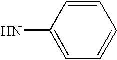 |
| 110 | Me | nPr | 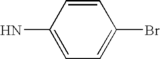 |
| 111 | Me | nPr | 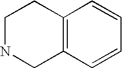 |
| 112 | Me | nPr | 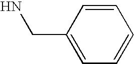 |
| 113 | Me | nPr | 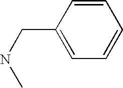 |
| 114 | Me | nPr | 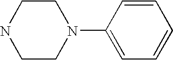 |
| 115 | Me | nPr | 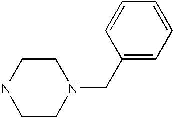 |
| 116 | Me | nPr | 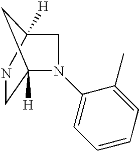 |
| 117 | Me | MeOPro | 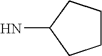 |
| 118 | Me | MeOPro | 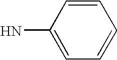 |
| 119 | i-Bu | Me | 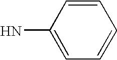 |
| 120 | i-Bu | Me | 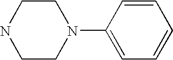 |
| 121 | H | nPr | 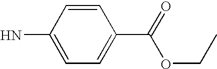 |
| 122 | H | nPr | 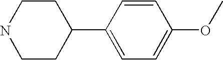 |
| 123 | H | nPr | 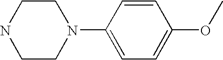 |
| 124 | H | nPr | 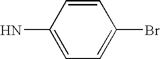 |
| 125 | H | nPr | 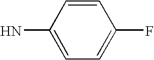 |
| 126 | MeOEt | MeOEt | 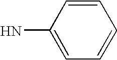 |
| 127 | MeOEt | MeOEt | 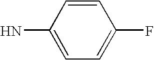 |
| 128 | MeOEt | MeOEt | 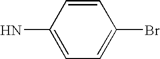 |
| 129 | MeOEt | MeOEt | 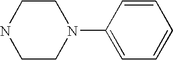 |
| 130 | MeOEt | MeOEt | 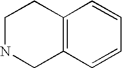 |
| 131 | 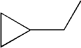 | 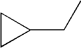 | 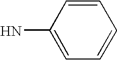 |
| 132 | 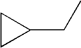 | 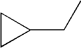 | 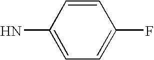 |
| 133 | 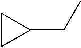 | 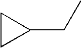 | 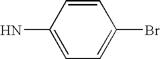 |
| 134 | 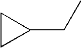 | 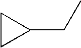 | 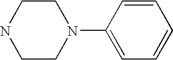 |
| 135 | 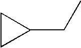 | 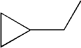 | 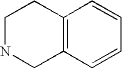 |

TABLE 4

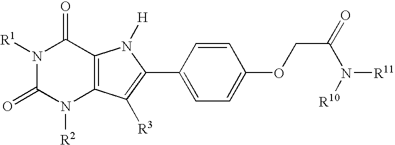

| Example | | | | |
|---|---|---|---|---|
| No | R¹ | R² | R³ | NR¹⁰R¹¹ |
| 136 | Me | Me | Cl | 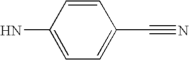 |
| 137 | nPr | nPr | Br | 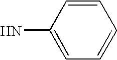 |
| 138 | nPr | nPr | Br | 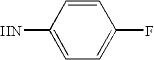 |
| 139 | nPr | nPr | Cl | 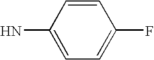 |
| 140 | nPr | nPr | Cl | 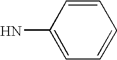 |
| 141 | nPr | nPr | Cl | 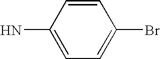 |
| 142 | nPr | nPr | Cl | 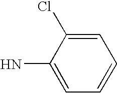 |
| 143 | nPr | nPr | Cl | 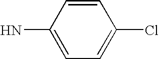 |
| 144 | nPr | nPr | Cl | 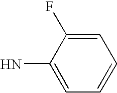 |
| 145 | nPr | nPr | Cl | 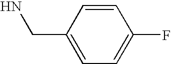 |
| 146 | nPr | nPr | Cl | 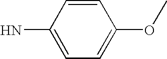 |
| 147 | nPr | nPr | Cl | 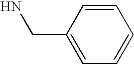 |
| 148 | nPr | nPr | Cl | 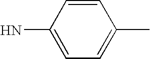 |
| 149 | nPr | nPr | Cl | 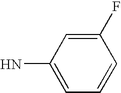 |

TABLE 5

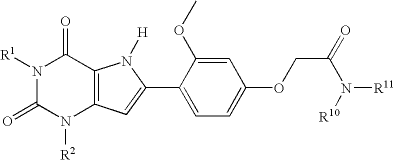

| Example | | | |
|---|---|---|---|
| No | R¹ | R² | NR¹⁰R¹¹ |
| 150 | Me | Me | 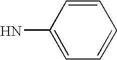 |
| 151 | Me | Me | 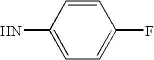 |
| 152 | Me | Me | 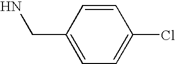 |
| 153 | Me | Me | 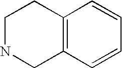 |
| 154 | Me | Me | 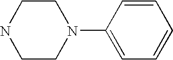 |
| 155 | Me | Me | 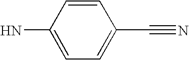 |
| 156 | Me | Me | 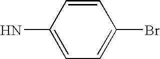 |
| 157 | Me | Me | 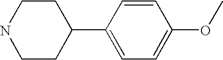 |
| 158 | Me | Me | 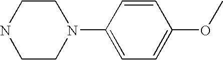 |

TABLE 6

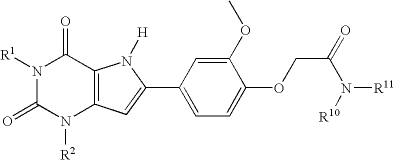

| Example | | | |
|---|---|---|---|
| No | R¹ | R² | NR¹⁰R¹¹ |
| 159 | Me | Me | 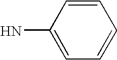 |
| 160 | Me | Me | 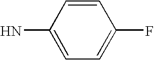 |
| 161 | Me | Me | 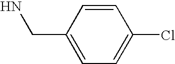 |
| 162 | Me | Me | 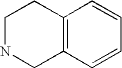 |
| 163 | Me | Me | 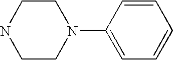 |
| 164 | Me | Me | 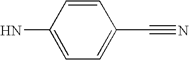 |
| 165 | Me | Me | 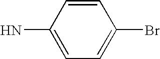 |
| 166 | Me | Me | 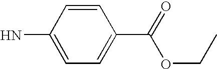 |
| 167 | Me | Me | 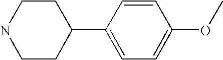 |
| 168 | Me | Me | 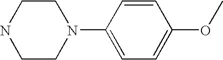 |

TABLE 7

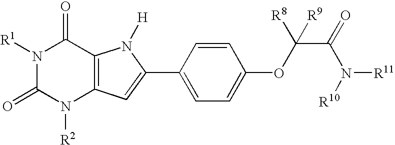

| Example | | | | | |
|---|---|---|---|---|---|
| No | R¹ | R² | R⁸ | R⁹ | NR¹⁰R¹¹ |
| 169 | nPr | nPr | H | Me | 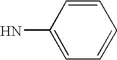 |
| 170 | nPr | nPr | H | Me | 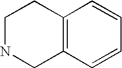 |
| 171 | nPr | nPr | H | Me | 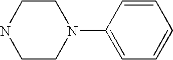 |
| 172 | nPr | nPr | H | Me | 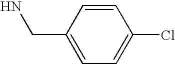 |
| 173 | nPr | nPr | H | Me | 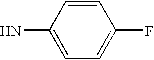 |
| 174 | nPr | nPr | H | Me | 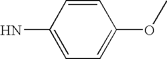 |
| 175 | Me | Me | H | Me | 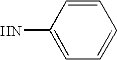 |
| 176 | Me | Me | H | Me | 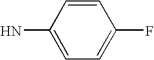 |
| 177 | Me | Me | H | Me | 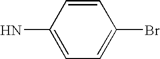 |
| 178 | Me | Me | H | Me | 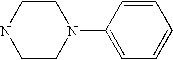 |
| 179 | Me | Me | H | Me | 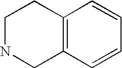 |
| 180 | nPr | nPr | H | Et | 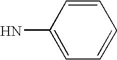 |
| 181 | nPr | nPr | H | Et | 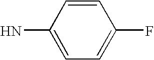 |
| 182 | nPr | nPr | H | Et | 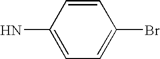 |
| 183 | nPr | nPr | H | Et | 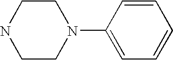 |
| 184 | nPr | nPr | H | Et | 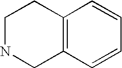 |
| 185 | nPr | nPr | Me | Me | 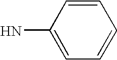 |
| 186 | nPr | nPr | Me | Me | 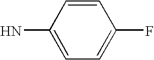 |
| 187 | nPr | nPr | Me | Me | 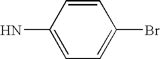 |
| 188 | nPr | nPr | Me | Me | 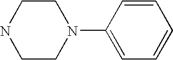 |
| 189 | nPr | nPr | Me | Me | 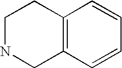 |
| 190 | nPr | nPr | H | Phe | 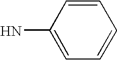 |
| 191 | nPr | nPr | H | Phe | 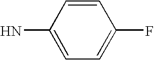 |
| 192 | nPr | nPr | H | Phe | 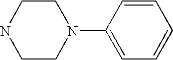 |

TABLE 8

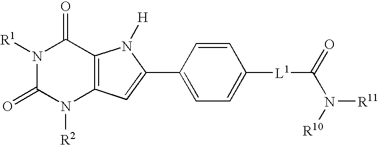

| Example | | | | |
|---|---|---|---|---|
| No | R¹ | R² | L₁ | NR¹⁰R¹¹ |
| 193 | nPr | nPr | —CH₂CH₂— | 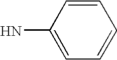 |
| 194 | nPr | nPr | —CH₂CH₂— | 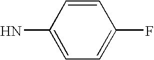 |
| 195 | nPr | nPr | —CH₂CH₂— | 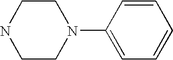 |
| 196 | nPr | nPr | —CH₂CH₂— | 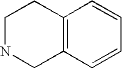 |
| 197 | nPr | nPr | —CH═CH— | 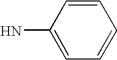 |
| 198 | nPr | nPr | —CH═CH— | 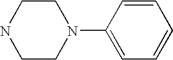 |
| 199 | nPr | nPr | —CH═CH— | 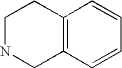 |
| 200 | nPr | nPr | —O(CH₂)₃— | 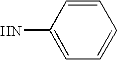 |
| 201 | nPr | nPr | —O(CH₂)₃— | 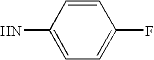 |
| 202 | nRr | nPr | —O(CH₂)₃— | 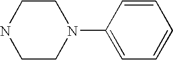 |
| 203 | nPr | nPr | —O(CH₂)₃— | 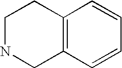 |
| 204 | nPr | nPr | —O(CH₂)₃— | 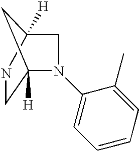 |

TABLE 9

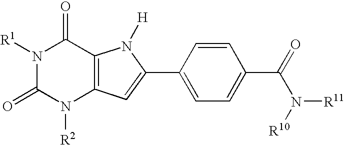

| Example | | | |
|---|---|---|---|
| No | R¹ | R² | NR¹⁰R¹¹ |
| 205 | nPr | nPr | 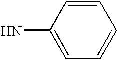 |
| 206 | nPr | nPr | 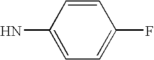 |
| 207 | nPr | nPr | 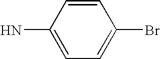 |
| 208 | nPr | nPr | 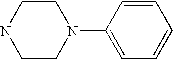 |
| 209 | nPr | nPr | 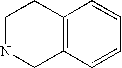 |

TABLE 10

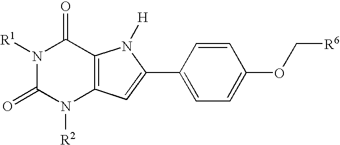

| Example | | | |
|---|---|---|---|
| No | R¹ | R² | R⁶ |
| 210 | nPr | nPr | 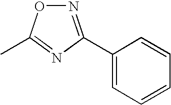 |
| 211 | nPr | nPr | 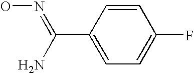 |
| 212 | nPr | nPr | 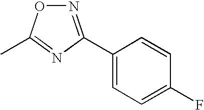 |
| 213 | nPr | nPr | 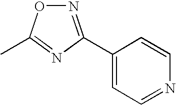 |
| 214 | nPr | nPr | 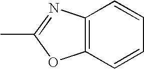 |
| 215 | nPr | nPr | 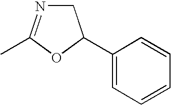 |
| 216 | nPr | nPr | 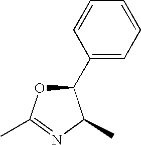 |
| 217 | nPr | nPr | 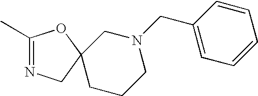 |
| 218 | nPr | nPr | 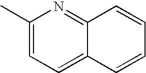 |

TABLE 11

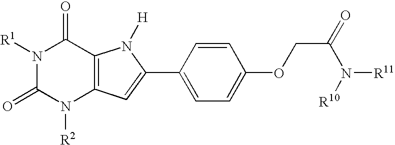

| Example | | | |
|---|---|---|---|
| No | R¹ | R² | NR¹⁰R¹¹ |
| 219 | NPr | nPr | 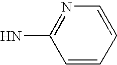 |
| 220 | nPr | nPr | 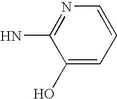 |
| 221 | nPr | nPr | 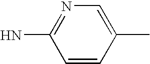 |
| 222 | nPr | nPr | 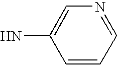 |
| 223 | nPr | nPr | 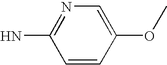 |
| 224 | nPr | nPr | 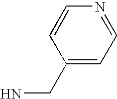 |
| 225 | nPr | nPr | 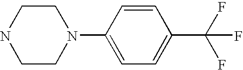 |
| 226 | nPr | nPr | 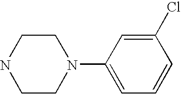 |
| 227 | nPr | nPr | 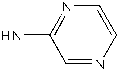 |
| 228 | nPr | nPr | 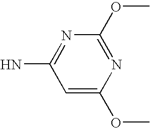 |
| 229 | nPr | nPr | 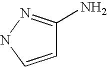 |
| 230 | Me | Me | 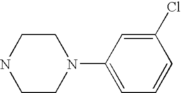 |
| 231 | Me | Me | 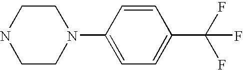 |
| 232 | Me | Me | 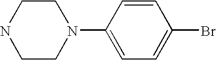 |
| 233 | Me | Me | 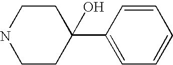 |
| 234 | Me | Me | 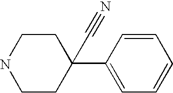 |
| 235 | Me | Me | 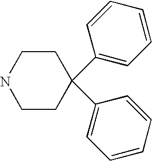 |
| 236 | Me | Me | 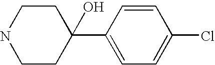 |
| 237 | Me | Me | 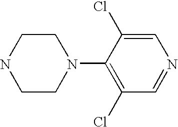 |
| 238 | Me | Me | 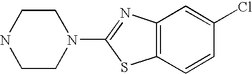 |
| 239 | Me | Me | 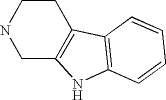 |
| 240 | Me | Me | 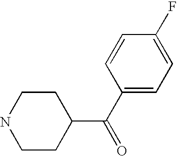 |
| 241 | Me | Me | 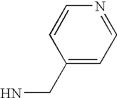 |
| 242 | Me | Me | 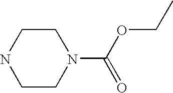 |
| 243 | nPro | Me | 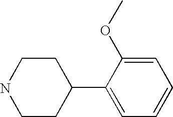 |
| 244 | nPro | Me | 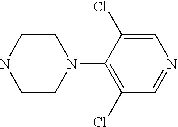 |
| 245 | nPro | Me | 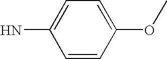 |
| 246 | nPro | Me | 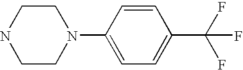 |
| 247 | nPro | H | 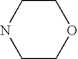 |
| 248 | nPro | H | 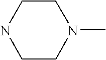 |
| 249 | nPro | H | 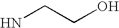 |
| 250 | nPro | H | 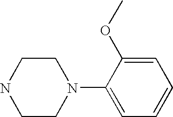 |
| 251 | nPro | H | 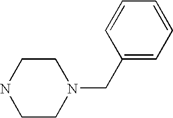 |
| 252 | nPro | H | 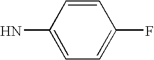 |
| 253 | nPro | H | 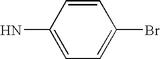 |
| 254 | H | nPro | 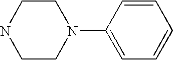 |
| 255 | Me | 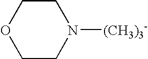 | 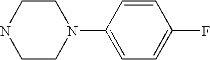 |
| 256 | Me | 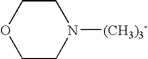 | 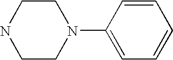 |
| 257 | Me | 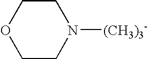 | 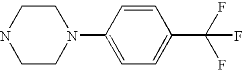 |

TABLE 12

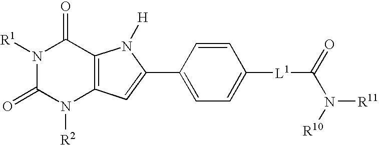

| Example | | | | |
|---|---|---|---|---|
| No | R¹ | R² | L₁ | NR¹⁰R¹¹ |
| 258 | nPr | nPr | —CH₂O— | 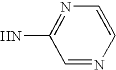 |
| 259 | nPr | nPr | —CH₂O— | 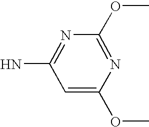 |
| 260 | nPr | nPr | —CH₂O— | 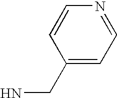 |
| 261 | nPr | nPr | —CH₂O— | 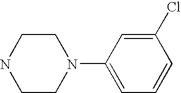 |
| 262 | nPr | nPr | —CH₂O— | 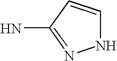 |
| 263 | nPr | nPr | —CH₂O— | 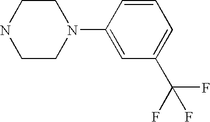 |
| 264 | nPr | nPr | —CH₂O— | 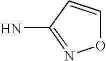 |
| 265 | Me | Me | —CH₂O— | 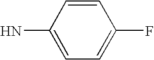 |
| 266 | Me | Me | —CH₂O— | 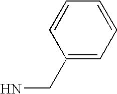 |
| 267 | Me | Me | —CH₂O— | 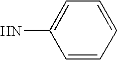 |
| 268 | Me | Me | —CH₂O— | 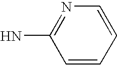 |
| 269 | Me | Me | —CH₂O— | 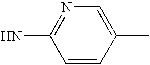 |
| 270 | Me | Me | —CH₂O— | 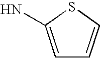 |
| 271 | Me | Me | —CH₂O— | 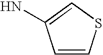 |
| 272 | Me | Me | —CH₂O— | 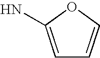 |
| 273 | Me | Me | —CH₂O— | 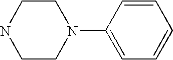 |
| 274 | Me | Me | —CH₂O— | 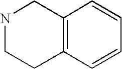 |
| 275 | nPr | nPr | —O(CH₂)₂O— | 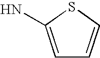 |
| 276 | nPr | nPr | —O(CH₂)₂O— | 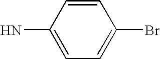 |
| 277 | nPr | nPr | —CH₂NH— | 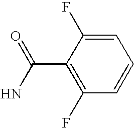 |

TABLE 13

| Example | | | | | |
|---|---|---|---|---|---|
| No | R¹ | R² | R⁸ | R⁹ | R⁶ |
| 278 | nPr | nPr | H | H | 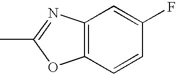 |
| 279 | nPr | nPr | H | H | 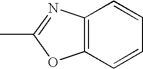 |
| 280 | Me | Me | H | H | 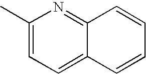 |
| 281 | Me | Me | H | H | 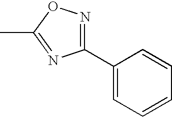 |
| 282 | nPr | Me | H | H | 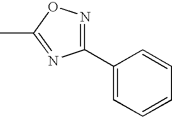 |
| 283 | nPr | Me | H | H | 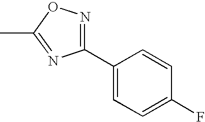 |
| 284 | nPr | Me | H | H | 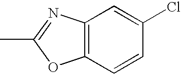 |
| 285 | nPr | H | H | H | 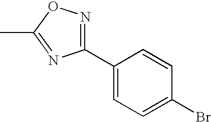 |
| 286 | Me | Me | H | Me | 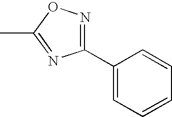 |
| 287 | Me | Me | H | Me | 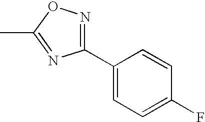 |
| 288 | Me | Me | H | Me | 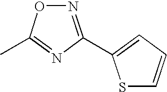 |
| 289 | Me | Me | H | Me | 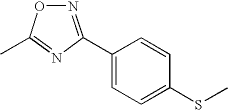 |

TABLE 14

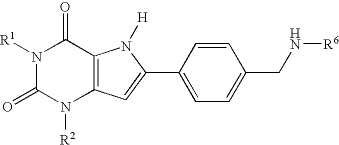

| Example | | | |
|---|---|---|---|
| No | R¹ | R² | R⁶ |
| 290 | nPr | nPr | 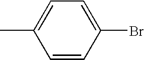 |
| 291 | nPr | nPr | 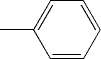 |

### Example 1

### 2-[4-(2,4-Dioxo-1,3-dipropyl-2,3,4,5 tetrahydro-1H-pyrrolo[3,2-d]pyrimidin-6-yl)phenoxy]-N-phenylacetamide

a) To a solution of the title compound of Preparation 1 (300 mg, 0.72 mmol) in anhydrous tetrahydrofuran (20 mL) under argon atmosphere was slowly added at −40° C. N-methylmorpholine (0.079 mL, 0.72 mmol) and isobutyl chloroformate (0.093 mL, 0.72 mmol). The mixture was stirred at 40° C. for 2 hours. Then aniline was added (0.066 mL, 0.72 mmol) and the mixture was stirred 15 minutes at −40° C. and 12 hours at room temperature. The resulting solution was evaporated under reduced pressure and the residue was partitioned between dichloromethane and a saturated aqueous solution of sodium bicarbonate. The organic phase was separated, washed with water and brine, dried (Na₂SO₄) and evaporated under reduced pressure. The resulting crude was purified by flash column chromatography on silica-gel (dichloromethane) to yield the intermediate amide as a yellow solid (150 mg, 42%).

m.p.(EtOH): 62-64° C.

δ ¹H NMR (CDCl₃): 8.18 (bs, 1H), 7.59 (d, 2H), 7.46 (d, 2H), 7.12 (m, 5H), 6.53 (d, 1H), 4.66 (s, 2H), 3.91 (m, 4H), 1.68 (m, 4H), 0.97 (m, 6H).

ESI/MS (m/e,%): 492 (M⁺, 46).

b) A stirred solution of the above compound (150 mg, 0.305 mmol) in triethylphosphite (2 mL) was refluxed under argon atmosphere for 5 hours. The mixture was cooled to room temperature and the resulting precipitate was collected by filtration, washed with ethyl ether and dried under vacuum to yield the title compound (65 mg, 46%) as a white solid.

m.p.(MeOH/H₂O): 257-259° C.

δ ¹H NR (DMSO): 12.20 (s, 1H), 10.10 (s, 1H), 7.87 (d, 2H), 7.63 (d, 2H), 7.32 (m, 2H), 7.07 (m, 3H), 6.65 (s, 1H), 4.75 (s, 2H), 3.85 (m, 4H), 1.61 (m, 6H).

ESI/MS (m/e,%): 460 (M⁺, 100).

### Example 2

### 6-{4-[2-Oxo-2-(4-phenylpiperazin-1-yl)ethoxy]phenyl}-1,3-dipropyl-1,5-dihydropyrrolo[3,2-d]pyrimidine-2,4-dione

Obtained as a white solid (20%) from the title compound of Preparation 1 and 1-phenylpiperazine following the procedure of example 1.

m.p.(MeOH/H₂O): 180-184° C.

δ ¹H NMR (DMSO): 12.15 (s, 1H), 7.86 (d, 2H), 7.25 (m, 2H), 7.00 (m, 4H), 6.83 (m, 1H), 6.66 (s, 1H), 4.96 (s, 2H), 3.87 (m, 4H), 3.63 (m, 4H), 3.21 (m, 2H), 3.14 (m, 2H), 2.51 (m, 4H), 0.90 (m, 6H).

ESI/MS (m/e,%): 529 (M⁺, 19).

### Example 3

### 2-[4-(2,4-Dioxo-1,3-dipropyl-2,3,4,5-tetrahydro-1H-pyrrolo[3,2-d]pyrimidin-6-yl)phenoxy]-N-(4-fluorophenyl)acetamide

Obtained as a white solid (23%) from the title compound of Preparation 1 and 4-fluoroaniline following the procedure of example 1.

m.p.(MeOH/H₂O): 256-258° C.

δ ¹H NMR (DMSO): 12.21 (s, 1H), 10.18 (s, 1H), 7.89 (m, 2H), 7.63 (m, 2H), 7.12 (m, 4H), 6.67 (s, 1H), 4.77 (s, 2H), 3.84 (m, 4H), 1.61 (m, 4H), 0.91 (m, 6H).

ESI/MS (m/e,%): 478 (M⁺, 100).

### Example 4

### 6-{4-[2-3,4-Dihydro-1H-isoquinolin-2-yl)-2-oxoethoxy]phenyl}-1,3-dipropyl-1,5-dihydropyrrolo[3,2-d]pyrimidine-2,4-dione

To mixture of the title compound of Preparation 2 (480 mg, 1.24 mmol), N-3-dimethylaminopropyl)-N′-ethyl carbodiimide hydrochloride (285 mg, 1.49 mmol), 1-hydroxybenzotriazole (201 mg, 1.49 mmol) and triethylamine (0.44 mL, 2.48 mmol) in dimethylformamide (20 mL) was added 1,2,3,4-tetrahydroisoquinoline (0.205 mL, 1.61 mmol) and the mixture was stirred at room temperature overnight. The resulting solution was evaporated under reduced pressure and the residue was partitioned between dichloromethane and a saturated aqueous solution of sodium bicarbonate. The organic phase was separated, washed with water and brine, dried (Na₂SO₄) and evaporated under reduced pressure. The resulting crude was purified by flash column chromatography on silica-gel (hexanes:ethyl acetate 1:1) to yield the title compound as a white solid (270 mg, 43%).

m.p.: 176.9-177.60° C.

δ ¹H NMR (DMSO): 12.22 (bs, 1H), 7.83 (d, 2H), 7.20 (m, 4H), 7.00 (d, 2H), 6.65 (s, 1H), 4.98 (s, 2H), 4.67 (m, 2H), 3.85 (m, 4H), 3.70 (m, 2H), 2.86 (m, 2H), 1.65 (m, 4H), 0.89 (in, 6H).

ESI/MS (m/e,%): 500 (M⁺, 82).

### Example 5

### N-(4-Chlorophenyl)-2-[4-(2,4-1,3-dipropyl-2,3,4,5-tetrahydro-1H-pyrrolo[3,2-pyrimidin-6-yl)phenoxy]acetamide

To mixture of the title compound of Preparation 2 (80 mg, 0.21 mmol), N-(3-dimethylaminopropyl)-N′-ethylcarbodiimide hydrochloride (44 mg, 0.23 mmol), 1-hydroxybenzotriazole (31 mg, 0.23 mmol) and polymer bound morpholine (280 mg, 2.75 mmol/g based on nitrogen analysis) in dimethylformamide (4 mL) was added 4-chloroaniline (32 mg, 0.25 mmol) and the mixture was stirred at room temperature overnight To the resulting suspension was added macroporous triethylammonium methylpolystyrene carbonate (250 mg, 2.8-3.5 mmol/g based on nitrogen elemental analysis) and Amberlyst 15 (650 mg) as scavengers and stirred for 2 hours (in case of acidic or basic final products the corresponding scavenger was not added). The resulting suspension was filtered and evaporated under reduced pressure. The residue was triturated with a mixture of MeOH:ethyl ether and the precipitate collected by filtration to yield the title compound as a white solid (80 mg, 78%).

ESI/MS m/e: 495 ([M+H]⁺, C₂₆H₂₇ClN₄O₄).

Retention Time (min.): 11.0.

### Example 6-53

The compounds of this invention were synthesized from the title compound of Preparation 2 following the procedure of example 5 and using the corresponding reactant respectively. The ESI/MS data, HPLC retention times and yields are summarised in the following table.

TABLE 15

| | | ESI/MS | | |
|---|---|---|---|---|
| | Molecular | m/e | Retention | |
| Example | Formula | [M + H]⁺ | Time (min.) | Yield % |
| 6 | C₂₇H₂₇F₃N₄O₅ | 545 | 11.1 | 35 |
| 7 | C₂₇H₂₇N₅O₄ | 486 | 10.2 | 60 |
| 8 | C₂₇H₂₉N₅O₅ | 504 | 9.1 | 48 |
| 9 | C₂₈H₃₃N₇O₄ | 532 | 9.5 | 57 |
| 10 | C₂₇H₃₀N₄O₅ | 491 | 10.3 | 76 |
| 11 | C₂₇H₃₀N₄O₄ | 475 | 10.7 | 38 |
| 12 | C₂₈H₃₀N₄O₅ | 503 | 10.1 | 64 |
| 13 | C₂₉H₃₂N₄O₆ | 533 | 10.8 | 52 |
| 14 | C₂₇H₂₇F₃N₄O₄ | 529 | 11.1 | 75 |
| 15 | C₃₀H₃₄ClN₅O₄ | 564 | 11.2 | 37 |
| 16 | C₃₀H₃₆N₄O₄ | 517 | 11.5 | 45 |
| 17 | C₃₂H₃₅N₅O₄ | 554 | 10.6 | 55 |
| 18 | C₃₇H₄₁N₅O₄ | 620 | 11.5 | 40 |
| 19 | C₂₈H₃₂N₄O₅ | 505 | 9.7 | 93 |
| 20 | C₂₈H₃₁ClN₄O₄ | 524 | 10.6 | 73 |
| 21 | C₃₃H₃₂N₄O₅ | 565 | 11.0 | 80 |
| 22 | C₂₈H₂₉N₅O₄ | 500 | 9.9 | 63 |
| 23 | C₂₆H₂₉N₅O₆S | 540 | 9.3 | 50 |
| 24 | C₂₆H₂₈N₄O₅ | 477 | 9.5 | 28 |
| 25 | C₃₂H₃₂N₄O₄ | 537 | 11.4 | 61 |
| 26 | C₃₃H₃₄N₄O₅ | 567 | 11.2 | 41 |
| 27 | C₃₂H₃₇N₅O₆ | 588 | 10.5 | 39 |
| 28 | C₃₆H₃₉N₅O₆ | 638 | 10.5 | 64 |
| 29 | C₃₁H₃₅N₅O₆ | 574 | 10.3 | 39 |
| 30 | C₃₁H₃₇N₅O₆S | 608 | 10.1 | 76 |
| 31 | C₃₇H₄₀N₄O₄ | 605 | 11.5 | 66 |
| 32 | C₃₂H₃₈N₄O₅ | 559 | 10.9 | 39 |
| 33 | C₃₀H₃₄N₄O₆ | 547 | 10.6 | 47 |
| 34 | C₃₃H₃₉N₇O₄ | 598 | 8.4 | 70 |
| 35 | C₃₆H₃₉N₅O₄ | 606 | 9.7 | 71 |
| 36 | C₃₁H₃₅N₅O₅ | 558 | 10.3 | 65 |
| 37 | C₂₉H₃₆N₅O₄ | 519 | 7.0 | 44 |
| 38 | C₂₉H₃₂Cl₂N₆O₄ | 599 | 10.7 | 35 |
| 39 | C₃₁H₃₃ClN₆O₄S | 621 | 11.3 | 56 |
| 40 | C₂₈H₃₁N₅O₅ | 518 | 9.4 | 60 |
| 41 | C₃₁H₃₃N₅O₄ | 540 | 10.7 | 52 |
| 42 | C₂₆H₂₇IN₄O₄ | 587 | 11.2 | 44 |
| 43 | C₂₈H₃₂N₄O₅ | 505 | 9.8 | 62 |
| 44 | C₂₉H₃₄N₄O₅ | 519 | 10.1 | 88 |
| 45 | C₃₂H₃₅N₅O₆ | 586 | 10.1 | 65 |
| 46 | C₃₃H₄₁N₅O₅ | 588 | 7.3 | 79 |
| 47 | C₂₈H₃₂N₄O₆ | 521 | 9.0 | 87 |
| 48 | C₂₉H₃₄N₄O₇ | 551 | 8.6 | 84 |
| 49 | C₂₉H₃₂N₄O₅ | 517 | 10.0 | 63 |
| 50 | C₃₂H₃₇N₅O₄ | 556 | 11.0 | 60 |
| 51 | C₂₆H₂₈N₄O₅ | 477 | 10.1 | 44 |
| 52 | C₂₉H₃₂N₄O₆ | 533 | 10.3 | 91 |
| 53 | C₂₈H₃₀N₄O₆ | 519 | 10.2 | 57 |
| 54 | C₂₈H₃₀N₄O₆ | 519 | 9.7 | 85 |

### Example 54

### (4-{2-[4-(2,4-Dioxo-1,3-dipropyl-2,3,4,5-tetrahydro-1H-pyrrolo[3,2-d]pyrimidin-6-yl)phenoxy]acetylamino}phenyl) acetic Acid

To a suspension of the title compound of Example 33 (33 mg, 0.06 mmol) in methanol (0.3 mL) was added NaOH 2N (0.3 mL) and the mixture was heated at 50° C. for 1 hour. The mixture was cooled to room temperature and acetic acid was added until acidic pH was observed. The resulting precipitate was collected by filtration and dried to yield the title compound (13 mg, 42%) as a white solid.

ESI/MS m/e: 519 ([M+H]⁺, C₂₈H₃₀N₄O₆).

Retention Time (min.): 9.7.

General Procedure for the Synthesis of Examples 55-76

The reaction took place in a sealed tube under argon atmosphere. Usually 50 mg of the title compound of Preparation 3 were used and 2 mL of those amines that are liquid and 160 equivalents of those amines that are solid. In all reactions a catalytic amount of sodium cyanide was added. In case of liquid amines the reaction mixture was heated at the boiling temperature of the amine and in the case of solid amines 2 mL of anhydrous dioxane were added and heated to the boiling point of dioxane. The reactions were followed by TLC and when no more starting material was left, the mixture was cooled to room temperature and usually the final product was isolated by filtration of the corresponding precipitate which was washed with ethyl ether. Occasionally the reaction mixture was concentrated under reduced pressure and the residue chromatographed on silica-gel (dichloromethane:methanol). The title compounds were crystallized in mixtures of MEOH:H₂O.

### Example 55

### N-(2-Aminoethyl)-2-[4-(1,3-dimethyl-2,4-dioxo-2,3,4,5 tetrahydro-1H-pyrrolo[3,2-d]pyrimidin-6-yl)phenoxy]acetamide

Obtained as a white solid (33%) from the title compound of Preparation 3 and ethylenediamine following the procedure described above.

δ ¹H NMR (DMSO): 7.85 (d, 2H), 7.01 (d, 2H), 6.63 (s, 1H), 4.52 (s, 2H), 3.41 (s, 3H), 3.25 (s, 3H), 3.12 (m, 2H), 2.50 (m, 2H).

### Example 56

### N-(4-Bromophenyl)-2-[4-(1,3-dimethyl-2,4-dioxo-2,3,4,5-tetrahydro-1H pyrrolo[3,2-d]pyrimidin-6-yl)phenoxy]acetamide

Obtained as a brown solid (15%) from the title compound of Preparation 3 and 4-bromoaniline following the procedure described above.

δ ¹H NMR (DMSO): 7.89 (d, 2H), 7.19 (d, 2H), 7.00 (d, 2H), 6.67 (s, 1H), 6.57 (m, 2H), 4.61 (s, 2H), 3.49 (s, 3H), 3.33 (s, 3H).

### Example 57

### 2-[4-(1,3-Dimethyl-2,4-dioxo-2,3,4,5-tetrahydro-1H-pyrrolo[3,2-d]pyrimidin-6-yl)phenoxy]-N-phenylacetamide

Obtained as a brown solid (74%) from the title compound of Preparation 3 and aniline following the procedure described above.

m.p.: >300° C.

δ ¹H NMR (DMSO): 12.30 (bs, 1H), 10.22 (bs, 1H), 7.88 (d, 2H), 7.66 (d, 2H), 7.34 (m, 2H), 7.09 (m, 3H), 6.62 (s, 1H), 4.78 (s, 2H), 3.42 (s, 3H), 3.27 (s, 3H).

ESI/MS (m/e,%): 405 [(M+1)⁺, 46].

### Example 58

### 2-[4-(1,3-Dimethyl-2,4-dioxo-2,3,4,5-tetrahydro-1H-pyrrolo[3,2-d]pyrimidin-6-yl)phenoxy]-N-4-fluorophenyl) Acetamide

Obtained as a white solid (10%) from the title compound of Preparation 3 and 4 fluoroaniline following the procedure described above.

m.p.: >300° C.

δ ¹H NMR (DMSO): 12.50 (bs, 1H), 10.36 (bs, 1H), 8.08 (d, 2H), 7.87 (m, 2H), 7.38 (m, 2H), 7.28 (d, 2$, 6.83 (s, 1H, 5.00 (s, 2H), 3.53 (s, 3H), 3.46 (s, 3H).

ESI/MS (m/e,%): 423 [(M+1)⁺, 100].

### Example 59

### 1,3-Dimethyl-6-{4-[2-(4-methylpiperazin-1-yl)-2-oxo-ethoxy]phenyl}-1,5-dihydropyrrolo[3,2-d]pyrimidine-2,4-dione

Obtained as a brown solid (72%) from the title compound of Preparation 3 and 1-methylpiperazine following the procedure described above.

m.p.: >275° C.

δ ¹H NMR (DMSO): 7.84 (d, 2H), 6.97 (d, 2H), 6.57 (s, 1H), 4.88 (s, 2H), 3.42 (s, 3H), 3.27 (s, 3H), 2.51 (m, 2H), 2.35 (m, 2H), 2.27 (m, 2H), 2.13 (m, 2H).

ESI/MS (m/e,%): 412 [(M+1)⁺, 100].

### Example 60

### 1,3-Dimethyl-6-[4-(2-morpholin-4-yl-2-oxoethoxy)phenyl]-1,5-dihydropyrrolo[3,2-d]pyrimidine-2,4-dione

Obtained as a brown solid (27%) from the title compound of Preparation 3 and morpholine following the procedure described above.

m.p.: >300° C.

δ ¹H NMR (DMSO): 12.42 (bs, 1H), 8.01 (d, 2H), 7.16 (d, 2H), 6.80 (s, 1$), 5.07 (s, 2H), 3.76 (m, 4H), 3.63 (m, 4H), 3.59 (s, 3H), 3.43 (s, 3$.

ESI/MS (m/e,%): 398 (M⁺, 42).

### Example 61

### 6-{4-[2-3,4-Dihydro-1H-isoquinolin-2-yl)-2-oxoethoxy]phenyl}-1,3-dimethyl-1,5-dihydropyrrolo[3,2-d]pyrimidine-2,4-dione

Obtained as a white solid (32%) from the title compound of Preparation 3 and 1,2,3,4-tetrahydro isoquinoline following the procedure described above.

m.p.: >280° C.

δ ¹H NMR (DMSO): 12.14 (bs, 1H), 7.71 (d, 2H), 7.07 (m, 4H), 6.89 (d, 2H), 6.50 (s, 1H), 4.86 (s, 2H), 3.56 (m, 2H), 3.35 (m, 2H), 3.29 (s, 3H), 3.13 (s, 3H), 2.72 (m, 1H), 2.39 (m, 1H).

ESI/MS (m/e,%): 444 (M⁺, 34).

### Example 62

### N-Cyclopentyl-2-[4-(1,3-dimethyl-2,4-dioxo-2,3,4,5-tetrahydro-1H-pyrrolo[3,2-d]pyrimidin-6-yl)phenoxy]acetamide

Obtained as a white solid (81%) from the title compound of Preparation 3 and cyclopentylamine following the procedure described above.

m.p.: >270° C.

δ ¹HNMR (DMSO): 8.02 (d, 2H), 7.18 (d, 2H), 6.69 (s, 1H), 4.68 (s, 2H), 4.27 (m, 1H), 3.60 (s, 3H), 3.45 (s, 3H), 2.03-1.60 (m, 8H).

ESI/MS (m/e,%): 396 (M⁺, 18).

### Example 63

### N-(4-Acetylphenyl)-2-[4-(1,3-dimethyl-2,4-dioxo-2,3,4,5-tetrahydro-1H-pyrrolo[3,2-d]pyrimidin-6-yl)phenoxy]acetamide

Obtained as a brown solid (24%) from the title compound of Preparation 3 and acetanilide following the procedure described above.

m.p.: >300° C.

δ ¹H NMR (DMSO): 8.03 (d, 2H), 7.93 (d, 2H), 7.85 (d, 2H), 7.15 (d, 2H), 6.68 (s, 1H), 4.89 (s, 2H), 3.49 (s, 3H), 3.33 (s, 3H), 2.60 (s, 3H).

ESI/MS (m/e,%): 446 (M⁺, 35).

### Example 64

### N-(1H-Benzoimidazol-2-yl)-2-[4-(1,3-dimethyl-2,4-dioxo-2,3,4,5-tetrahydro-1H-pyrrolo[3,2-d]pyrimidin-6-yl)phenoxy]acetamide

Obtained as a brown solid (84%) from the title compound of Preparation 3 and 2-aminobenzimidazole following the procedure described above.

m.p.: >287° C. (decomposition).

δ ¹H NMR (DMSO): 12.12 (bs, 1H), 7.83 (d, 2H), 7.40 (m, 2H), 7.04 (m, 4H), 6.80 (bs, 1H), 6.58 (s, 1H), 6.04 (bs, 1H), 4.88 (s, 2H), 3.38 (s, 3H), 3.22 (s, 3H).

### Example 65

### N-(4-Cyanophenyl)-2-[4-(1,3-dimethyl-2,4-dioxo-2,3,4,5-tetrahydro-1H-pyrrolo[3,2-d]pyrimidin-6-yl)phenoxy]acetamide

Obtained as a brown solid (13%) from the title compound of Preparation 3 and 4-aminobenzonitrile following the procedure described above.

m.p.: 263-265° C.

δ ¹H NMR (DMSO): 12.18 (bs, 1H), 10.50 (bs, 1H), 7.79 (m, 6H), 7.05 (m, 2H), 6.60 (s, 1H), 4.77 (s, 2H), 3.37 (s, 3H), 3.24 (s, 3H).

### Example 66

### 6-{4-[2-(3,4-Dihydro-2H-quinolin-1-yl)-2-oxoethoxy]phenyl}-1,3-dimethyl-1,5-dihydropyrrolo[3,2-d]pyrimidine-2,4-dione

Obtained as a yellow solid (47%) from the title compound of Preparation 3 and 1,2,3,4-tetrahydroquinoline following the procedure described above.

δ¹H NMR (CDCl₃): 11.60 (s, 1H), 7.62 (d, 2H), 7.10 (m, 4H), 6.78 (d, 2H), 5.20 (s, 1H), 4.79 (s, 2H), 3.76 (m, 2H), 3.73 (s, 3H), 3.23 (s, 3H), 2.60 (m, 2H 2H).

### Example 67

### 2-[4-(1,3-Dimethyl-2,4-dioxo-2,3,4,5-tetrahydro-1H-pyrrolo[3,2-d]pyrimidin-6-yl)phenoxy]-N-[1,3,4]thiadiazol-2-ylacetamide

Obtained as a brown solid (29%) from the title compound of Preparation 3 and 2-amino-1,3,4-thiadiazole following the procedure described above.

m.p.: >300° C. (decomposition).

δ ¹H NMR (DMSO): 9.23 (s, 1H), 8.64 (s, 1H), 7.93 (d, 2H), 7.26 (s, 1H), 7.12 (d, 2H), 6.70 (s, 1H), 5.03 (s, 2H), 3.49 (s, 3H), 3.33 (s, 3H).

### Example 68

### 1,3-Dimethyl-6-{4-[2-oxo-2-(4-phenylpiperazin-1-yl)ethoxy]phenyl}-1,5-dihydropyrrolo[3,2-d]pyrimidine-2,4-dione

Obtained as a while solid (20%) from the title compound of Preparation 3 and 1-phenylpiperazine following the procedure described above.

m.p.: >270° C. (decomposition).

δ ¹H NMR (DMSO): 11.25 (s, 1H), 7.76 (d, 2H), 7.28 (m, 3H), 7.02 (d, 2H), 6.91 (m, 2H), 6.18 (s, 1H), 4.80 (s, 2H), 3.78 (m, 4H), 3.53 (s, 3H), 3.49 (s, 3H), 3.47 (m, 4H).

### Example 69

### 2-[4-(1,3-Dimethyl-2,4-dioxo-2,3,4,5-tetrahydro-1H-pyrrolo[3,2-d]pyrimidin-6-yl)phenoxy]-N-(4-nitrophenyl)acetamide

Obtained as a yellow solid (15% overall) from the title compound of Preparation 1 and 4-nitroaniline following the procedure of example 1.

m.p.: 228-230° C.

δ¹H NMR (DMSO): 12.30 (bs, 1H), 10.80 (bs, 1H), 8.31 (d, 2H), 7.95 (m, 4H), 7.13 (d, 2H), 6.69 (s, 1H), 4.91 (s, 2H), 3.51 (s, 3H), 3.31 (s, 3H).

### Example 70

### 6-(4-{2-[4-(4-Fluorophenyl)piperazin-1-yl]-2-oxoethoxy}phenyl)-1,3-dimethyl-1,5-dihydropyrrolo[3,2-d]pyrimidine-2,4-dione

Obtained as a white solid (50%) from the title compound of Preparation 3 and 1-(4-fluorophenyl)piperazine following the general procedure described above.

m.p.: >265° C. (decomposition).

δ ¹H NMR (DMSO): 12.30 (bs, 1H), 7.84 (d, 2H), 7.04 (m, 6H), 6.63 (s, 1H), 4.95 (s, 2H), 3.62 (m, 4H), 3.42 (s, 3H), 3.26 (s, 3H), 3.14 (m, 2H), 3.07 (m, 2H).

ESI/MS (m/e,%): 491 (M⁺, 100).

### Example 71

### 6-{4-[2-(4-Benzylpiperazin-1-yl)-2-oxoethoxy]phenyl}-1,3-dimethyl-1,5 dihydropyrrolo[3,2-4-pyrimidine-2,4-dione

Obtained as an off-white solid (40%) from the title compound of Preparation 3 and 1-benzylpiperazine following the general procedure described above.

m.p.: 170-172° C.

δ ¹H NMR (DMSO): 12.05 (bs, 1H), 7.64 (d, 2H), 7.14 (s, 5H), 6.7.8 (d, 2H), 6.38 (s, 1H), 4.68 (s, 2H), 3.20 (m, 8H), 2.24 (m, 2H), 2.16 (m, 2H).

ESI/MS (m/e,%): 487 (M⁺, 100).

### Example 72

### 6-(4-{2-[4-(2-Methoxyphenyl)piperazin-1-yl]-2-oxoethoxy}phenyl)-1,3-dimethyl-1,5-dihydro-pyrrolo[3,2-d]pyrimidine-2,4-dione

Obtained as a brown solid (83%) from the title compound of Preparation 3 and 12-methoxyphenyl)piperazine following the general procedure described above.

m.p.: >295° C. (decomposition).

δ ¹H NMR (DMSO): 12.21 (bs, 1H), 7.76 (m, 2H), 6.86 (m, 6H), 6.52 (s, 1H), 4.88 (s, 2H), 3.76 (s, 3H), 3.58 (m, 4H), 3.38 (s, 3H), 3.22 (s, 3H), 2.49 (m, 4H).

ESI/MS (m/e,%): 503 (, 100).

### Example 73

### 6-(4-{2-[4-(4-Methoxyphenyl)piperazin-1-yl]-2-oxoethoxy}phenyl)-1,3-dimethyl-1,5-dihydropyrrolo[3,2-d]pyrimidine-2,4-dione

Obtained as a white solid (23%) from the title compound of Preparation 3 and 1-(4-methoxyphenyl)piperazine following the general procedure described above.

m.p.: 269-271° C.

δ ¹H NMR (DMSO): 12.38 (bs, 1H), 7.96 (d, 2H), 7.09 (d, 2H), 7.05 (d, 2H), 6.96 (d, 2H), 6.74 (s, 1H), 5.06 (s, 2H), 3.81 (s, 3H), 3.73 (m, 4H), 3.54 (s, 3H), 3.38 (s, 3H), 3.19 (m, 2H), 3.11 (m, 4H).

ESI/MS (m/e,%): 503 (M⁺, 100).

### Example 74

### 1,3-Dimethyl-6-(4-{2-oxo-2-[4-(3-trifluoromethylphenyl)piperazin-1-yl]ethoxy}phenyl)-1,5-dihydropyrrolo[3,2-d]pyrimidine-2,4-dione

Obtained as a white solid (50%) from the title compound of Preparation 3 and 1-(3-trifluoromethylphenyl)piperazine following the general procedure described above.

m.p.: >275° C. (decomposition) δ ¹H NMR (DMSO): 7.77 (m, 2H), 7.40 (m, 1H), 7.28 (m, 2H), 7.15 (d, 1H), 6.94 (m, 3H), 4.88 (s, 2H), 4.65 (s, 1H), 3.68 (s, 3H), 3.30 (m, 1H).

ESI/MS (m/e,%): 541 (M⁺, 100).

### Example 75

### 1 Dimethyl-6-{4-[2-oxo-2-(4-pyridin-2-yl-piperazin-1-yl)ethoxy]phenyl}-1,5 dihydropyrrolo[3,2-d]pyrimidine-2,4-dione

Obtained as a white solid (42%) from the title compound of Preparation 3 and 1-pyridin-2-ylpiperazine following the general procedure described above.

m.p.: >260° C. (decomposition).

δ ¹H NMR (DMSO): 8.14 (d, 1H), 7.84 (d, 2H), 7.57 (m, 1H), 7.01 (d, 2H), 6.88 (m, 1H), 6.68 (m, 1H), 6.60 (s, 1H), 4.91 (s, 2H), 3.60-3.26 (m, 14H).

ESI/MS (m/e,%): 474 (M⁺, 100).

### Example 76

### 1,3-Dimethyl-6-{4-[2-oxo-2-(4-pyrimidin-2-ylpiperazin-1-yl)ethoxy]phenyl}-1,5 dihydropyrrolo[3,2-d]pyrimidine-2,4-dione

Obtained as a white solid (60%) from the title compound of Preparation 3 and 2-piperazin-1-ylpyrimidine following the general procedure described above.

m.p.: >275° C. (decomposition).

δ ¹H NMR (DMSO): 12.27 (bs, 1H), 8.41 (d, 2H), 7.85 (d, 2H), 7.03 (d, 1H), 6.69 (t, 1H), 6.63 (s, 1H), 4.96 (s, 2H), 3.83 (m, 2H), 3.76 (m, 2H), 3.57 (m, 4H), 3.43 (s, 3H), 3.27 (s, 3H).

### Examples 77-86

The compounds of this invention were synthesized from the title compound of Preparation 4 following the procedure of example 5 and using the corresponding reactant respectively. The ESI/MS data, HPLC retention times and yields are summarised in the following table.

TABLE 16

| | | ESI/MS | | |
|---|---|---|---|---|
| | Molecular | m/e | Retention | |
| Example | Formula | [M + H]⁺ | Time (min.) | Yield % |
| 77 | C₂₄H₂₄N₄O₄ | 433 | 15.3 | 33 |
| 78 | C₂₅H₂₆N₄O₄ | 447 | 16.2 | 36 |
| 79 | C₂₅H₂₄N₄O₄ | 445 | 16.0 | 37 |
| 80 | C₂₃H₂₁FN₄O₄ | 437 | 15.0 | 30 |
| 81 | C₂₁H₂₀N₄O₅ | 409 | 13.6 | 50 |
| 82 | C₂₃H₂₁ClN₄O₄ | 452 | 16.0 | 55 |
| 83 | C₂₄H₂₄N₄O₄ | 433 | 15.5 | 60 |
| 84 | C₂₄H₂₄N₄O₅ | 449 | 14.8 | 40 |
| 85 | C₂₃H₂₂N₄O₄ | 419 | 14.7 | 43 |
| 86 | C₂₈H₂₉N₅O₄ | 500 | 9.5 | 20 |

### Examples 87-89

The compounds of this invention were synthesized from the title compound of Preparation 5 following the procedure of example 5 and using the corresponding reactant respectively. The ESI/MS data, HPLC retention times and yields are summarised in the following table.

TABLE 17

| | | ESI/MS | | |
|---|---|---|---|---|
| | Molecular | m/e | Retention | |
| Example | Formula | [M + H]⁺ | Time (min.) | Yield % |
| 87 | C₂₄H₂₄N₄O₄ | 433 | 10.6 | 10 |
| 88 | C₂₈H₃₁N₅O₄ | 502 | 10.9 | 24 |
| 89 | C₂₅H₂₃N₅O₄ | 458 | 9.4 | 35 |

### Examples 90-107

The compounds of this invention were synthesized from the title compound of Preparation 6 following the procedure of example 5 and using the corresponding reactant respectively. The ESI/MS data, HPLC retention times and yields are summarised in the following table.

TABLE 18

| | | ESI/MS | | |
|---|---|---|---|---|
| | Molecular | m/e | Retention | |
| Example | Formula | [M + H]⁺ | Time (min.) | Yield % |
| 90 | C₂₄H₂₄N₄O₄ | 433 | 9.6 | 51 |
| 91 | C₂₄H₂₃FN₄O₄ | 450 | 9.7 | 48 |
| 92 | C₂₅H₂₅ClN₄O₄ | 480 | 10.0 | 60 |
| 93 | C₂₇H₂₈N₄O₄ | 473 | 9.8 | 45 |
| 94 | C₂₈H₃₁N₅O₄ | 502 | 9.9 | 40 |
| 95 | C₂₈H₃₀FN₅O₄ | 520 | 10.0 | 62 |
| 96 | C₂₇H₂₈N₄O₆ | 505 | 10.2 | 39 |
| 97 | C₂₉H₃₂N₄O₅ | 517 | 9.3 | 47 |
| 98 | C₃₀H₃₁N₅O₄ | 526 | 9.9 | 50 |
| 99 | C₃₀H₂₈N₄O₄ | 509 | 10.9 | 86 |
| 100 | C₃₅H₃₆N₄O₄ | 577 | 10.9 | 88 |
| 101 | C₃₀H₃₄N₄O₅ | 531 | 10.3 | 66 |
| 102 | C₂₈H₃₀N₄O₆ | 519 | 9.9 | 49 |
| 103 | C₃₄H₃₅N₅O₄ | 578 | 8.6 | 65 |
| 104 | C₂₉H₂₉ClN₆O₄S | 593 | 10.8 | 44 |
| 105 | C₂₉H₂₉N₅O₄ | 512 | 10.1 | 58 |
| 106 | C₂₄H₂₃IN₄O₄ | 528 | 10.2 | 31 |
| 107 | C₃₀H₃₃N₅O₄ | 620 | 11.5 | 44 |

### Examples 108-116

The compounds of this invention were synthesized from the title compound of Preparation 7 following the procedure of example 5 and using the corresponding reactant respectively. The ESI/MS data, HPLC retention times and yields are summarised in the following table.

TABLE 19

| | | ESI/MS | | |
|---|---|---|---|---|
| | Molecular | m/e | Retention | |
| Example | Formula | [M + H]⁺ | Time (min.) | Yield % |
| 108 | C₂₄H₂₃FN₄O₄ | 451 | 10.8 | 78 |
| 109 | C₂₄H₂₄N₄O₄ | 433 | 10.7 | 65 |
| 110 | C₂₄H₂₃BrN₄O₄ | 512 | 11.6 | 72 |
| 111 | C₂₇H₂₈N₄O₄ | 473 | 11.0 | 99 |
| 112 | C₂₅H₂₆N₄O₄ | 447 | 10.5 | 47 |
| 113 | C₂₆H₂₈N₄O₄ | 461 | 10.8 | 88 |
| 114 | C₂₈H₃₁N₅O₄ | 502 | 11.1 | 73 |
| 115 | C₂₉H₃₃N₅O₄ | 516 | 7.8 | 69 |
| 116 | C₃₀H₃₃N₅O₄ | 528 | 10.3 | 23 |

### Example 117

### N-Cyclopentyl-2-{4-[1-(3-methoxyropyl)-3-methyl-2,4-dioxo-2,3,4,5-tetrahydro-1H-pyrrolo[3,2-d]pyrimidin-yl]phenoxy}acetamide

The compound of this invention was synthesized from the title compound of Preparation 8 and cyclopentylamine following the general procedure described for examples 55-76.

m.p.(MeOH/H₂O): 234-236° C.

δ (DMSO): 7.83 (d, 2H), 6.99 (d, 2H), 6.56 (s, 1H), 4.48 (s, 2H), 4.05 (m, 1H), 3.93 (t, 2H), 3.39 (s, 3H), 3.37 (s, 3M, 1.92-1.07 (m, 10H).

### Example 118

### 2-{4-[1-(3-Methoxypropyl)-3-methyl-2,4-dioxo-2,3,4,5-tetrahydro-1H-pyrrolo[3,2-d]pyrimidin-6-yl]phenoxy}-N-phenylacetamide

The compound of this invention was synthesized from the title compound of Preparation 8 and aniline following the general procedure described for examples 55-76.

m.p.(MeOH/H₂O): >251° C. (dec.)

δ ¹H NMR (CDCl₃): 7.71 (d, 2H), 7.60 (d, 2H), 7.38 (m, 3H), 7.15 (m, 2H), 6.34 (s, 1H), 4.70 (s, 2H), 4.10 (in, 2H), 3.48 (m, 2H) 3.37 (s, 3H), 2.05 (m, 2H).

ESI/MS (m/e,%): 462 (M⁺, 100).

### Examples 119-120

The compounds of this invention were synthesized from the title compound of Preparation 9 following the procedure of example 5 and using the corresponding reactant respectively. The ESI/MS data, HPLC retention times and yields are summarised in the following table.

TABLE 20

| | | ESI/MS | | |
|---|---|---|---|---|
| | Molecular | m/e | Retention | |
| Example | Formula | [M + H]⁺ | Time (min.) | Yield % |
| 119 | C₂₅H₂₆N₄O₄ | 447 | 9.9 | 51 |
| 120 | C₂₉H₃₃N₅O₄ | 516 | 10.2 | 64 |

### Examples 121-123

The compounds of this invention were synthesized from the title compound of Preparation 10 following the procedure of example 5 and using the corresponding reactant respectively. The ESI/MS data, HPLC retention times and yields are summarised in the following table.

TABLE 21

| | | ESI/MS | | |
|---|---|---|---|---|
| | Molecular | m/e | Retention | |
| Example | Formula | [M + H]⁺ | Time (min.) | Yield % |
| 121 | C₂₆H₂₆N₄O₆ | 491 | 9.7 | 57 |
| 122 | C₂₉H₃₂N₄O₅ | 517 | 9.8 | 40 |
| 123 | C₂₈H₃₁N₅O₅ | 518 | 9.1 | 48 |

### Example 124

### N-(4-Bromophenyl)-2-[4-(2,4-dioxo-1-propyl-2,3,4,5-tetrahydro-1H-pyrrolo[3,2-d]pyrimidin-6-yl)phenoxy]acetamide

Obtained as a white solid (11%) from the title compound of Preparation 10 and 4-bromoaniline following the procedure of Example 4.

m.p.: 276-278 (dec.)

δ ¹H NMR (DMSO): 12.00 (bs, 1H), 10.60 (bs, 1H), 10.22 (bs, 1H), 7.86 (d, 2H), 7.60 (d, 2H), 7.40 (d, 2H), 7.09 (d, 2H), 6.60 (s, 1H), 4.78 (s, 2H), 3.80 (t, 2H), 1.64 (m, 2H), 0.90 (t, 3H).

### Example 125

### 2-[4-(2,4-Dioxo-1-propyl-2,3,4,5-tetrahydro-1H-pyrrolo[3,2-d]pyrimidin-6-yl)phenoxy]-N-4-fluorophenyl)acetamide

Obtained as a white solid (56%) from the title compound of Preparation 10 and 4-fluoroaniline following the procedure of Example 4.

m.p.: 306-308° C. (dec.)

δ ¹H NMR (DMSO): 12.20 (bs, 1H), 10.78 (bs, 1H), 10.15 (bs, 1H), 7.85 (d, 2H), 7.65 (dd, 2H), 7.16 (t, 2H), 7.05 (d, 2H), 6.61 (s, 1H), 4.73 (s, 2H), 1.64 (m, 2H), 0.90 (t, 3H).

### Example 126-130

The compounds of this invention were synthesized from the title compound of Preparation 11 following the procedure of example 5 and using the corresponding reactant respectively. The ESI/MS data, HPLC retention times and yields are summarised in the following table.

TABLE 22

| | | ESI/MS | | |
|---|---|---|---|---|
| | Molecular | m/e | Retention | |
| Example | Formula | [M + H]⁺ | Time (min.) | Yield % |
| 126 | C₂₆H₂₈N₄O₆ | 493 | 9 | 90 |
| 127 | C₂₆H₂₇FN₄O₆ | 511 | 9.1 | 85 |
| 128 | C₂₆H₂₇BrN₄O₆ | 573 | 9.9 | 84 |
| 129 | C₃₀H₃₅N₅O₆ | 562 | 9.4 | 82 |
| 130 | C₂₉H₃₂N₄O₆ | 533 | 9.3 | 94 |

### Examples 131-135

The compounds of this invention were synthesized from the title compound of Preparation 12 following the procedure of example 5 and using the corresponding reactant respectively. The ESI/MS data, HPLC retention times and yields are summarised in the following table.

TABLE 23

| | | ESI/MS | | |
|---|---|---|---|---|
| | Molecular | m/e | Retention | |
| Example | Formula | [M + H]⁺ | Time (min.) | Yield % |
| 131 | C₂₈H₂₈N₄O₄ | 485 | 10.6 | 88 |
| 132 | C₂₈H₂₇FN₄O₄ | 503 | 10.6 | 79 |
| 133 | C₂₈H₂₇BrN₄O₄ | 564 | 11.2 | 68 |
| 134 | C₃₂H₃₅N₅O₄ | 554 | 10.8 | 92 |
| 135 | C₃₁H₃₂N₄O₄ | 525 | 10.8 | 95 |

### Example 136

### 2-[4-(7-Chloro-1,3-dimethyl-2,4-dioxo-2,3,4,5-tetrahydro-1H-pyrrolo[3,2-d]pyrimidin-6-yl)phenoxy]-N-(4-cyanophenyl)acetamide

Obtained as a white solid (42%) from the title compound of Preparation 13 and 4-aminobenzonitrile following the procedure of example 5.

ESI/MS m/e: 463 (M+H]⁺, C₂₃H₁₈ClN₅O₄).

Retention Time (min.): 16.7.

### Examples 137-138

The compounds of this invention were synthesized from the title compound of Preparation 14 following the procedure of example 5 and using the corresponding reactant respectively. The ESI/MS data, HPLC retention times and yields are summarised in the following table.

TABLE 24

| | | ESI/MS | | |
|---|---|---|---|---|
| | Molecular | m/e | Retention | |
| Example | Formula | [M + H]⁺ | Time (min.) | Yield % |
| 137 | C₂₆H₂₇BrN₄O₄ | 540 | 20.1 | 55 |
| 138 | C₂₆H₂₆BrFN₄O₄ | 558 | 20.1 | 62 |

### Examples 139-149

The compounds of this invention were synthesized from the title compound of Preparation 15 following the procedure of example 5 and using the corresponding reactant respectively. The ESI/MS data, HPLC retention times and yields are summarised in the following table.

TABLE 25

| | | ESI/MS | | |
|---|---|---|---|---|
| | Molecular | m/e | Retention | |
| Example | Formula | [M + H]⁺ | Time (min.) | Yield % |
| 139 | C₂₆H₂₆ClFN₄O₄ | 512 | 20.0 | 65 |
| 140 | C₂₆H₂₇ClN₄O₄ | 494 | 19.9 | 72 |
| 141 | C₂₆H₂₆BrClN₄O₄ | 573 | 21.0 | 35 |
| 142 | C₂₆H₂₆Cl₂N₄O₄ | 529 | 21.3 | 74 |
| 143 | C₂₆H₂₆Cl₂N₄O₄ | 529 | 20.0 | 82 |
| 144 | C₂₆H₂₆ClFN₄O₄ | 512 | 20.2 | 78 |
| 145 | C₂₇H₂₈ClFN₄O₄ | 526 | 19.7 | 80 |
| 146 | C₂₇H₂₉ClN₄O₅ | 525 | 19.7 | 48 |
| 147 | C₂₇H₂₉ClN₄O₄ | 509 | 19.7 | 70 |
| 148 | C₂₇H₂₉ClN₄O₄ | 509 | 20.5 | 60 |
| 149 | C₂₆H₂₆ClFN₄O₄ | 512 | 20.2 | 58 |

### Examples 150-158

The compounds of this invention were synthesized from the title compound of Preparation 16 following the procedure of example 5 and using the corresponding reactant respectively. The ESI/MS data, HPLC retention times and yields are summarised in the following table.

TABLE 26

| | | ESI/MS | | |
|---|---|---|---|---|
| | Molecular | m/e | Retention | |
| Example | Formula | [M + H]⁺ | Time (min.) | Yield % |
| 150 | C₂₃H₂₂N₄O₅ | 435 | 10.1 | 58 |
| 151 | C₂₃H₂₁FN₄O₅ | 453 | 10.2 | 47 |
| 152 | C₂₄H₂₃ClN₄O₅ | 359 | 8.4 | 61 |
| 153 | C₂₆H₂₆N₄O₅ | 475 | 10.4 | 43 |
| 154 | C₂₇H₂₉N₅O₅ | 504 | 10.5 | 60 |
| 155 | C₂₄H₂₁N₅O₅ | 460 | 10.0 | 72 |
| 156 | C₂₃H₂₁BrN₄O₅ | 513 | 11.0 | 70 |
| 157 | C₂₉H₃₂N₄O₆ | 533 | 9.8 | 51 |
| 158 | C₂₈H₃₁N₅O₆ | 534 | 9.1 | 46 |

### Examples 159-168

The compounds of this invention were synthesized from the title compound of Preparation 17 following the procedure of example 5 and using the corresponding reactant respectively. The ESI/MS data HPLC retention times and yields are summarised in the following table.

TABLE 27

| | | ESI/MS | | |
|---|---|---|---|---|
| | Molecular | m/e | Retention | |
| Example | Formula | [M + H]⁺ | Time (min.) | Yield % |
| 159 | C₂₃H₂₂N₄O₅ | 435 | 10.0 | 49 |
| 160 | C₂₃H₂₁FN₄O₅ | 452 | 10.1 | 65 |
| 161 | C₂₄H₂₃ClN₄O₅ | 482 | 10.3 | 58 |
| 162 | C₂₆H₂₆N₄O₅ | 475 | 10.1 | 41 |
| 163 | C₂₇H₂₉N₅O₅ | 504 | 10.1 | 45 |
| 164 | C₂₄H₂₁N₅O₅ | 460 | 9.8 | 59 |
| 165 | C₂₃H₂₁BrN₄O₅ | 514 | 10.9 | 70 |
| 166 | C₂₆H₂₆N₄O₇ | 507 | 9.6 | 44 |
| 167 | C₂₉H₃₂N₄O₆ | 533 | 9.5 | 50 |
| 168 | C₂₈H₃₁N₅O₆ | 534 | 8.8 | 39 |

### Examples 169-174

The compounds of this invention were synthesized from the title compound of Preparation 18 following the procedure of example 5 and using the corresponding reactant respectively. The ESI/MS data, HPLC retention times and yields are summarised in the following table.

TABLE 28

| | | ESI/MS | | |
|---|---|---|---|---|
| | Molecular | m/e | Retention | |
| Example | Formula | [M + H]⁺ | Time (min.) | Yield % |
| 169 | C₂₇H₃₀N₄O₄ | 475 | 11.6 | 59 |
| 170 | C₃₀H₃₄N₄O₄ | 515 | 11.9 | 34 |
| 171 | C₃₁H₃₇N₅O₄ | 544 | 11.9 | 39 |
| 172 | C₂₈H₃₁ClN₄O₄ | 523 | 11.9 | 47 |
| 173 | C₂₇H₂₉FN₄O₄ | 493 | 11.7 | 59 |
| 174 | C₂₈H₃₂N₄O₅ | 505 | 11.5 | 52 |

### Examples 175-179

The compounds of this invention were synthesized from the title compound of Preparation 19 following the procedure of example 5 and using the corresponding reactant respectively. The ESI/MS data, HPLC retention times and yields are summarised in the following table.

TABLE 29

| | | ESI/MS | | |
|---|---|---|---|---|
| | Molecular | m/e | Retention | |
| Example | Formula | [M + H]⁺ | Time (min.) | Yield % |
| 175 | C₂₃H₂₂N₄O₄ | 419 | 8.8 | 85 |
| 176 | C₂₃H₂₁FN₄O₄ | 437 | 9 | 90 |
| 177 | C₂₃H₂₁BrN₄O₄ | 498 | 9.8 | 55 |
| 178 | C₂₇H₂₉N₅O₄ | 488 | 9.3 | 59 |
| 179 | C₂₆H₂₆N₄O₄ | 459 | 9.2 | 75 |

### Examples 180-184

The compounds of this invention were synthesized from the title compound of Preparation 20 following the procedure of example 5 and using the corresponding reactant respectively. The ESI/MS data, HPLC retention times and yields are summarised in the following table.

TABLE 30

| | | ESI/MS | | |
|---|---|---|---|---|
| | Molecular | m/e | Retention | |
| Example | Formula | [M + H]⁺ | Time (min.) | Yield % |
| 180 | C₂₈H₃₂N₄O₄ | 489 | 10.8 | 78 |
| 181 | C₂₈H₃₁FN₄O₄ | 507 | 10.9 | 77 |
| 182 | C₂₈H₃₁BrN₄O₄ | 569 | 11.4 | 65 |
| 183 | C₃₂H₃₉N₅O₄ | 558 | 11.1 | 85 |
| 184 | C₃₁H₃₆N₄O₄ | 529 | 11.1 | 82 |

### Examples 185-189

The compounds of this invention were synthesized from the title compound of Preparation 21 following the procedure of example 5 and using the corresponding reactant respectively. The ESI/MS data, HPLC retention times and yields are summarised in the following table.

TABLE 31

| | | ESI/MS | | |
|---|---|---|---|---|
| | Molecular | m/e | Retention | |
| Example | Formula | [M + H]⁺ | Time (min.) | Yield % |
| 185 | C₂₈H₃₂N₄O₄ | 489 | 10.9 | 47 |
| 186 | C₂₈H₃₁FN₄O₄ | 506 | 10.9 | 50 |
| 187 | C₂₈H₃₁BrN₄O₄ | 568 | 11.5 | 48 |
| 188 | C₃₂H₃₉N₅O₄ | 558 | 11.4 | 37 |
| 189 | C₃₁H₃₆N₄O₄ | 529 | 11.5 | 30 |

### Examples 190-192

The compounds of this invention were synthesized from the title compound of Preparation 22 following the procedure of example 5 and using the corresponding reactant respectively. The ESI/MS data, HPLC retention times and yields are summarised in the following table.

TABLE 32

| | | ESI/MS | | |
|---|---|---|---|---|
| | Molecular | m/e | Retention | |
| Example | Formula | [M + H]⁺ | Time (min.) | Yield % |
| 190 | C₃₂H₃₂N₄O₄ | 537 | 11.0 | 93 |
| 191 | C₃₂H₃₁FN₄O₄ | 555 | 11.0 | 20 |
| 192 | C₃₆H₃₉N₅O₄ | 606 | 11.4 | 80 |

### Examples 193-196

The compounds of this invention were synthesized from the title compound of Preparation 23 following the procedure of example 5 and using the corresponding reactant respectively. The ESI/MS data, HPLC retention times and yields are following table:

TABLE 33

| | | ESI/MS | | |
|---|---|---|---|---|
| | Molecular | m/e | Retention | |
| Example | Formula | [M + H]⁺ | Time (min.) | Yield % |
| 193 | C₂₇H₃₀N₄O₃ | 459 | 10.3 | 59 |
| 194 | C₂₇H₂₉FN₄O₃ | 477 | 10.4 | 52 |
| 195 | C₃₁H₃₇N₅O₃ | 528 | 10.7 | 35 |
| 196 | C₃₀H₃₄N₄O₃ | 499 | 10.9 | 21 |

### Examples 197-199

The compounds of this invention were synthesized from the title compound of Preparation 24 following the procedure of example 5 and using the corresponding reactant respectively. The ESI/MS data, HPLC retention times and yields are summarised in the following table.

TABLE 34

| | | ESI/MS | | |
|---|---|---|---|---|
| | Molecular | m/e | Retention | |
| Example | Formula | [M + H]⁺ | Time (min.) | Yield % |
| 197 | C₂₇H₂₈N₄O₃ | 457 | 11.9 | 66 |
| 198 | C₃₁H₃₅N₅O₃ | 526 | 12.2 | 52 |
| 199 | C₃₀H₃₂N₄O₃ | 497 | 12.3 | 60 |

### Examples 200-204

The compounds of this invention were synthesized from the title compound of Preparation 25 following the procedure of example 5 and using the corresponding reactant respectively. The ESI/MS data, HPLC retention times and yields are summarised in the following table.

TABLE 35

| | | ESI/MS | | |
|---|---|---|---|---|
| | Molecular | m/e | Retention | |
| Example | Formula | [M + H]⁺ | Time (min.) | Yield % |
| 200 | C₂₈H₃₂N₄O₄ | 489 | 10.6 | 62 |
| 201 | C₂₈H₃₁FN₄O₄ | 507 | 10.6 | 72 |
| 202 | C₃₂H₃₉N₅O₄ | 558 | 11.0 | 48 |
| 203 | C₃₁H₃₆N₄O₄ | 529 | 11.1 | 69 |
| 204 | C₃₄H₄₁N₅O₄ | 584 | 11.3 | 62 |

### Examples 205-209

The compounds of this invention were synthesized from the title compound of Preparation 26 following the procedure of example 5 and using the corresponding reactant respectively. The ESI/MS data, HPLC retention times and yields are summarised in the following table.

TABLE 36

| | | ESI/MS | | |
|---|---|---|---|---|
| | Molecular | m/e | Retention | |
| Example | Formula | [M + H]⁺ | Time (min.) | Yield % |
| 205 | C₂₅H₂₆N₄O₃ | 431 | 10.3 | 61 |
| 206 | C₂₅H₂₅FN₄O₃ | 449 | 10.4 | 53 |
| 207 | C₂₅H₂₅BrN₄O₃ | 510 | 11.1 | 55 |
| 208 | C₂₉H₃₃N₅O₃ | 500 | 10.7 | 54 |
| 209 | C₂₈H₃₀N₄O₃ | 471 | 10.7 | 48 |

### Example 210

### 6-[4-(3-Phenyl[1,2,4]oxadiazol-5-ylmethoxy)phenyl]-1,3-dipropyl-1,5-dihydropyrrolo[3,2-d]pyrimidine-2,4-dione

a) To a mixture of the title compound of Preparation 2 (400 mg, 1.03 mmol), N-(3-dimethylaminopropyl)-N′-ethyl-carbodiimide hydrochloride (237 mg, 1.24 mmol) and 1-hydroxybenzotriazole (167 mg, 1.24 mmol) in dimethylformamide (15 mL) was added triethylamine (288 μL, 2.06 mmol) and N-hydroxybenzamidine (168 mg, 1.24 mmol). The mixture was stirred at room temperature overnight.

The solvent was evaporated under reduced pressure and the residue was partitioned between dichloromethane and a 1 M aqueous solution of citric acid. The organic phase was separated, washed with a saturated aqueous solution of sodium bicarbonate, dried (Na₂SO₄) and evaporated under reduced pressure. The residue was triturated with ethyl ether and the precipitate was collected by filtration to yield the title compound as a yellow solid (144 mg, 28%).

b) A stirred solution of the above compound (140 mg, 0.277 mmol) in toluene (50 mL) was refluxed using a Dean-Stark apparatus for 20 hours. The solvent was evaporated under reduced pressure, the residue was triturated with ethyl ether and the precipitate was collected by filtration to yield the title compound as a yellow solid (90 mg, 67%).

δ ¹H NMR (CDCl₃): 10.3 (bs, 1H), 8.1 (m, 2H), 7.7 (d, 2H), 7.5 (d, 2H), 7.2 (d, 1H), 7.1 (d, 1H), 6.2 (s, 1H), 5.4 (s, 2H), 4.0 (m, 4H), 1.7 (m, 4H), 0.9 (dt, 6H).

ESI/MS (m/e,%): 486 (M⁺, 100).

Retention Time (min.): 11.4.

### Example 211

### 6-{4-[2-oxo-2-{[amino(4-fluorophenyl)methylenediamino]-oxy}ethoxy]phenyl}-1,3-dipropyl-1,5-dihydropyrrolo[3,2-d]pyrimidine-2,4-dione

Obtained as a white solid (88%) from the title compound of Preparation 2 and 4-fluoro-N-hydroxybenzamidine following the procedure a) of Example 210.

δ¹H NMR (DMSO): 12.2 (s, 1H), 7.8 (dd, 4H), 7.3 (m, 2H), 7.0 (d, 4H), 6.6 (s, 1H), 5.0 (s, 2H), 3.8 (m, 4H), 1.6 (m, 4H), 0.9 (m, 6H).

ESI/MS (m/e,%): 522 (M⁺, 100).

Retention Time (min.): 10.1.

### Example 212

### 6-{4-[3-(4-Fluorophenyl) [1,2,4]oxadiazol-5-ylmethoxy]phenyl}-1,3-dipropyl-1,5 dihydropyrrolo[3,2-d]pyrimidine-2,4-dione

Obtained as a white solid (83%) from the title compound of Example 211 following the procedure b) of Example 210.

δ ¹H NMR (DMSO): 12.2 (s, 1H), 8.1 (dd, 2H), 7.9 (d, 2H), 7.4 (t, 2H), 7.1 (d, 2H), 6.7 (s, 1H), 5.6 (s, 2H), 3.8 (m, 4H), 1.6 (m, 4H), 0.9 (dt, 6H).

ESI/MS (m/e,%): 504 (M⁺, 100).

Retention Time (min.): 11.4.

### Example 213

### 1,3-Dipropyl-6-[4-(3-pyridin-4-yl[1,2,4]oxadiazol-5-ylmethoxy)phenyl]-1,5-dihydropyrrolo[3,2-d]pyrimidine-2,4-dione

Obtained as a white solid (87%) from the title compound of Preparation 2 and N-hydroxyisonicotinamidine following the same procedure of Example 210.

δ ¹H NMR (DMSO): 12.2 (bs, 1H), 8.8 (d, 1H), 8.7 (d, 1H), 7.9 (m, 3H), 7.7 (d, 1H), 7.2 (d, 1H), 7.1 (d, 1H), 6.7 (s, 1H), 5.7 (s, 2H), 3.9 (m, 4H), 1.6 (m, 4H), 0.9 (dt, 6H).

ESI/MS (m/e,%): 487 (M⁺, 100).

Retention Time (min.): 10.4.

### Example 214

### 6-4-(Benzooxazol-2-ylmethoxy)phenyl]-1,3-dipropyl-1,5-dihydropyrrolo[3,2-d]pyrimidine-2,4-dione

A stirred solution of the title compound of Example 51 (134 mg, 0.28=mol) and p-toluensulfonic acid (48 mg, 0.28 mmol) in toluene (10 mL) was refluxed using a Dean-Stark apparatus for 5 hours. The solvent was evaporated under reduced pressure, the residue was partitioned between dichlorometane and a saturated aqueous solution of sodium bicarbonate. The organic phase was separated, washed with brine, dried (MgSO₄) and evaporated under reduced pressure. The residue was triturated with ethyl ether and the precipitate was collected by filtration to yield the title compound as a white solid (82 mg, 64%).

δ ¹H NMR (CDCl₃): 10.9 (s, 1H), 7.7 (m, 3H), 7.5 (m, 1H), 7.3 (dd, 2H), 7.1 (d, 2H), 6.1 (s, 1H), 5.3 (s, 2H), 3.9 (m, 4H), 1.7 (dq, 4H), 0.9 (dt, 6H).

ESI/MS (m/e,%): 459 (M⁺, 100).

Retention Time (min.): 10.9.

### Example 215

### 6-[4-(5-Phenyl-4,5-dihydrooxazol-2-ylmethoxy)phenyl]-1,3-dipropyl-1,5-dihydropyrrolo[3,2-d]pyrimidine-2,4-dione

A solution of the title compound of Example 43 (60 mg, 0.119 mmol) in thionyl chloride (173 μL) was stirred at room temperature for 1 hour. The resulting solution was poured into water and a yellow solid precipitated. A suspension of the above solid in water was treated with a 2 N aqueous solution of sodium hydroxide until alkaline pH. The solid was collected by filtration and dried to yield the title compound as a yellow solid (35 mg, 60%).

ESI/MS (m/e,%): 487 (M⁺, 100).

Retention Time (min.): 10.7.

### Example 216

### 6-{4-(4-Methyl-5-phenyl-4,5-dihydrooxazol-2-ylmethoxy)phenyl]-1,3-dipropyl-1,5 dihydropyrrolo[3,2-d]pyrimidine-2,4-dione

Obtained as a yellow solid (45%) from the title compound of Example 44 following procedure of Example 215.

ESI/MS (m/e,%): 501 (M⁺, 100).

Retention Time (min.): 11.0.

### Example 217

### 6-[4-(7-Benzyl-1-oxa-3,7-diazaspiro[4.5]dec-2-en-2-ylmethoxy)phenyl]-1,3-dipropyl-1,5-dihydropyrrolo[3,2-d]pyrimidine-2,4-dione

Obtained as a white solid (33%) from the title compound of Example 46 following the procedure described in Example 215:

ESI/MS (m/e,%): 570 (M⁺, 100).

Retention Time (min.): 7.3.

### Example 218

### 1,3-Dipropyl-[4-(quinoln-2-ylmethoxy)phenyl]-1,5-dihydropyrrolo[3,2-d]pyrimidine-2,4-dione

a) A mixture of p-hydroxybenzaldehyde (17.02 g, 0.139 mmol), 2-chloromethylquinoline (24.76 g, 0.139 mmol), potassium carbonate (57.64 g, 0.417 mmol) and potassium iodide (2.17 g, 0.013 mmol) in methyl isobutyl ketone (515 mL) was refluxed for 20 h After cooling to room temperature, the inorganic salts were filtered and the solvent was evaporated under reduced pressure. The residue was partitioned between dichlorometane and water, the aqueous phase extracted with dichloromethane and the organic phase washed with water and brine, dried (MgSO₄) and evaporated under reduced pressure. The residue was triturated with ethyl ether and the precipitate was collected by filtration to yield the 4-(quinolin-2-ylmethoxy)benzaldehyde as a yellow solid (25.62 g, 70%).

m.p.: 72.0° C.

b) The title compound was obtained as a yellow solid (560 mg, 61%) from 6-methyl-5-nitro-1,3-dipropyl-1H pyrimidine-2,4-dione (1.0 g, 3.92 mmol) and 4 (quinolin-2-ylmethoxy)benzaldehyde (1.13 g, 4.31 mmol) following the same procedure described in Preparation 2.

δ ¹H NMR (CDCl₃): 10.5 (s, 1H), 8.3 (d, 2H), 7.7 (m, 6H), 7.1 (d, 2H), 6.2 (s, 1H), 5.5 (s, 2H), 3.9 (m, 4H), 1.7 (m, 4H), 0.9 (dt, 6H).

ESI/MS (m/e,%): 469 (M⁺, 100).

Retention Time (min.): 11.3.

### Examples 219-226

The Compounds of this invention were synthesized from the title compound of Preparation 2 following the procedure of example 5 and using the corresponding reactant respectively. The ESI/MS data, HPLC retention times and yields are summarised in the following table.

TABLE 37

| | | ESI/MS | | |
|---|---|---|---|---|
| | Molecular | m/e | Retention | |
| Example | Formula | [M + H]⁺ | Time (min.) | Yield % |
| 219 | C₂₅H₂₇N₅O₄ | 462 | 10.1 | 54 |
| 220 | C₂₅H₂₇N₅O₅ | 478 | 9.5 | 36 |
| 221 | C₂₆H₂₉N₅O₄ | 476 | 10.5 | 26 |
| 222 | C₂₅H₂₇N₅O₄ | 462 | 93 | 70 |
| 223 | C₂₆H₂₉N₅O₅ | 492 | 10.0 | 36 |
| 224 | C₂₆H₂₉N₅O₄ | 476 | 7.6 | 40 |
| 225 | C₃₁H₃₄F₃N₅ O₄ | 598 | 11.0 | 42 |
| 226 | C₃₀H₃₄Cl N₅O₄ | 566 | 11.0 | 60 |

### Examples 227-229

The compounds of this invention were synthesized from the title compound of Preparation 2 following the procedure of example-5a and using the corresponding reactant respectively. The ESI/MS data and yields are summarised in the following table.

TABLE 38

| | | ESI/MS | |
|---|---|---|---|
| | Molecular | m/e | |
| Example | Formula | [M + H]⁺ | Yield % |
| 227 | C₂₄H₂₆N₆O₄ | 463 | 54 |
| 228 | C₂₆H₃₀N₆O₆ | 523 | 25 |
| 229 | C₂₃H₂₆N₆O₄ | 450 | 37 |

(Example 227) δ ¹H NMR (DMSO): 12.33 (bs, 1H), 11.05 (bs, 1H), 9.41 (s, 1H), 8.52 (m, 2H), 7.97 (d, 2H), 6.76 (s, 1H), 5.01 (s, 2H), 3.95 (m, 4H), 1.70 (m, 4H), 1.00 (m, 6H).

(Example 228) δ ¹H NMR (DMSO): 12.43 (bs, 1H), 11.04 (bs, 1H), 8.06 (d, 2H), 7.20 (m, 3H), 6.58 (bs, 1H), 5.08 (s, 2H), 4.08 (m, 10H), 1.80 (m, 4H), 1.07 (m, 6H).

(Example 229) δ ¹H NMR (DMSO): 12.27 (bs, 1H), 8.97 (d, 1H), 8.11 (d, 2H), 7.03 (d, 2H), 6.67 (s, 1H), 6.03 (d, 1H), 5.84 (s, 2H), 5.44 (s, 2H), 3.90 (m, 4H), 1.60 (m, 4H), 0.90 (m, 6H).

### Examples 230-239

The compounds of this invention were synthesized: from the title compound of Preparation 4 following the procedure of example 5 and using the corresponding reactant respectively. The ESI/MS data, HPLC retention times and yields are summarised in the following table.

TABLE 39

| | | ESI/MS | | |
|---|---|---|---|---|
| | Molecular | m/e | Retention | |
| Example | Formula | [M + H]⁺ | Time (min.) | Yield % |
| 230 | C₂₆H₂₆Cl N₅O₄ | 509 | 9.8 | 18 |
| 231 | C₂₇H₂₆F₃N₅O₄ | 542 | 10.0 | 58 |
| 232 | C₂₆H₂₆Br N₅O₄ | 552 | 9.9 | 33 |
| 233 | C₂₇H₂₈N₄O₅ | 489 | 8.4 | 40 |
| 234 | C₂₈H₂₇N₅O₄ | 498 | 9.1 | 17 |
| 235 | C₃₃H₃₂N₄O₄ | 549 | 10.4 | 27 |
| 236 | C₂₇H₂₇Cl N₄O₅ | 524 | 9.1 | 45 |
| 237 | C₂₅H₂₄Cl₂N₆O₄ | 543 | 9.2 | 68 |
| 238 | C₂₇H₂₅Cl N₆O₄S | 566 | 10.1 | 52 |
| 239 | C₂₇H₂₅N₅O₄ | 484 | 9.4 | 63 |

### Examples 240-242

The compounds of this invention were synthesized from the title compound of Preparation 3 following the procedure of example 55 and using the corresponding reactant respectively. The ESI/MS data, HPLC retention times and yields are summarised in the following table.

TABLE 40

| | | ESI/MS | |
|---|---|---|---|
| | Molecular | m/e | |
| Example | Formula | [M + H]⁺ | Yield % |
| 240 | C₂₈H₂₇FN₄O₅ | 519 | 40 |
| 241 | C₂₂H₂₁N₅O₄ | 419 | 71 |
| 242 | C₂₃H₂₇N₅O₆ | 469 | 42 |

(Example 240) δ ¹H NMR (DMSO): 12.29 (bs, 1H), 8.13 (dd, 2H), 7.85 (d, 2H), 7.40 (m, 2H), 7.00 (d, 2H), 6.65 (s, 1H), 4.92 (d, 2H), 4.37 (d, 1H), 3.92 (d, 1H 1H), 3.47 (m, 1H), 3.43 (s, 3H), 3.27 (s, 3H), 2.82 (m, 1H), 1.84 (m, 2H), 1.61 (m, 1H), 1.41 (m, 1H).

(Example 241) δ ¹H NMR (DMSO): 12.30 (bs, 1H), 8.79 (m, 1H), 8.50 (m, 1H), 7.89 (d, 2H), 725 (d, 2H), 7.01 (d, 2H), 6.66 (d, 1H), 4.68 (s, 2H), 4.38 (d, 2H), 3.43 (s, 3H), 327 (s, 3H).

(Example 242) δ ¹H NMR (DMSO): 12.32 (bs, 1H), 7.89 (d, 2H), 7.05 (d, 2H), 6.66 (s, 1H), 4.96 (s, 2H), 4.10 (m, 2H), 3.40 (m, 14H), 1.25 (s, 3H).

### Examples 243-246

The compounds of this invention were synthesized from the title compound of Preparation 6 following the procedure of example 5 and using the corresponding reactant respectively. The ESI/MS data, HPLC retention times and yields are summarised in the following table.

TABLE 41

| | | ESI/MS | | |
|---|---|---|---|---|
| | Molecular | m/e | Retention | |
| Example | Formula | [M + H]⁺ | Time (min.) | Yield % |
| 243 | C₃₀H₃₄N₄O₅ | 531 | 10.3 | 70 |
| 244 | C₂₇H₂₈Cl₂N₆O₄ | 571 | 9.8 | 40 |
| 245 | C₂₄H₂₅N₅O₅ | 463 | 8.9 | 65 |
| 246 | C₂₉H₃₀F₃N₅O₄ | 570 | 10.4 | 26 |

### Example 247-253

The compounds of this invention were synthesized from the title compound of Preparation X following the procedure of example 5 and using the corresponding reactant respectively. The ESI/MS data, HPLC retention times and yields are summarised in the following table.

TABLE 42

| | | ESI/MS | | |
|---|---|---|---|---|
| | Molecular | m/e | Retention | |
| Example | Formula | [M + H]⁺ | Time (min.) | Yield % |
| 247 | C₂₁H₂₄N₄O₅ | 412 | 6.6 | 50 |
| 248 | C₂₂H₂₇N₅O₄ | 425 | 4.7 | 41 |
| 249 | C₁₉H₂₂N₄O₅ | 386 | 6.1 | 36 |
| 250 | C₂₈H₃₁N₅O₅ | 518 | 8.4 | 29 |
| 251 | C₂₈H₃₁N₅O₄ | 502 | 5.7 | 44 |

(Example 252) δ ¹H NMR (DMSO): 12.15 (bs, 1H), 11.18 (bs, 1H), 10.27 (bs, 1H), 7.91 (d, 2H), 7.76 (m, 2H), 7.54 (m, 4H), 6.29 (s, 1H), 4.83 (s, 2H), 3.91 (m, 2H), 1.66 (m, 2H), 0.95 (t, 3H).

(Example 253)δ ¹H NMR (DMSO): 12.03 (bs, 1H), 11.20 (bs, 1H), 10.25 (bs, 1H), 7.81 (d, 2H), 7.61 (d, 2H), 7.50 (d 2H), 7.02 (d, 2H), 6.19 (s, 1H), 4.74 (s, 2H), 3.80 (m, 2H), 1.55 (m, 2H), 0.86 (t, 3H).

### Example 254

### 6-{4-2-Oxo-(4-phenylpiperazin-1-yl ethoxy]phenyl}-1-propyl-1,5 dihydropyrrolo p3,2-pyrimidine-2,4-dione

Obtained as a white solid (2%) from the title compound of Preparation 10 and 1-phenyl piperazine following the procedure of example 5.

m.p.(MeOH/H₂O): 280-282° C.

δ ¹H NM (DMSO): 12.19 (s, 1H), 10.78 (s, 1H), 7.81 (d, 2H), 7.22 (m, 2H), 6.96 (m, 4H), 6.80 (t, 1H), 6.60 (s, 1H), 4.92 (s, 2H), 3.78 (m, 2H), 3.60 (m, 4H), 3.15 (m, 4H), 1.66 (m, 2H), 0.90 (t, 3H).

ESI/MS (m/e,%): 487 (M⁺, 33).

### Examples 255-257

The compounds of this invention were synthesized from the title compound of Preparation 28 following the procedure of example 5 and using the corresponding reactant respectively. The ESI/MS data, HPLC retention times and yields are summarised in the following table.

TABLE 43

| | | ESI/MS | | |
|---|---|---|---|---|
| | Molecular | m/e | Retention | |
| Example | Formula | [M + H]⁺ | Time (min.) | Yield % |
| 255 | C₃₂H₃₇FN₆O₅ | 605 | 9.8 | 80 |
| 256 | C₃₂H₃₈N₆O₅ | 587 | 6.5 | 61 |
| 257 | C₃₃H₃₇F₃N₆O₅ | 655 | 7.5 | 39 |

### Example 258

### Pyrazin-2-yl-carbamic Acid 4-(2,4-dioxo-1,3-dipropyl-2,3,4,5 tetrahydro-1H-pyrrolo[3,2-d]pyrimid in-6-yl)benzyl Ester

a) To a solution of triphosgene (87 mg, 0.29 mmol) in anhydrous dioxane (5 mL) under argon atmosphere was slowly added at room temperature a solution of 2-aminopyrazine (84 mg, 0.89 mmol) and triethylamine (0.24 mL, 1.76 mmol) in dioxane (5 mL). The mixture was sired at room temperature for 1 hour.

b) Then the title compound of Preparation 30 was added to the above reaction mixture (100 mg, 0.29 mmol) and the solution was stirred 48 hours at room temperature. The mixture was evaporated under reduced pressure and the residue was partitioned between dichloromethane and a saturated aqueous solution of sodium bicarbonate. The organic phase was separated, washed with water and brine, dried (Na₂SO₄) and evaporated under reduced pressure. The resulting crude was purified by flash column chromatography on silica-gel (dichloromethane/MeOH 95:5) to yield the title compound as a white solid (25 mg, 19%).

m.p.(MeOH): 267-270° C.

δ ¹H NMR (DMSO): 12.56 (s, 1H), 10.83 (s, 1H), 9.27 (s, 1H), 8.50 (m, 2H), 8.11 (d, 2H), 7.66 (d, 2H), 6.95 (s, 1H), 5.40 (s, 2H), 4.02 (m, 4H), 1.75 (m, 6H).

### Example 259

### (2,6-Dimethoxy-pyrimidin-4-yl)-carbamic Acid 4-(2,4-dioxo-1,3-dipropyl-2,3,4,5-tetrahydro-1H-pyrrolo[3,2-d]pyrimidin-6-yl)benzyl Ester

Obtained as a white solid (20%) from the title compound of Preparation 30 and 4-amino-2,6-dimethoxypyrimidine following the procedure of example 258.

m.p.(MeOH): 182-185° C. δ¹H NMR (DMSO): 12.5 (bs, 1H), 10.73 (s, 1H), 8.04 (d, 2H), 7.57 (d, 2H), 6.94 (s, 1H), 6.89 (s, 1H), 5.30 (s, 2H), 3.96 (m, 4H), 1.73 (m, 4H), 0.98 (m, 6H).

ESI/MS (m/e,%): 523; 342 (100).

### Example 260

### Pyridin-4-ylmethyl Carbamic Acid 4-(2,4-dioxo-1,3-dipropyl-2,3,4,5-tetrahydro-1H-pyrrolo[3,2-d]pyrimidin-6-yl)benzyl Ester

To a solution of 1,1′-carbonyldiimidazole (48 mg, 0.29 mmol) in pyridine (0.5 mL) under argon atmosphere was slowly added at 0° C. a solution of the title compound of Preparation 30 (100 mg, 0.29 mmol) in pyrimidine (1 mL). The mixture was stirred at room temperature for for 1 hour. Then the title compound of Preparation 30 was added (100 mg, 0.29 mmol) and the mixture was stirred 2 hours at 0° C. and 2 hours at room temperature. To the reaction mixture was slowly added 1-phenylpyperacine (162 mg, 0.29 mmol) and the mixture was stirred at room temperature overnight. The resulting solution was cooled to 4° C. and the precipitate was collected by filtration to yield the title compound as a white solid (51 mg, 33%).

m.p.(MeOH): 240-2420° C.

δ ¹H NMR (DMSO): 12.36 (bs, 1H), 7.90 (d, 2H), 7.42 (d, 2H), 7.03 (m, 2H), 6.95 (m, 2H), 6.73 (s, 1H), 5.11 (s, 2H), 3.85 (m, 4H), 3.53 (m, 4H), 3.04 (m, 4H), 1.67 (m, 2H), 1.56 (m, 2H), 0.88 (m, 6H).

### Example 261

### 4-(3-Chlorophenyl)piperazine-1-carboxylic Acid 4-(2,4-dioxo-1,3-dipropyl-2,3,4,5-tetrahydro-1H-pyrrolo[3,2-d]pyrimidin-6-yl)benzyl Ester

Obtained as a white solid (15%) from the title compound of Preparation 30 and 1-(3-Chloro phenyl)piperazine following the procedure of example 260.

m.p.(MeOH): 188-190° C.

δ ¹H NMR (DMSO): 12.36 (s, 1H), 7.91 (d, 2H), 7.42 (d, 2H), 7.21 (m, 1H), 6.88 (m, 2H), 6.74 (m, 2H), 5.11 (s, 2H), 3.86 (m, 4H), 3.18 (m, 4H), 1.40 (m, 6H).

ESI/MS (m/e,%): 476; 324 (100).

### Example 262

### (1H-Pyrazol-3-yl)carbamic Acid 4-(2,4-dioxo-1,3-dipropyl-2,3,4,5-tetrahydro-1H-pyrrolo[3,2-d]pyrimidin-6-yl)benzyl Ester

Obtained as a white solid (60%) from the title compound of Preparation 30 and 1H-pyrazol-3-ylamine following the procedure of example 260.

m.p.(MeOH): 210-213° C.

δ ¹H NMR (DMSO): 12.39 (bs, 1H), 7.93 (m, 4H), 7.49 (d, 2H), 6.76 (s, 1H), 5.85 (s, 1H), 5.51 (s, 1H), 5.33 (s, 2H), 3.86 (m, 4H), 1.75 (m, 4H), 0.88 (m, 6H).

### Example 263

### 4-(3-Trifluoromethylphenyl)piperazine-1-carboxylic Acid 4-(2,4-dioxo-1,3-dipropyl-2,3,4,5-tetrahydro-1H-pyrrolo[3,2-d]pyrimidin-6-yl)benzyl Ester

Obtained as a white solid (42%) from the title compound of Preparation 30 and 1-(3-trifluoro methylphenyl)piperazine following the procedure of example 260.

m.p.(MeOH): 232-233° C.

δ ¹H NMR (DMSO): 12.38 (s, 1H), 7.91 (m, 2H), 7.43 (m, 3H), 7.20 (m, 2H), 7.08 (m, 1H), 6.75 (s, 1H), 5.11 (s, 2H), 3.86 (m, 4H), 3.55 (m, 4H), 3.23 (m, 4H), 1.60 (m, 4H), 0.88 (m, 6H).

### Example 264

### Isoxazol-3-yl-carbamic Acid 4-(2,4-dioxo-1,3-dipropyl-2,3,4,5-tetrahydro-1H-pyrrolo[3,2-d]pyrimidin-6-yl)benzyl Ester

Obtained as a white solid (41%) from the title compound of Preparation 30 and isoxazol-3-ylamine following the procedure of example 260.

m.p.(MeOH): 168-171° C.

δ¹H NMR (DMSO): 12.40 (bs, 1H), 8.30 (m, 1H), 7.95 (d, 2N), 7.62 (s, 1H), 7.55 (d, 2H), 7.07 (s, 1H), 6.76 (s, 1H), 5.45 (s, 2H), 3.86 (m, 4H), 1.60 (m, 4H), 0.88 (m, 6H).

### Example 265-272

The compounds of this invention were synthesized from the title compound of Preparation 29 following the procedure of example 258 and using the corresponding reactant respectively. The ESI/MS data, melting points and yields are summarised in the following table.

TABLE 44

| | | ESI/MS | | |
|---|---|---|---|---|
| | Molecular | m/e | m.p. (° C.) | |
| Example | Formula | [M + H]⁺ | (MeOH) | Yield % |
| 265 | C₂₂H₁₉FN₄O₄ | 423 | — | 30 |
| 266 | C₂₃H₂₂N₄O₄ | 419 | — | 30 |
| 267 | C₂₂H₂₀N₄O₄ | 405 | — | 40 |
| 268 | C₂₁H₁₉N₅O₄ | 406 | 301 | 60 |
| 269 | C₂₂H₂₁N₅O₄ | 420 | 293 | 51 |
| 270 | C₂₀H₁₈N₄O₄S | 411 | 287 | 31 |
| 271 | C₂₀H₁₈N₄O₄S | 411 | 280 | 20 |
| 272 | C₂₀H₁₈N₄O₅ | 395 | 278 | 23 |

(Example 265) δ ¹H NMR (DMSO): 12.52 (bs, 1H), 9.91 (s, 1H), 8.00 (d, 2H), 7.55 (m, 2H), 7.20 (m, 2H), 6.83 (s, 1H), 5.24 (s, 2H), 3.50 (s, 3H), 3.33 (s, 3H).

(Example 266) δ ¹H NMR (DMSO): 12.53 (bs, 1H), 7.91 (d, 2H), 7.41 (d, 2H), 7.30 (m, 5H), 6.75 (s, 1H), 5.07 (s, 2H), 4.21 (d, 2H), 3.42 (s, 3H), 3.26 (s, 3H).

(Example 267) δ ¹H NMR (DMSO): 12.50 (bs, 1H), 9.85 (s, 1H), 8.00 (d, 2H), 7.53 (m, 4H), 7.33+(m, 2H), 7.05 (m, 1H), 6.81 (s, 1H), 5.23 (s, 2H), 3.49 (s, 3H).

### Example 273-274

The compounds of this invention were synthesized from the title compound of Preparation 30 following the procedure of example 260 and using the corresponding reactant respectively. The ESI/MS data and yields are summarised in the following table.

TABLE 45

| | | ESI/MS | |
|---|---|---|---|
| | Molecular | m/e | |
| Example | Formula | [M + H]⁺ | Yield % |
| 273 | C₂₆H₂₇N₅O₄ | 474 | 60 |
| 274 | C₂₅H₂₄N₄O₄ | 445 | 45 |

### Example 275

### Thiophen-2-yl-carbamic Acid 2-[4-(2,4-dioxo-1,3-dipropyl-2,3,4,5-tetrahydro-1H-pyrrolo[3,2-d]pyrimidin-6-yl)phenoxy]ethyl Ester

a) From the title compound of Preparation 2 following the procedure of Preparation 29c, 6-[4-2-hydroxyethoxy)phenyl]-1,341-propyl-1,5-dihydropyrrolo[3,2-d]pyrimidine-2,4-dione was obtained (70%) as a white solid.

δ ¹H NMR (DMSO): 12.04 (bs, 1H), 7.68 (d, 2H), 6.82 (d, 2H), 6.47 (d, 1H), 4.71 (t, 1H), 3.86 (m, 2H), 3.68 (m, 4H), 3.54 (m, 2H), 1.45 (m, 4H), 0.72 (m, 6H).

b) The title compound was obtained as a white solid (60%) from the above compound and 2-isocyanatothiophene following the procedure of example 258.

m.p.(MeOH/Et₂O): 223-225° C.

δ ¹H NMR (DMSO): 12.29 (bs, 1H), 10.86 (bs, 1H), 7.94 (d, 2H), 7.10 (d, 2H), 6.99 (dd, 1H), 6.87 (dd, 1H), 6.73 (s, 1H), 6.63 (dd, 1H), 4.52 (m, 2H), 4.35 (m, 2H), 3.93 (m, 4H), 1.69 (m, 4H), 0.95 (m, 6H).

### Example 276

### (4-Bromophenyl)carbamic Acid 2-[4-(2,4-dioxo-1,3-dipropyl-2,3,4,5-tetrahydro-1H-pyrrolo[3,2-d]pyrimidin-6-yl)phenoxy]ethyl ester

Obtained as a brownish solid (23%) from the title compound of Preparation 2 and 4-bromo-phenylisocyanate following the procedure of example 275.

m.p.(MeOH): 281° C. (dec.)

δ ¹H NMR (DMSO): 12.23 (bs, 1H), 9.99 (s, 1H), 7.88 (d, 2H), 7.46 (m, 4H), 7.04 (d, 2H), 6.66 (s, 1H), 4.44 (m, 2H), 4.30 (m, 2H), 3.86 (m, 4H), 1.62 (m, 4H), 0.90 (m, 6H).

### Example 277

### 1-[1-(2,6-Difluorophenyl)methanoyl]-3-[4-(2,4-dioxol dipropyl-2,3,4,5-tetrahydro-1H-pyrrolo[3,2-d]pyrimidin-yl)benzyl]urea

a) To a suspension of the title compound of Preparation 30 (0.48 g, 1.40 mmol) in dichloromethane (45 mL) was added methanesulfonyl chloride (545 μL, 7.04 mmol) and triethyl anine (981 μL, 7.04 mmol) and the mixture was stirred at room temperature for 5 hours. The solvent was evaporated under reduced pressure, the residue was triturated with dichloromethane and the resulting solid was filtered, washed with dichloromethane and dried to yield methanesulfonic acid 4-(2,4-dioxo-1,3-dipropyl-2,3,4,5-tetrahydro-1H-pyrrolo[3,2-d]pyrimidin-6-yl)benzyl ester (0.22 g, 37%) as a yellow solid.

b) To a suspension of the above compound (0.22 g, 0.52 mmol) in dimethylformamide (5.5 mL) under argon atmosphere, was added sodium azide (68 mg, 1.05 mmol) and the mixture was heated at 40° C. for 4 hours. The solvent was evaporated under reduced pressure, the residue was triturated with water and the resulting solid was filtrated, washed with water and diethyl ether and dried to yield 6-4-azidomethylphenyl)-1,3-dipropyl-1,5-dihydropyrrolo[3,2-d]pyrimidine-2,4-dione (0.15 g, 79%) as a yellow solid.

c) To a suspension of the above compound (0.15 g, 0.41 mmol) in tetrahydrofuran (2 mL) at 0° C., was added a solution of 1 M trimethyl phosphine in toluene (656 μL, 0.65 mmol) and the resulting solution was stirred at room temperature for 5 hours. Water (22 μL, 1.23 mmol) was added and the solution was stirred at room temperature for 18 hours. The solvent was evaporated under reduced pressure, the residue was triturated with dichloromethane and the resulting solid was filtrated, washed with dichloromethane and dried to yield 6-(4-aminomethylphenyl)-1,3-dipropyl-1,5-dihydropyrrolo[3,2-d]pyrimidine-2,4-dione (96 mg, 69%) as a yellow solid.

d) To a solution of the above compound (25 mg, 0.07 mmol) in dimethylformamide (1 mL) was added 2,6-difluorobenzoyl isocyanate (20 mg, 0.088 mmol) and the mixture was stirred at room temperature for 4 hours. Tris-2-aminoethyl)amine polystyrene (0.12 g, 0.44 mmol) was added and the mixture was stirred for 1 hour. After filtration, the solvent was evaporated under reduced pressure, the residue was triturated with a mixture of diethyl ether and dichloromethane and the resulting solid was filtrated, washed with diethyl ether and dried to yield the title compound (53%) as a yellow solid.

ESI/MS m/e: 524 ([M+H]⁺, C H₇F₂N₅O₄).

Retention Time (min.): 10.1.

### Example 278-279

The compounds of this invention were synthesized from the title compound of Preparation 2 following the procedure of example 214 and using the corresponding reactant respectively. The ESI/MS data, HPLC retention times and yields are summarised in the following table.

TABLE 46

| | | ESI/MS | | |
|---|---|---|---|---|
| | Molecular | m/e | Retention | |
| Example | Formula | [M + H]⁺ | Time (min.) | Yield % |
| 278 | C₂₆H₂₅FN₄O₄ | 477 | 11.0 | 63 |
| 279 | C₂₆H₂₇N₅O₃ | 458 | 9.6 | 67 |

### Example 280

### 1,3-Dimethyl-6-[4-(quinolin-2-ylmethoxy)phenyl]-1,5-dihydropyrrolo[3,2-d]pyrimidine-2,4-dione

Obtained as a white solid (50%) from 6-methyl-5-nitro-1,3 dimethyl-1H-pyrimidine-2,4-dione and 4-(quinolin-2-ylmethoxy)benzaldehyde following the procedure of example 218.

ESI/MS m/e: 413 ([M+H]⁺, C₂₄H₂₀N₄O₃).

Retention Time (min.): 9.7.

### Example 281

### 1,3-Dimethyl-6-4-(3-phenyl[1,2,4]oxadiazol-5-ylmethoxy)phenyl]-1,5 dihydropyrrolo[3,2-d]pyrimidine-2,4-dione

Obtained as a white solid-(60%) from the title compound of Preparation 4 and N-hydroxybenzamidine following the procedure of example 210.

ESI/MS m/e: 430 ([M+H]⁺, C₂₃H₁₉N₅O₄).

Retention Time (min.): 10.0.

### Example 282-284

The compounds of this invention were synthesized from the title compound of Preparation 6 following the procedure of example 210 and using the corresponding reactant respectively. The ESI/MS data, HPLC retention times and yields are summarised in the following table.

TABLE 47

| | | ESI/MS | | |
|---|---|---|---|---|
| | Molecular | m/e | Retention | |
| Example | Formula | [M + H]⁺ | Time (min.) | Yield % |
| 282 | C₂₅H₂₃N₅O₄ | 458 | 10.7 | 54 |
| 283 | C₂₅H₂₂FN₅O₄ | 476 | 10.9 | 43 |
| 284 | C₂₄H₂₁Cl N₄O₄ | 465 | 10.8 | 60 |

### Example 285

### 6-{4-[3-(4-Bromophenyl)[1,2,4]oxadiazol-5-ylmethoxy]-phenyl}-3-propyl-1,5-dihydropyrrolo[3,2-d]pyrimidine-2,4-dione

Obtained as a white solid (30%) from the title compound of Preparation 27 and 4-bromo-N-hydroxybenzamidine following the procedure of example 210.

δ ¹H NMR (DMSO): 12.13 (bs, 1H), 11.16 (bs, 1H), 7.96 (d, 2H), 7.85 (d, 4H), 7.15 (d, 2H), 6.24 (s, 1H), 5.67 (s, 2H), 3.83 (m, 2H), 1.58 (m, 2H), 0.88 (m, 3H).

### Example 286-289

The compounds of this invention were synthesized from the title compound of Preparation 19 following the procedure of example 210 and using the corresponding reactant respectively. The ESI/MS data, HPLC retention times and yields are summarised in the following table.

TABLE 48

| | | ESI/MS | | |
|---|---|---|---|---|
| | Molecular | m/e | Retention | |
| Example | Formula | [M + H]⁺ | Time (min.) | Yield % |
| 286 | C₂₄H₂₁N₅O₄ | 444 | 10.4 | 44 |
| 287 | C₂₄H₂₀FN₅O₄ | 462 | 10.5 | 26 |
| 288 | C₂₂H₁₉N₅O₄S | 450 | 10.0 | 55 |
| 289 | C₂₅H₂₃N₅O₄S | 490 | 10.9 | 58 |

### Example 290

### 6-{4-[(4-Bromophenylamino)methyl]phenyl}-1,3-dipropyl-1,5-dihydropyrrolo[3,2-d]pyrimidine-2,4-dione

a) To a solution of the title compound of Preparation 30 (200 mg, 0.59 mmol) in DMF (5 mL) was added CBr₄ (480 mg, 1.02 mmol) and the mixture was cooled to 0° C. Then a solution of triphenyl phosphine (270 mg, 1.02 mmol) in DME (2 mL) was added and the mixture was stirred at room temperature for 14 hours. The precipitate was collected by filtration and used in the next step without further purification.

b) To a solution of 4-bromoaniline (43 mg, 0.25 mmol) in ethanol (2 mL) was added K₂CO₃ (34 mg, 0.025 mmol) and the above bromide (20 mg, 0.05 mmol). The mixture was refluxed for 1 hour. The solvent was evaporated under reduced pressure, the residue was suspended in chloroform, the organic phase was washed with water, dried (Na₂SO₄) and evaporated. Flash column chromatography (chloroform:petroleum ether 9:1) provided the title compound as a brown solid (11 mg, 44%).

m.p.(MeOH): >250° C.

δ ¹H NMR (DMSO): 10.7 (bs, 1H), 7.72 (d, 2H), 7.42 (d, 2H), 7.24 (d, 2H), 6.50 (d, 2H), 6.24 (s, 1H), 4.36 (s, 2H), 3.95 (m, 4H), 1.75 (m, 4H), 0.95 (m, 6H).

### Example 291

### 6-(4-Phenylaminomethylphenyl)-1,3-dipropyl-1,5-dihydropyrrolo[3,2-d]pyrimidine-2,4-dione

Obtained as a brownish solid (64%) from the title compound of Preparation 30 and aniline following the procedure of example 290.

m.p.(MeOH): 201° C.

δ ¹H NMR (DMSO): 10.51 (bs, 1H), 7.70 (m, 1H), 7.46 (m, 1H), 7.24 (m, 4H), 6.75 (m, 3H), 6.24 (s, 1H), 4.39 (s, 2H), 3.96 (m, 4H), 1.70 (m, 4H), 1.00 (m, 6H).

The following examples illustrate pharmaceutical compositions according to the present invention and procedures for their preparation.

### Composition Example 1

50,000 capsules each containing 100 mg of active ingredient were prepared according to the following formulation:

| | | |
|---|---|---|
| Active ingredient | 5 | Kg |
| Lactose monohydrate | 10 | Kg |
| Colloidal silicone dioxide | 0.1 | Kg |
| Corn starch | 1 | Kg |
| Magnesium stearate | 0.2 | Kg |

Procedure

The above ingredients were sieved through a 60 mesh sieve, and were loaded into a suitable mixer and filled into 50,000 gelatine capsules.

### Composition Example 2

50,000 Tablets each containing 50 mg of active ingredient were prepared from the following formulation:

| | | |
|---|---|---|
| Active ingredient | 2.5 | Kg |
| Microcrystalline cellulose | 1.95 | Kg |
| Spray dried lactose | 9.95 | Kg |
| Carboxymethyl starch | 0.4 | Kg |
| Sodium stearyl fumarate | 0.1 | Kg |
| Colloidal silicon dioxide | 0.1 | Kg |

Procedure

AU the powders were passed through a screen with an aperture of 0.6 mm, then mixed in a suitable mixer for 20 minutes and compressed into 300 mg tablets using 9 mm disc and flat bevelled punches. The disintegration time of the tablets was about 3 minutes.

1. A 6-phenylpyrrolopyrimidinedione derivative of the formula (I), or a pharmaceutically acceptable salt thereof, 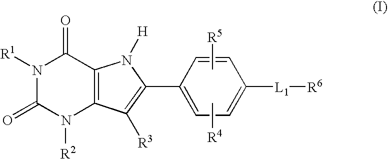
wherein:
R¹ and R² are the same or different and each represents hydrogen, a group of formula —(CH₂)ₙ—R⁷, or an alkyl group which is unsubstituted or substituted by one or more substituents selected from hydroxy, alkoxy, alkylthio, amino, mono- or di-alkylamino, hydroxycarbonyl, alkoxycarbonyl, acylamino, carbamoyl, alkylcarbamoyl, dihydroxyphosphoryloxy and dialkoxyphosphoryloxy groups,
wherein n is an integer of from 0 to 4 and R⁷ represents a cycloalkyl group, a phenyl group or a cyclic group which is a 3- to 7-membered, aromatic or non-aromatic ring, which contains from 1 to 4 heteroatoms selected from N, O and S and which is optionally fused to an aromatic or heteroaromatic ring, the phenyl group being unsubstituted or substituted by one or more substituents selected from halogen, alkyl, aryl, heteroaryl, heterocyclyl, hydroxy, alkylenedioxy, alkoxy, alkylthio, amino, mono- or di-alkylamino, nitro, cyano, hydroxycarbonyl, alkoxycarbonyl, acylamino, carbamoyl, alkylcarbamoyl, dihydrophosphoryloxy, dialkoxyphosphoryloxy and haloalkyl groups and the cyclic group being unsubstituted or substituted by one or more substituents selected from halogen, hydroxy, alkoxy, phenyl, alkoxycarbonyl, amino, mono-alkylamino, di-alkylamino, hydroxycarbonyl, and alkyl groups, the alkyl substituents being unsubstituted or substituted by one or more further substituents selected from halogen, hydroxy, alkoxy, alkylthio, acylamino, carbamoyl, alkylcarbamoyl, dihydroxyphosphoryloxy, dialkoxyphosphoryloxy, hydroxyalkoxy, phenyl, alkoxycarbonyl, amino, mono- and di-alkylamino and hydroxycarbonyl groups;
R³ represents hydrogen, halogen, or a nitro, alkoxycarbonyl or alkyl group, the alkyl group being unsubstituted or substituted by one or more substituents selected from hydroxy, halogen, alkoxy, alkylthio, amino, mono- or di-alkylamino, hydroxycarbonyl, alkoxycarbonyl, acylamino, carbamoyl and alkylcarbamoyl groups;
R⁴ and R⁵ are the same or different and each represents hydrogen, halogen, alkyl, hydroxy, alkoxy, alkylthio, dialkylaminoalkoxy, amino, mono- or dialkylamino, nitro, cyano or haloalkyl, or R⁴ and R⁵, together with the atoms to which they are attached, form a 5 to 7 membered ring containing from 0 to 4 heteroatoms selected from N, O and S;
L₁ is a direct bond or is —O—, —S—, —N(Z)-, —O(CH₂)ₘ—, —O(CR⁸R⁹)ₘ—, —S(CR⁸R⁹)ₘ—, —CH═CH—, —(CH₂)ₘ—, —(CR⁸R⁹)ₘ, —(CH₂)ₘO—, —(CR⁸R⁹)ₘO—, —O(CH₂)ₘO—, —O(CR⁸R⁹)ₘ N(Z)- or —N(Z)(CR¹R⁹)ₘ— wherein m is an integer of from 1 to 6 and each of Z, R⁸ and R⁹ are the same or different and each represent a group selected from hydrogen, C₁-C₆ alkyl, cycloalkyl, cycloalkyl-C₁-C₆ alkyl, heterocyclyl, heterocyclyl-C₁-C₆ alkyl, aryl, aryl-C₁-C₆ alkyl, heteroaryl, heteroaryl-C₁-C₆ alkyl, hydroxy, C₁-C₆ alkoxy, halogen, cyano, C₁-C₆ alkoxycarbonyl, carbamoyl and haloalkyl, the alkyl, cycloalkyl, heterocyclyl, aryl and heteroaryl moieties being unsubstituted or substituted with one to four substituents independently selected from R¹, or Z is as defined above and R⁸ and R⁹, together with the atom to which they are attached, form a 4 to 8 membered ring; and
R⁶ represents —C(O)NR¹⁰R¹¹, —S(O)₂NR¹⁰R¹¹, —ON═CR²R³, or a heterocyclyl, aryl or heteroaryl group, the heterocyclyl, aryl and heteroaryl groups being unsubstituted or substituted with substituents R¹⁴ to R⁷, wherein:
R¹⁰ and R¹¹ are either
(a) the same or different, each independently representing hydrogen, an alkyl group, a cycloalkyl group or a phenyl group, wherein (i) the alkyl group is unsubstituted or substituted by one or more substituents selected from hydroxy, halogen, alkoxy, alkylthio, amino and mono- and di-alkylamino groups, (ii) the cycloalkyl group is optionally fused to an aromatic ring and (iii) the cycloalkyl group and the phenyl group are unsubstituted or substituted by one or more substituents selected from (1) groups of formula —(CH₂)ₙR⁷, —O—(CH₂)ₙR⁷, —S—(CH₂)ₙR⁷, —COR and —CONHR, wherein R is alkyl or —(CH₂)ₙR⁷ and n and R⁷ are as defined above, (2) groups of formula —(CH₂), —S(O)₂NR′R″ wherein n is as defined above and R′ and R″ are the same or different and are each selected from hydrogen and alkyl or form, together with the nitrogen atom to which they are attached, a 4- to 7-membered heterocyclic ring containing 1, 2 or 3 heteroatoms selected from N, O, and S, (3) groups of formula —(CH₂)ₙ—CO₂R′″ wherein n is as defined above and R′″ is hydrogen or alkyl, (4) groups of formula —N⁺ R″″₃ wherein each R″″ is the same or different and is an alkyl group, and (5) halogen atoms and alkyl, hydroxy, alkylenedioxy, alkoxy, alkylthio, amino, mono- and di-alkylamino, nitro, cyano, hydroxycarbonyl, alkoxycarbonyl, acylamino, carbamoyl, dihydroxyphosphoryloxy, dialkoxyphosphoryloxy or haloalkyl groups, the alkyl substituents being unsubstituted or substituted by one or more further substituents selected from cyano, nitro, amino, hydroxy and halogen, or
(b) are taken together with the atom to which they are attached to form, a 3- to 7-membered ring comprising up to 4 heteroatoms selected from N, O and S, which ring is (i) optionally fused to an aromatic ring or to a heteroaromatic ring which is in turn optionally fused to an aromatic ring and is (ii) unsubstituted or substituted by one or more substituents independently selected from halogen atoms, groups of formula —X—R⁷ and —CO₂—X—R⁷ wherein X is a direct bond, a C₁-C₄ alkylene group or a carbonyl group and R⁷ is as defined above, and hydroxy, cyano, nitro, oxoalkyl, carbamoyl, hydroxycarbonyl, alkoxycarbonyl, amino, mono- and di-alkylamino, divalent alkylene and alkyl groups, the alkyl substituents being unsubstituted or substituted by one or more further substituents selected from hydroxy, alkoxy, hydroxyalkoxy, amino and mono- and di-alkylamino groups, and the moiety X being unsubstituted or substituted by one or two further substituents selected from phenyl, alkyl, hydroxy and thio groups and groups of formula —CO₂R′ and —CONR′R″ wherein R′ and R″ are the same or different and are hydrogen or alkyl, or
(c) are defined so that R¹⁰ represents hydrogen or an alkyl group and R¹¹ represents a group of formula —X—R⁷ wherein X and R⁷ are as defined above;
R¹² and R¹³ are defined as R¹⁰ and R¹¹ above, except that either or both of R¹² and R¹³ can be an amino, alkylamino or dialkylamino group; and
R¹⁴ to R¹⁷ are the same or different and each independently represents hydrogen, a halogen atom, a group of formula —(CH₂)ₙ—R⁷, wherein n and R⁷ are as defined above or an alkyl group, the alkyl group being unsubstituted or substituted by one or more substituents selected from hydroxy, alkoxy, alkylthio, amino, mono- or di-alkylamino, hydroxycarbonyl, alkoxycarbonyl, acylamino, carbamoyl, alkylcarbamoyl, dihydroxyphosphoryloxy, dialkoxyphosphoryloxy and haloalkyl groups, or R¹⁴ and R¹⁵ are as defined above and R¹⁶ and R¹⁷, together with the atoms to which they are attached, form a 4 to 8 membered aromatic or non-aromatic ring which contains from 0 to 4 heteroatoms selected from N, O and S, and which is unsubstituted or substituted by one or more substituents selected from halogen atoms and alkyl, hydroxy, phenyl, alkoxycarbonyl, amino, mono-alkylamino, di-alkylamino and hydroxycarbonyl groups, the alkyl substituents being unsubstituted or substituted by one or more further substituents selected from halogen atoms and hydroxy, alkoxy, alkylthio, acylamino, carbamoyl, alkylcarbamoyl, dihydroxyphosphoryloxy, dialkoxyphosphoryloxy, hydroxyalkoxy, phenyl, alkoxycarbonyl, amino, mono- or di-alkylamino and hydroxycarbonyl groups.

2. A compound according to claim 1, wherein R¹ and R² are the same or different and each independently represent hydrogen, a C₁-C₄ alkyl group which is unsubstituted or substituted by 1 or 2 substituents selected from C₁-C₄ alkoxy and C₁-C₄ alkylthio substituents, a group of formula —(CH₂)ₙ—(C₃-C₆ cycloalkyl) or a group of formula —(CH₂)ₙ-(morpholino) wherein n is as defined above in claim 1.

3. A compound according to claim 1, wherein R³ represents hydrogen, halogen or C₁-C₄ haloalkyl.

4. A compound according to claim 1, wherein R⁴ and R⁵ are the same or different and each represent hydrogen, C₁-C₆ alkyl, hydroxy, C₁-C₆ alkoxy, C₁-C₆ alkylthio, amino or C₁-C₆ alkylamino.

5. A compound according to claim 1, wherein Z, R⁸ and R⁹ are hydrogen, C₁-C₆ alkyl, or phenyl.

6. A compound according to claim 1, wherein L₁ is —O(CH₂)ₘ—, —O(CR⁸R⁹)ₘ—, CH═CH, (CH₂)ₘ—, —(CR⁸R⁹)ₘ—, —(CH₂)ₘO—, —(CR⁸R⁹)ₘO—, —O(CH₂)ₘO— or —(CR⁸R⁹)ₘ N(Z)-, wherein m is from 1 to 4 and R⁸, R⁹ and Z are as defined in claim 1.

7. A compound according to claim 1, wherein R¹² and R¹³ are the same or different and each represent amino, mono- or di-(C₁-C₄ alkyl)amino or phenyl, the phenyl group being unsubstituted or substituted by one or two substituents selected from halogen, C₁-C₄ alkoxy, C₁-C₄ alkyl, hydroxy, amino mono-(C₁-C₄ alkyl)amino and C₁-C₄ haloalkyl.

8. A compound according to claim 1, wherein R⁷ is:
a C₃-C₆ cycloalkyl group; or
a phenyl group which is unsubstituted or substituted with 1, 2 or 3 substituents selected from halogen, C₁-C₄ alkyl, aryl, heteroaryl, hydroxy, C₁-C₄ alkylenedioxy, C₁-C₄ alkoxy, C₁-C₄ alkylthio, amino, mono- and di-(C₁-C₄ alkyl)amino, nitro, cyano, hydroxycarbonyl, (C₁-C₄ alkoxy)carbonyl, (C₂-C₇ acyl)amino, carbamoyl, (C₁-C₄ alkyl)carbamoyl, dihydrophosphoryloxy, di-(C₁-C₄ alkoxy)phosphoryloxy and C₁-C₄ haloalkyl groups; or
a cyclic group which is a 3- to 7-membered aromatic or non-aromatic ring containing from 1 to 4 heteroatoms selected from N, O and S and which is optionally fused to an aromatic ring, which group is unsubstituted or substituted by 1, 2 or 3 substituents selected from halogen, hydroxy, C₁-C₄ alkoxy, phenyl, C₁-C₄ alkoxycarbonyl, amino, mono-(C₁-C₄ alkyl)amino, di-(C₁-C₄ alkyl)amino, hydroxycarbonyl and C₁-C₄ alkyl groups, the alkyl substituents being unsubstituted or substituted by 1 or 2 further substituents selected from halogen, hydroxy, C₁-C₄ alkoxy, C₁-C₄ alkylthio, C₂-C₇ acylamino, carbamoyl, C₁-C₄ alkylcarbamoyl, dihydroxyphosphoryloxy, di-(C I—C₄ alkoxy)phosphoryloxy, hydroxy-(C₁-C₄ alkoxy)-, phenyl, C₁-C₄ alkoxycarbonyl, amino, mono- and di-(C₁-C₄ alkyl)amino and hydroxycarbonyl groups.

9. A compound according to claim 8, wherein the cyclic group is a 5- or 6-membered aromatic or non-aromatic ring containing 1 or 2 heteroatoms selected from N, O and S.

10. A compound according to claim 9, wherein the substituents on the cyclic group are selected from halogen, hydroxy, phenyl, C₁-C₄ alkoxy, amino, mono- and di-(C₁-C₄ alkyl)amino, C₁-C₄ alkyl, C₁-C₄ haloalkyl, hydroxy-(C₁-C₄ alkyl)- and phenyl-(C₁-C₄ alkyl)-.

11. A compound according to claim 8, wherein, R⁷ is a phenyl group, which is unsubstituted or substituted by 1 or 2 substituents selected from halogen, C₁-C₄ alkyl, phenyl, hydroxy, C₁-C₄ alkoxy, C₁-C₄ alkylthio, amino, mono- and di-(C₁-C₄ alkyl)amino and C₁-C₄ haloalkyl groups.

12. A compound according to claim 1, wherein, when the moiety X is substituted, R⁷ is a phenyl group as defined in claim 1.

13. A compound according to claim 1, wherein, when R¹⁰ and R¹¹ are defined according to option (a), they are the same or different and each represent hydrogen, a C₁-C₆ alkyl group, a phenyl group or a C₅-C₆ cycloalkyl group optionally fused to a phenyl ring, the alkyl group being unsubstituted or substituted by 1 or 2 substituents selected from hydroxy, halogen and amino groups and the phenyl and cycloalkyl groups being unsubstituted or substituted by 1, 2 or 3 substituents selected from (1) groups of formula —(CH₂)ₙR⁷, —O—(CH₂)ₙ—R⁷, —COR and —CONHR wherein R is C₁-C₄ alkyl or —(CH₂)ₙR⁷, n is 0, 1 or 2 and R⁷ is as defined in claim 1, (2) groups of formula —(CH₂)ₙ—S(O)₂—NR′R″, wherein n is 0 or 1 and R′ and R″ are the same or different and are hydrogen or C₁-C₄ alkyl or, together with the N atom to which they are attached, form a pyrrolidinyl or piperidyl ring, (3) groups of formula —(CH₂)ₙ—CO₂R′″ wherein n is 1 or 2 and R′″ is hydrogen or C₁-C₄ alkyl, (4) groups of formula —N⁺R″″₃ wherein each R″″ is the same or different and is a C₁-C₄ alkyl group, and (5) halogen atoms and C₁-C₄ alkyl, hydroxy, C₁-C₄ alkoxy, amino, mono- and di-(C₁-C₄ alkyl)amino, nitro, cyano, hydroxycarbonyl, C₁-C₄ alkoxycarbonyl, (C₃ to C₅ acyl)amino, carbamoyl and C₁-C₄ haloalkyl groups, the alkyl substituents being unsubstituted or substituted by a further substituent selected from cyano, nitro, amino, hydroxy and halogen.

14. A compound according to claim 1, wherein when R¹⁰ and R¹¹ are defined according to option (b), they form, together with the nitrogen atom to which they are attached to form, an aromatic or non-aromatic 5- or 6-membered ring containing 1 or 2 heteroatoms selected from N, O and S, which ring is optionally fused to a phenyl ring or to an indole group, and is unsubstituted or substituted by 1, 2 or 3 substituents independently selected from halogen atoms, groups of formula —X—R⁷ and —CO₂—X—R⁷ wherein X and R⁷ are as defined in claim 1 and hydroxy, cyano, nitro, C₁-C₄ alkoxycarbonyl, amino, C₁-C₂ divalent alkylene and C₁-C₄ alkyl groups.

15. A compound according to claim 1, wherein when R¹⁰ and R¹¹ are as defined in option (c), R¹⁰ is hydrogen or a C₁-C₄ alkyl group and R¹¹ is a group of formula —X—R⁷ wherein:
X is a direct bond, a C₁-C₄ alkylene group or a carbonyl group, wherein the C₁-C₄ alkylene group is unsubstituted or substituted by 1 or 2 substituents selected from C₁-C₄ alkyl, hydroxy, —CO₂H and —CO₂—(C₁-C₄ alkyl) groups; and
R⁷ is a cyclopentyl, cyclohexyl, benzimidazolyl, benzothiazolyl, thiadiazolyl, thienyl, pyrimidinyl, pyrazinyl, isoxaolyl, pyrazolyl, furanyl, pyridyl, pyrimidinyl, phenyl or piperidinyl group, the pyridyl, pyrimidinyl, piperidinyl, thiadiazolyl and furanyl groups being unsubstituted or substituted by 1 or 2 substituents selected from halogen atoms and hydroxy, C₁-C₄ alkoxy, phenyl-(C₁-C₄ alkyl)- and C₁-C₄ alkyl groups and the phenyl, benzothiazolyl and benzimidazolyl groups being unsubstituted or substituted by 1 or 2 substituents selected from halogen atoms and hydroxy, C₁-C₄ alkoxy and C₁-C₄ alkyl groups,
provided that when X is substituted, R₇ is an unsubstituted phenyl group.

16. A compound according to claim 1, wherein R¹⁴ to R¹⁷ are the same or different and each independently represent hydrogen, a halogen atom, a 5- or 6-membered heteroaryl group having 1 or 2 heteroatoms selected from N, O and S, a C₁-C₄ alkyl group or a phenyl group which is unsubstituted or substituted by 1 or 2 substituents selected from halogen atoms, C₁-C₄ alkyl groups and C₁-C₄ haloalkyl groups, or R¹⁴ and R¹⁵ are as defined above and R¹⁶ and R¹⁷, together with the atoms to which they are attached, form a 5- or 6-membered aromatic or non-aromatic ring which contains 0, 1 or 2 heteroatoms selected from N, O and S and which is unsubstituted or substituted by 1 or 2 substituents selected from C₁-C₄ alkyl, phenyl and phenyl-(C₁-C₄ alkyl)-groups.

17. A compound according to claim 1, wherein R⁶ represents —C(O)NR¹⁰R¹¹, wherein R¹⁰ and R¹¹ are as defined in claim 1, —ON═CR¹²R¹³, wherein R¹² and R¹³ are as defined in claim 1, a phenyl group or a 5- or 6-membered heteroaryl or heterocyclyl group, which group contains 1, 2 or 3 heteroatoms selected from N, O and S, wherein the phenyl, heteroaryl or heterocyclyl group is unsubstituted or substituted with substituents R¹⁴ to R¹⁷, as defined in claim 1.

18. A compound according to claim 17, wherein the heteroaryl or heterocyclyl group is a 6-membered heteroaryl group having 1 or 2 heteroatoms selected from N, O and S, or a group of formula (H) 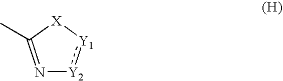
wherein X represents O, S or N, and the 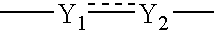
moiety represents —N═C(R¹⁸)—, —C(R¹⁸)═N—, —C(R¹⁸)═C(R¹⁹)—, or —CH(R¹⁸)—CH(R¹⁹)—, wherein R¹⁸ and R¹⁹ are the same or different and each represent hydrogen, a group of formula —(CH₂)ₙ—R⁷, wherein n and R⁷ are as defined in claim 1, or an alkyl group, the alkyl group being unsubstituted or substituted by one or more substituents selected from hydroxy, alkoxy, alkylthio, amino, mono- and di-alkylamino, hydroxycarbonyl, alkoxycarbonyl, acylamino, carbamoyl, alkylcarbamoyl, dihydroxyphosphoryloxy, dialkoxyphosphoryloxy and haloalkyl groups, or R¹⁸ and R¹⁹, together with the atoms to which they are attached, form a 4- to 8-membered aromatic or non-aromatic ring, which contains from 0 to 4 heteroatoms selected from N, O and S and which is unsubstituted or substituted by one or more substituents selected from halogen atoms and alkyl, hydroxy, phenyl, alkoxycarbonyl, amino, mono-alkylamino, di-alkylamino and hydroxycarbonyl groups, the alkyl substituents being unsubstituted or substituted by one or more further substituents selected from halogen atoms and hydroxy, alkoxy, alkylthio, acylamino, carbamoyl, alkylcarbamoyl, dihydroxyphosphoryloxy, dialkoxyphosphoryloxy, hydroxyalkoxy, phenyl, alkoxycarbonyl, amino, mono- and di-alkylamino and hydroxycarbonyl groups.

19. A compound according to claim 18, wherein R¹⁸ and R¹⁹ are the same or different and each independently represent hydrogen, a 5- or 6-membered heteroaryl group having 1 or 2 heteroatoms selected from N, O and S, a C₁-C₄ alkyl group or a phenyl group which is unsubstituted or substituted by 1 or 2 substituents selected from halogen atoms, C₁-C₄ alkyl groups and C₁-C₄ haloalkyl groups, or R¹⁸ and R¹⁹, together with the atoms to which they are attached, form a 5- or 6-membered aromatic or non-aromatic ring which contains 0, 1 or 2 heteroatoms selected from N, O and S and which is unsubstituted or substituted by 1 or 2 substituents selected from C₁-C₄ alkyl, phenyl and phenyl-(C₁-C₄ alkyl)-substituents.

20. A compound according to claim 17, wherein R⁶ represents —C(O)NR¹⁰R¹¹, wherein R¹⁰ and R¹¹ are as defined in claim 1, —ON═CR¹²R¹³ wherein R¹² and R¹³ are as defined in claim 1, a phenyl group optionally substituted by a halogen atom or a 5- or 6-membered heteroaryl or heterocyclyl group which is optionally fused to a phenyl ring and which is unsubstituted or substituted by 1 or 2 substituents selected from phenyl, pyridyl, phenyl-(C₁-C₄ alkyl)-, C₁-C₄ alkyl and piperidylidene substituents, the phenyl substituents being unsubstituted or substituted by 1 or 2 further substituents selected from halogen atoms and C₁-C₄ alkyl groups and the piperidylidene substituents being unsubstituted or substituted by 1 or 2 further substituents selected from phenyl, phenyl-(C₁-C₄ alkyl)- and C₁-C₄ alkyl groups.

21. (canceled)

22. A pharmaceutical composition comprising a compound according to claim 1 and a pharmaceutically acceptable carrier or diluent.

23. A method of reducing or preventing mast cell degranulation in a subject in need of such treatment, which method comprises administering to the said subject an effective amount of a compound according to claim 1.

24. A method according to claim 23, wherein the subject is suffering from or susceptible to a disorder which is asthma, bronchoconstriction, allergic potentiation, inflammation or reperfusion injury, myocardial ischemia, inflammation, a diarrheal disease, brain arteriole diameter constriction, Parkinson's disease, non insulin dependent diabetes mellitus, release of allergic mediators or an autoimmune disease.

25. A method according to claim 24, wherein the autoimmune disease is Addison's disease, autoimmune hemolytic anemia, Crohn's disease, Goodpasture's syndrome, Grave's disease, Hashimoto's thyroiditis, idiopathic thrombocytopinic purpura, insulin-dependent diabetes mellitus, multiple sclerosis, myasthenia gravis, pemphigus vulgaris, pernicious anemia, poststreptococcal glomerulonephritis, psoriasis, rheumatoid arthritis, scleroderma, Sjogren's syndrome, spontaneous infertility, and systemic lupus erythematosus.

26. A method according to claim 25, wherein the said allergic potentiation is an allergic reaction.

27. A method according to claim 26, wherein the allergic reaction is rhinitis, a poison ivy induced allergic response or urticaria.

28. A method according to claim 24, wherein the reperfusion injury is myocardial reperfusion injury and/or the inflammation is inflammatory bowel disease.

29 and 30. (canceled)
